(19) United States
(12) Patent Application Publication
(10) Pub. No.: US 2007/0270658 A1
(43) Pub. Date: Nov. 22, 2007

(54) Quantum memory fusion

(76) Inventor: Frederick Mitchell, Riverside, CA (US)

Correspondence Address: DR. FREDERICK MITCHELL, P.O. BOX 392, HEMET, CA, 92546-0392, US

(21) Appl. No.: 11/219,623

(22) Filed: Sep. 3, 2005

Related U.S. Application Data
(60) Provisional application No. 60/607,306, filed on Sep. 3, 2004.

(51) Int. Cl. A61B 5/00

(52) U.S. Cl. 600/300

(57) ABSTRACT

A non-invasive method for storing, editing, deleting, appending, and retrieving infinite quantities of audio, visual or tactile data from targeted neuronal groups in the human brain. The process involves transmitting sequenced, buffered, and indexed data streams to human sensory receptors. Each data stream contains an ordinal or cardinal position that identifies a targeted group of neurons. Linear data streams may be stacked vertically and then transmitted to manufacture complete thoughts. Both linear and stacked data streams create artificial memory constructs. The manufactured memories appear older than they are because of the indexed and undated artificial mnemonic environmental variables embedded in the data stream. This method further creates a genius complex in some individuals whereby implanted memories are recalled quickly and precisely using a sequenced query that specifies the neuronal group. In stark contrast, the brain indexes naturally occurring memories with non-indexed and unknown variables randomly selected by the human senses.

### CROSS REFERENCE TO RELATED APPLICATION

This application is in reference to U.S. provisional patent application Ser. No. 60/607,306 entitled “Quantum Memory Fusion” filed on Sep. 3, 2004.

### BACKGROUND OF THE INVENTION

1. Field of Invention

This invention relates to mnemonic, learning aid, behavior modification, and tactile and abstract audio and visual methods using quantum memory fusion, the only known method that targets the neurons in the brain, records, erases or updates memories and produces as a result tangible improvements in a user's cognitive abilities or knowledge or behavior in sixty-minutes or less, and is applicable to all forms of known media and can be transmitted in all known languages, and used by all cognizant persons who possess functioning ocular or auricular senses or other sensory receptors. The present invention will be used in medical, psychiatric, psychotherapeutic, hypnotherapeutic, educational, self-help, personal development, home, entertainment, film, cinema, drama and stage, government, political, military, law enforcement, rehabilitative, sports, manufacturing, commercial and industrial, advertising, toy, game, music, language and linguistics, religious, zoological, veterinary, and clinical research environments and mass produced quickly and inexpensively with computer hardware and computer software.

2. General Discussion of the Prior Art

Existing products commonly found in the market place include mnemonic, learning aid, behavior modification, and tactile and abstract audio and visual devices and methods, presented in and on a plurality of formats, encompassing a plurality of subjects, and transmitted to an individual via one or more sensory receptors. It is well established that mnemonic methods and devices purportedly improve memory, that learning aid methods and devices purportedly improve subject matter retention, that behavior modification methods and devices purportedly improve physiological cognitive activity, and that tactile and abstract audio and visual methods and devices purportedly influence incognizant behavior. Consequently, the use of the aforementioned methods and devices is known in the prior art. More specifically, methods hereto devised and utilized for the aforementioned purposes are known to consist of basically familiar, expected and obvious configurations and methods, and limited results, notwithstanding the myriad of methods and configurations encompassed by the crowded prior art which have been developed for the fulfillment of countless objectives and requirements, the collective theories and presuppositions of which are incorporated herein and by reference.

The first main problem is that conventional scientific theory attempts to explain mnemonics as a mysterious abstract of the human process. Frequently, theorists use ambiguous terms such as “long-term memory” or “thought” or “emotion” to support the mystery hypothesis. Consequently, methods, devices, and apparatus depicted by the prior art are therefore incorrectly mnemonically applied. Hence, the limited or imperceptible results achieved, as my invention will later prove.

The second main problem is that it is unspecified and unknown in the prior art the role that mnemonics serve in abstract ocular and auricular applications transmitted at the sub-threshold. Failure to understand and abide by this covenant forces neuronal memory blocks to relegate the abstract content of the prior art to mere fantasy or phantom construct. Because popular scientific theory and otherwise believed concepts, previous ideas, products, devices, books, papers, apparatus and all of the prior art ignored this significant relationship, abstract audio content is therefore erroneously produced and administered to users of the prior art.

Another problem is that the prior art references and cites, but can not answer why the experiment performed by Becker U.S. Pat. No. 3,278,676 (1966) was not completely successful or why it produced any result at all.

In addition to the above problems within the prior art, several additional problems are described in the following Factors 2.a to 2.y.
2.a. The prior art can not produce tangible results in sixty-minutes (one hour) or less.
2.b. The prior art can not create an intra-cranial harmonic “voice” that literally speaks from within the molecular structure of neurons in the brains of users, and that reinforces the data and assists users in problem solving.
2.c. The results yielded, if any, from the prior art are speculative. Neither objective viewers nor other scientist can measure, quantify, repeat, or record the results with any accuracy.
2.d. Second and subsequent users of the prior art, to include a global population of users, can not duplicate the results that a first user may obtain from the prior art.
2.e. Even the limited results yielded by the prior art are not permanent. After more than twenty-five years of consistent, serious investigation and experimentation, scientists and notable celebrities practicing in the sub-threshold, mnemonic, and behavior modification industries are incapable of reaching every living person with inexpensive products that produce tangible, measurable, immediate, and permanent results.
2.f. Users of the prior art can not remember or recite from, nor directly or immediately benefit from treatments offered by the prior art.
2.g. The prior art can not exploit the unfiltered storage capabilities of the right cerebral hemisphere.
2.h. The prior art fails to capitalize on the raw processing power of the right cerebral hemisphere.
2.i. The prior art fails to capitalize on the high-speed data absorption capabilities of the right cerebral hemisphere.
2.j. The prior art fails to benefit from the virtually unlimited storage capabilities of the right cerebral hemisphere.
2.k. The prior art fails to capitalize on the creative problem solving ability of the right cerebral hemisphere.
2.l. The prior art requires long-term use to effect even modest behavior modification, but the general consensus is that said modifications are so slight as to be intangible or that the results conceivably represent the byproduct of an extraneous, unexplained external stimulus such as time or contact with other individuals or groups.
2.m. The prior art fails to originate tactile abstract synchronization for cognitive physiological response.
2.n. The prior art relies heavily on sub-threshold abstracts that appear as fantasy and phantom constructs at the molecular mnemonic level.
2.o. The prior art induces mnemonic indifference and rejection to unsubstantiated sub-threshold abstract constructs.
2.p. The prior art requires a user to concentrate on the product instead of the treatment, such as learning software functions or responding to synthetic tactile queues.
2.q. Apparatuses promoted within the prior art are typically hard to find, difficult to construct, immobile, bulky, expensive, require training before potential benefit, require ancillary programs to function, or need assistance and supervision from trained persons for role play or role modeling.
2.r. The prior art induces discomfort from wearing goggles or lenses; electrodes attached to a user's body, wearing external sensory input or output devices, or encapsulation in a closed room or other containment structure.
2.s. The prior art can not fuse data directly to any portion of a brain.
2.t. The prior art can not retrieve data fused to a brain.
2.u. The prior art can not erase data previously fused to a brain.
2.v. The prior art can not update or modify data previously fused to a brain.
2.w. The prior art can not create specific directory structures on the brain and later fuse data into said directories.
2.x. The prior art contains no formula that is easy to understand by those unskilled in the art.
2.y. The prior art can not be utilized with every form of known media, to include, written text, the spoken word, still imagery, motion pictures, and music.

Another problem is that the widely accepted definition of “sub” when combined with “limen” is inaccurate and misleading because of the prior art.

Then another problem is that the use of the phrase “long-term memory” is incomplete as used in neuroscience.

Other problems within the prior art will become apparent in later sections.

3. Prior Art—Audio Visual Behavior Modification

Barwick, in U.S. Pat. No. 4,459,114 (1984) suggests a system that allows a trainee to first view and later interact during role-play with pre-recorded information that represents a skill or behavior desired by the trainee. Barwick's system is obvious because people often rehearse in front of a mirror, video tape a presentation, or create an audio recording of a speech or lecture for self critique. These home-methods are inexpensive and private. Barwick fails to indicate the plurality of trainers required in producing the training videos if in fact role modeling is the objective and not role-playing. Trainees that demonstrate matching traits of their trainers will more effectively emulate the pantomimes of trainers whom they consider role models. The plurality of role models required create an obvious expense. The difficulty of determining whom the trainee perceives as a role model practically renders this device unusable in commercial settings.

Dennis, in U.S. Pat. No. 4,734,038 (1988) offers mnemonic devices as cues to create a desirable behavior as demonstrated by a video role model. By associating a positive behavior with an inanimate object and then a role model, Dennis creates a situation wherein all elements of the modification must be present to “trigger” the new behavior. This method fails because at least one element of the modification may be inaccessible during daily activities.

Frenkel and Frenkel, in U.S. Pat. No. 4,327,712 (1982) describes a system that encompasses a mirror and lights and that reflects one's own facial image. There is no empirical evidence to support the connotation that what one sees in the mirror (as a reflection) is a catalyst for behavior modification. Seeing ones facial expression is of no benefit in decision making because decision making begins as a neurological process, not as a physical process, regardless of the tangibles of the thing to be decided. The associated facial expression is the result of the emotions attached to the process of contemplation based on primary emotionality. They are not the result of the manifested decision. The Frenkel process is counterproductive because it interrupts the cognitive motor function signals from the right cerebral hemisphere, which is responsible for primary emotionality, which manipulates facial muscle contractions based on neuronal signaling of a spatial expression, creating physiological vacillation that falsely attests to ones state of being. Thus, the therapeutic value of the Frenkel device in psychiatry is seriously questionable as the device is in direct opposition of healthy neurological responsiveness. Repression of emotions or feelings may cause personality disorders, antisocial behavior, depressive disorders, substance abuse, or suicide.

Densky, in U.S. Pat. No. 4,717,343 (1988) devised an associative behavioral model that suggests that images of a perceived undesired habit be shown to a subject person first followed by opposing images of a desired habit. The perceived undesired habit is reinforced due to repeated viewing of images representing the undesired habit. The right parietal temporal cortex stores both sets of images without rationalization, but with emotionality. Based on Densky's patent, the two images conflict in actual and symbolic representation because the perceived undesired habit has substantial mnemonic reinforcement. That is, it is already a condition in permanent recallable memory on which other conditions will rely. The image with the greatest reinforcement will survive the subconscious conflict, as is the case with cigarette smoking, alcohol abuse, drug abuse, and other chronic dependencies. Circumventing the euphoriant effects of chronic dependency requires substantial mnemonic reinforcement. Similar prior art is exemplified in U.S. Pat. No. 5,017,143 to Backus et al. (1991)

Weathers, in U.S. Pat. No. 5,219,322 (1993) outlines an apparatus that elicits negative emotions or behaviors from a patient via ocular and auricular stimuli. An attending therapist directs the patient to modify his or her subconscious associations to the negative experiences, in a method known as Neuro Linguistics Programming. This prior art reference is similar to the Densky reference insofar as the methods used to modify behavior, object of the methods, and the potential value of the results. Thus, this method also fails to achieve permanent behavior modification.

D'Alitalia and Mead, in U.S. Pat. No. 5,784,124 (1998) use supra-abstract messages superimposed over a video display to achieve a theoretical abstract effect by displaying said messages for brief periods. This method is obvious and the results speculative. In the Moore experiment 2004, it was proven the supra-abstract messages produce limited results if any. Further, the cost of the D'Alitalia and Mead product versus an insignificant probability of success disqualifies this prior art as a viable means for behavior modification.

Borg, in U.S. Pat. No. 5,833,466 (1998) illustrates a portable device that a user activates when the user experiences an urge to perform or participate in an undesirable acquired behavior. The user selects from a plurality of symbols representing alternate responses to the acquired behavioral response. The user performs one or more of the alternate responses. Borg's alternate response method pose a myriad of disadvantages for its user. The list of alternate responses may be unsuitable to the user, creating a hesitancy for compliance. Borg relies on the user for cognitive obedience to the alternate response. Borg's device relies on the user to determine if an acquired behavioral response is an undesired behavior. Borg's device relies solely on the user to interrupt the acquired behavior using raw will-power. Will-power is an unknown variable, it is not a constant on which psychologists, or psychiatrists can rely. Will-power is an illusory means of behavior modification.

Kanter, in U.S. Pat. No. 6,497,577 B2 (2002) offers a device that helps a user identify an emotional problem, assigns the problem a spiritual correlate, and provides a secular antidote to the problem. Kanter offers no desirable habit-forming actions based on the single secular solution. After identifying the emotional problem, a user continues to identify the same problem repeatedly without the benefit of guided activities varying in scope and intensity. Because Kanter offers both a religious and practical solution, users may become confused with this theological secularist approach.

Chai, in U.S. Pat. No. 6,615,197 B1 (2003) outlines a method involving electrical impulses associated with audible and visual stimuli to increase human information processing capacity. First, it is impossible to increase the capacity of the human brain because humans can not synthetically manufacture neurons. Second, it is impossible to improve the information processing capacity of the human brain because the brain processes all information at the same rate, as my invention will later prove. Next, Chai use vibratory devices, and other devices attached to the user, carried on the user's person, or placed in the user's environment. Because the stimulus is known, repeated, and anticipated according to Chai, a similar outcome could be achieved using common flash cards or by reading the information again, thus reducing the latency of the response due to the new mnemonic condition, thereby decreasing the subsequent error rate and response time (the process of learning). Ivan Petrovich Pavlov (1849-1936) established in 1905 that any external agent could, by coinciding in time with an ordinary reflex, become the conditioned signal for the formation of a new conditioned reflex. Here, the new conditioned reflex is remembering versus forgetting. Chai's method is useful for experimental purposes, but at the same time exorbitantly wasteful for any commercial application. Other behavior modification and behavior enhancement prior art is illustrated in U.S. Pat. Nos. 4,127,943 to Mitchell et al. (1978), 4,373,918 to Berman (1983), 4,699,153 to Shevrin et al. (1987), 4,902,274 to Gleeson, III (1990), 5,149,317 to Robinson (1992), 5,312,114 to Lipson (1994), 5,450,855 to Rosenfeld (1995), 5,823,932 to Speigel (1998), 5,899,867 to Collura (1999), 6,129,748 to Kamei (2000), 6,595,779 to Rose (2003), 6,648,649 B2 to Rappaport (2003), and 6,650,251 B2 to Gerrity (2003), the teachings of each incorporated herein by reference.

4. Prior Art—Abstract Sub-Threshold Methods and Apparatuses

U.S. Pat. No. 3,060,795 to Corrigan et al. (1962) and U.S. Pat. No. 3,278,676 to Becker (1966) exemplifies some early research into the effects of abstract audio and video for sub-threshold transmissions. Lundy, in U.S. Pat. No. 4,395,600 (1983) furthers the prior art of Becker and devised an auditory sub-threshold message system and method whereby the abstract messages are constantly masked by a circuit that adjusts the amplitude of the abstract messages depending on the ambient noise in the environment, so as to audibly conceal the message. Lundy designed the prior art for the sole purpose of delivering abstract sub-threshold messages to the customer shopping area of stores in an effort to deter shoplifting. The effective use of this product requires a plurality of audio sensors in a plurality of locations within the shopping. Ambient sounds in one area of a store may fluctuate differently than in other areas of the store. Depending on the size of the shopping area, the cost of the sensors and the probability of tactile output within the shopping area could be a serious deterrent to using the prior art.

Mould, in U.S. Pat. No. 4,692,118 (1987) introduced a device consisting of two panels that attach to opposite sides of a television's housing. The device claims to deliver abstract messages to a viewer while the viewer focuses on the television screen. The device is bulky, protrudes, and attaches to an expensive appliance. The more the viewer attempts to focus on the screen, the harder it becomes to ignore the large panels protruding from the sides of the housing, thereby changing the abstract nature of the prior art to distracting tactilely visual messages. Mould proposes to display negatively phrased behavior modification messages in hopes of soliciting positive behavior. Once the device distracts the viewer, each message becomes tactile. Learned pessimism of the left cerebral hemisphere will filter each message as it compares each message to the relaxing euphoric feelings of the old habits that Mould visually reinforces. As in Densky's patent, reinforcing a perceived negative behavior is of no intrinsic value to the user. Mould proposes to use a color such as red to highlight the negative phrases. Should Mould use the favorite color of the viewer, then the messages lose their impact in a visual conflict. Moreover, negative inferences have weakened for certain colors because of the logos of large corporations. For example, the colors red and yellow are the color schema of the world's largest and most successful fast food franchiser. Mould suggests using motorized scrolls of paper to display messages on each panel. Mould would need at least two separate motors and two separate scrolls of paper. Mould's device becomes noisy, distracting, environmentally unfriendly, energy hungry, and cumbersome.

Schultz, in U.S. Pat. No. 4,777,529 (1988) sets out an auditory abstract media programming system and method of encoding, adding security tones, and then decoding abstract messages. According to Schultz, a user must purchase a decoder and abstract media from a manufacturer. The user is bound to the manufacturer for continued purchases because the security tones are unique to the manufacturer. The user has no control over the content of the abstract messages. The need for users to purchase decoders, the inability of users to customize the messages, the expense of purchasing multiple decoders from a plurality of manufactures of abstract media, and the requirement that users must continue purchasing media from the same manufacture to avoid owning numerous unique decoders make the prior art undesirable for mass commercialization, simple self improvement, behavior modification, and learning.

Taylor et al., in U.S. Pat. No. 5,170,381 (1992) introduces a method for mixing abstract recordings. Taylor uses an expansive system of audiocassette recorders to create abstract messages. Taylor records each abstract message in a first voice of a man, a second voice of a woman, and a third voice of a child. On a first channel, the abstract message transmits backwards. On a second channel, the abstract message transmits forwards. Music transmits on a third primary channel. Now, in the first ear, the transmission is inbound, but in the second ear, the transmission is outbound. The three channels transmit in what Taylor describes as a “round-robin” method, wherein the transmissions appear to encircle the head of a user (much like a halo). On the first channel, Taylor records a message as a directive such as “I am successful” which transmits to the right brain of a user. On the second channel, Taylor records a similar message permissively such as “It is ok to be successful” that transmits to the left brain of a user. The two abstract channels transmit as an echo reverberation, with all three channels again encircling the head of the user, in what Taylor defines as a “whole-brain” approach. Numerous drawbacks exist in Taylor's methods. First, if a user is deaf in one or both ears, Taylor's methods fail. Second, the user invests substantially in single purpose equipment, the elaborate arrays of audiocassette decks are expensive and not readily available, the user needs an advanced knowledge of electronic circuitry, and the user must solicit two other people to record a simple self-help message. Taylor's methods are impractical for the average person. Third, a child can not record messages relating to sexual dysfunction, intimacy, and other mature, but every day subjects, thereby severely limiting product use in the adult audience. Fourth, the right hemisphere of the brain can not “act” on a message. The “whole brain” must participate in message retrieval in unison. The message must exist in the permanent recallable memory in the right hemisphere, which means it must be cognizant now or it must have been cognizant at some time in the past. Short-term memory exists in the left hemisphere. Short-term memory is brief, transient, and stores information for mere seconds. Thus, an abstract message delivered to the right auricular orifice exclusively can not produce a permanent recallable memory in the left hemisphere of the brain. The permissive statements delivered to the left brain must be consciously audible; else, they are fantasy phantom constructs that enter and exit short-term memory with an approximate duration equivalent to the actual transmission. Consequently, the channel of abstract messages transmitted to the left hemisphere of the brain create no memories. Therefore, the messages will never produce a conscious response and serve no purpose for the user. Again, Taylor's methods fail. Fifth and yet another problem is that Taylor's “round-robin” claim contradicts known neuropsychological functions. A persuasive statement delivered at the right emotional state or repetitive statements delivered during the developmental stage or over time will have an effect on an individual's behavior, as is the case in the Becker experiment exemplified in U.S. Pat. No. 3,278,676 (1966), Stockholm syndrome, and Helsinki syndrome. By way of demonstrations, a captive held at gunpoint is told the he or she will die if he or she does not acquiesce to the wishes of the captor. Because the captive's recourse is primordial states of fear and self-preservation, which are known functions of the right hemisphere of the brain, the captive hears the statement “you will die” tactilely because adrenaline production is a response to cognizant stimuli such as fear. The ensuing behavior of the captive is prima facia evidence of neuronal registration of the audible but perceived abstract message. However, had the captor not spoken to the captive, the interpretation could be substantially different. A perceived abstract message of simple theft would also be rational thinking. Nevertheless, the message is still cognizant because the captive can see the gun-wielding captor. Hence, every physiological reaction by the captive is the direct result of the cognizant stimulation of the captor. Therefore, abstract messages alone will produce no tangible result. In another example, a child is repeatedly told over years that “you will never amount to anything.” The child would begin to believe this statement in the absence of at least one memory that contradicts the statement. If no memory exists, the statement accumulates reinforcement as it engages the brain over time because neuronal memory blocks seek to validate their existence by confirming sensory input. This accumulation of neuronal memory block attachments and reinforcement will create the single recallable instance of the statement. Subsequent emotions and behaviors are inarguable results of neuronal registration of a cognizant statement delivered over time. Sixth, another problem in Taylor's method centers on his use of multiple authors on the same abstract channel, producing a variety of forward and backward, spoken and sung recordings. Humans think spatially, void of laryngeal percussion, but with bioelectrical impulses capable of generating a “harmonic” voice, as my invention will later prove. At the molecular level, the authors of a recording are irrelevant. Although humans will recognize a voice, memories of spoken words are voiceless when retrieved. Cognizant persuasion and repetition are relevant. Inasmuch, neither the author, the ear, nor the direction in which an abstract message transmits is relevant. Related prior art provides irrefutable evidence that a person engaged in thought will generate certain brain wave frequencies as exemplified in U.S. Pat. No. 3,884,218 to Monroe (1978) and U.S. Pat. No. 6,615,197 B1 to Chai (2003). Ergo, thoughts are the results of memories; memories are the results of cognizant stimuli. Seventh, Taylor cites the work of Becker in U.S. Pat. No. 3,278,676 (1966). In 1979 Becker broadcast two abstract messages “Thou shall not steal” and “Honesty is the best policy” throughout a New Orleans supermarket. Becker masked the messages in music. Becker did not alert the customers or the supermarket staff to the existence of the messages. Becker's messages are purportedly responsible for a substantial reduction in cash shortages, inventory damage, and pilfering. Becker encouraged the belief that an abstract message can cause innocuous behavior modification. Taylor's recitation of Becker's experiment is misleading. Taylor states that abstract messages seem to cause behavior to override reason. Taylor, like the scientific community at large, is easily refuted. An abstract message alone can not override reason or modify behavior. An abstract message may signal existing reasoning stored in the permanent recallable memory of the individual. Eighth, Taylor states that echo reverberation helps to ensure that the message signal is not lost and thereafter claims that echo reverberation benefits the user. Echo reverberation changes only the amplitude modulation and wavelength of sound. Echo reverberation is mutually exclusive of signal gain or loss because the signal is actually duplicated or quadrupled, with a first signal repeated slightly out of phase from a second, third and fourth signal if present. The amplitude of each signal decreases slightly from the amplitude of the prior signal, creating a semi-synchronized sound effect only. Echo reverberation offers no utility value for behavior modification. In truth, the human brain does not reverberate nor does it think in onomatopoetic abstracts. Instead, the human brain resonates with bioelectrical impulses copiously defined in the prior art as brain waves, brain wave states, or brain wave frequencies, and additionally cited by Monroe in U.S. Pat. No. 3,884,218 (1978), and can be measured with an electroencephalograph. By echo reverberating the message, Taylor further dilutes the impact of the message by introducing an obfuscating abstract statement dissimilar to the patterns of normal human speech and cognitive reasoning. Echo reverberation in a round-robin fashion is the electronic equivalent of a jigsaw puzzle. By way of example, two individuals stand on opposing sides of the Grand Canyon and attempt to have a normal conversation. The refracted wavelength of the sound produces audibly torturous delays in the sound transmission and makes such a conversation, annoying, confusing, unintelligible, and useless. The left hemisphere of the brain will filter and restrict incoming communication to limit the excessive neuronal activity. Finally, Taylor records messages on the second channel backwards-in what Taylor defined as “metacontrast.” Taylor claimed that messages played backwards appear to excite the emotions in the right hemisphere of the brain. Indeed, any sound that receives conscious attention will induce excitation in one or both hemispheres of the brain, such as a baby crying or tires screeching on pavement or a bee bustling around one's head; each of which will produce an immediate response, neither of which the brain receives backwards. Taylor's approach is excessive, unnecessary, and refutable according to Becker who used two simple statements recorded in the normal forward method, both of which, to some extent, influenced the left brain hemisphere to recall memories from the right brain hemisphere of some store employees and some customers. Taylor used the ambiguous term “emotions” to define bioelectric activity in the right hemisphere of the brain. Emotions themselves are merely constructs, that without an underlying permanent memory would be nothing more than fleeting fantasy. Essentially, emotions are embodiments of neurons relating to some thing or some one, and recorded to permanent recallable memory. Taylor diminishes any plausible benefit of the messages because Taylor presents the messages as a cryptogram to the right hemisphere of the brain, which results in the creation of incomplete neuronal memory blocks, thus severely delaying interpretation and recall of the messages. Chai, in U.S. Pat. No. 6,615,197 B1 (2003) asserts that neuronal noise (excessive neuronal memory blocks) is a factor in senility, Alzheimer's disease, dyslexia, acaculia, and Attention Deficit Hyperactivity Disorders (ADHD). Consequently, the “whole brain” approach and the “round-robin” method depicted in the prior art promote mnemonic indifference, excessive neuronal noise, possible anxiety, and questionable utility value to users. Other sub-threshold methods and apparatuses are exemplified in U.S. Pat. Nos. 5,224,864 to Vavagiakis (1993), 5,245,666 to Mikell (1993), 5,270,800 to sweet (1993), 5,352,181 to Davis (1994), 5,827,071 to Sorensen et al. (1998), and 6,210,170 B1 to Sorensen et al. (2001), the teachings of each incorporated herein by reference.

While these methods and devices attempt to fulfill their respective, particular objectives and requirements, the aforementioned patents do not describe a method for quantum memory fusion. Furthermore, the prior art does not teach any known methods for quantum memory fusion. In these respects, the present invention substantially departs from the conventional concepts and designs of the prior art, and in so doing provides a method primarily developed for the purpose of quantum memory fusion that results in behavior modification or neuromuscular response or cognizant recitation response of data recorded or fused to permanent recallable memory, via electrical impulses introduced to certain sensory input receptors or a combination or plurality of input receptors simultaneously, including but not limited to the auditory canals or the ocular senses, or the auditory canals and the ocular senses of humans or animals.

### OBJECTS AND ADVANTAGES

Accordingly, it is one object of my invention to provide an all-natural, universally applicable method for writing data into and retrieving data from the memory of humans and animals.

Dr. Frederick Mitchell, a computer scientist and software engineer hereby introduces the terms, universal Human Technology, Molecular Memory, and Quantum Memory Fusion. Quantum Memory Fusion defines a process that occurs when data streams are audibly, visually, or tactilely recorded onto permanent recallable memory in such a manner that; a) the user believes that he or she has always known the data, b) the data is retrievable in its original form, and c) the data is permanent unless erased by Quantum Memory Fusion. For the sake of brevity, I use the words “fuse” or “fused” to reference the process above. Universal Human Technology is a first in science, because Molecular Memory and Quantum Memory Fusion describe a process applicable to the neurons in all of the 6.5 billion brains on the planet. Quantum Memory Fusion can be accomplished with auricular input only, ocular input only, auricular and ocular input, or other tactile sensory input, or any combination of sensory inputs. Quantum Memory Fusion can fuse an unlimited amount of information onto permanent recallable memory faster than any of the known traditional learning processes by creating Molecular Memory that can be addressed by its name for immediate recitation of data. Hereafter, molecular memory is interchangeable with permanent recallable memory.

There are many important sciences in the world. Neuroscience is the only science that focuses all human knowledge, from all areas of medicine and technology on a single subject—the study of the mind of man. Dr. Mitchell observed a pragmatic correlation between the computer sciences and what other scientists were attempting to achieve in neuroscience. This insight was in part the catalyst for Quantum Memory Fusion. Quantum Memory Fusion is the universal Human Technology poised as the foundation of neuroscience because it alone provides the uncomplicated transport mechanism and access into human memory by safely targeting molecular memory at and below the neuronal level, tapping into the near unlimited storage capacity of the brain.

In the more than ten thousand years of documented civilization, humankind has searched for ways and methods to access the raw power and immense storage capacity of the human brain. Unfortunately, the treatise, books, and articles written since 1710 are saturated with intriguing intellectual theory and presuppositions and therefore led humankind on a three-century pursuit for answers. In 1921 Bertrand Russell wrote “The Analysis of Mind” that shed some light on the mind of humans, but scarcely came close to solving the enigma of the mind. Sigmund Freud gave psychology further insight into the human mind. Until the 1950's, previous theories could not even benefit from advances in technology. However, in 1979, Professor Hal Becker from Toulane University performed an experiment that spurred an international obsession with abstract media. His experiment created an industry that touted self-improvement through abstract messaging, but without knowledge of how abstract messages actually work or why they ever produced any result or why the results are mostly failures.

Starting in the late 1980's many purveyors of memory enhancement products and abstract media flooded the consumer market with tricks and gimmicks to help one remember or modify one's behavior. These products met with great scrutiny, as consumers demonstrated considerable discernment about the use of such products in their daily lives. In part, the prudent consumer expressed anxiety about the content and the mysterious secret nature of abstract programs. Product manufacturers could not substantiate claims because no result could proven to be the result of the abstract media. Major retailers refused to stock or sell such products. Until the present invention, man has lived in abstract mnemonic darkness with no safe way to add, edit, or update memories on a voluminous scale.

Quantum Memory Fusion proves that the human brain is, as some scientists postulate, infinite in capacity. Quantum Memory Fusion disproves every known theory or treatise on the subject of abstract media at the supra and sub-threshold, and breathes new life into man's understanding of the power of the brain and memory.

Another object of my invention is to reduce the learning curve frequently encountered by practitioners that use new and innovative inventions. Quantum Memory Fusion incorporates an understandable set of terms that help to describe its components, features, and results, whereas the prior art relies phrases and terms too common for any practical understanding or perception. The language and definitions are the de facto standard for Quantum Memory Fusion and future technological advancement, although they are not to be construed as limiting. Table 1-1 below describes the Quantum Memory Fusion terms and definitions.

TABLE 1-1
Quantum Memory Fusion Terms, Abbreviations and Definitions

| | Term | Abbr. | Description |
|---|---|---|---|
| 1. | Abstract Data Stream | ADS | Secondary audio or video stream |
| | | | transmitted sub-threshold. |
| 2. | Action Query | AQ | Questions designed to elicit incognizant |
| | | | or cognizant behavioral response or |
| | | | cognizant neuromuscular response in |
| | | | reply to opposing or self-induced |
| | | | motions or movements |
| 3. | Bio-electro-harmonic | BEH | The assembled biochemical equivalent |
| | | | of human language that resonates as |
| | | | output from the harmonic synaptic cleft. |
| 4. | Basic Operating System | BOS | The operating system of the right |
| | | | hemisphere of the brain. The BOS |
| | | | communicates within this region using |
| | | | Organic Machine Language |
| 5 | Cognizant Neuromuscular | CNMR | The physical body movements |
| | Response | | demonstrated by an individual because |
| | | | of data fused to memory. |
| 6. | Cognizant Operating System | COS | The operating system of the left |
| | | | hemisphere of the brain. The COS |
| | | | communicates within this region, and |
| | | | with the right hemisphere of the brain, in |
| | | | the native language of the individual. |
| 7. | Cognizant Quantum Index | CQI | The tactile data stream consisting of the |
| | | | primary index and the solution index. |
| | | | The CQI may also be incognizant. |
| 8. | Cognizant Recitation | CRR | The ability to recite upon query, data |
| | Response | | fused to permanent recallable memory. |
| 9. | Dendritic Axonic Harmonic | DAHO | The bioelectrical alphanumeric output at |
| | Output | | the presynaptic terminals of the Organic |
| | | | Machine Language Interpreter. |
| 10. | Dendritic Data Directory | DDD | The theoretical location of data storage |
| | | | on a neuron. |
| 11. | Diamond Data Flow | DDF | The direction of travel of data |
| | | | transmissions in the neuronal memory |
| | | | block. The DDF has purpose and utility |
| | | | to facilitate interconnectivity from other |
| | | | neuronal memory blocks. |
| 12. | Data Stream Stacking | DSS | The process of creating multiple |
| | | | synchronized abstract streams of the |
| | | | same or different information that varies |
| | | | in amplitude, decibel volume, pitch, or |
| | | | frequency. |
| 13. | Filter Data Dendrite | FDD | First dendrite on the query neuron that |
| | | | receives information. |
| 14. | Filter Index Nucleus | FID | The most important part of the solution |
| | | | neuron. It is not necessary that this |
| | | | nucleus share the same index with the |
| | | | primary neuron or the solution neuron. |
| 15. | Filter Key Dendrite | FKD | The dendrite on the query neuron that |
| | | | contains some connection to the PIN, |
| | | | SIN, and FIN (although not absolutely |
| | | | necessary that a relationship exists) |
| 16. | Filter Query Soma | FQS | Contains the name of the query neuron |
| 17. | FusionWare ™ | FW | The programs, documentation, and |
| | | | transports of molecular memory fusion. |
| 18. | Harmonic Synaptic Cleft | HSC | The microscopic space between the |
| | | | presynaptic terminals of the Organic |
| | | | Machine Language Interpreter and the |
| | | | dendrites of neurons from the neural |
| | | | network, that relay bio-electro-harmonic |
| | | | impulses to the speech center of the |
| | | | brain. |
| 19. | Incognizant Behavior | IBC | The behavior model fused to memory |
| | Construct | | that creates a change in cognitive |
| | | | activity based upon internal or external |
| | | | stimulus. |
| 20. | Incognizant Personality | IPC | The personality model fused to memory |
| | Construct | | that creates a change in cognitive |
| | | | methods based upon the methods of |
| | | | one or more role models. |
| 21. | Kelly's Query Funnel | KQF | The presupposition that if a plurality of |
| | | | related topics were fused onto memory, |
| | | | followed by an unanswerable query, that |
| | | | the resulting solution would be |
| | | | equivalent to genius savantism. |
| 22. | Language to Language | LLT | The neuron or neuronal memory block in |
| | Translator | | the left hemisphere of the brain that |
| | | | converts native language to another |
| | | | language in a multi-lingual brain. |
| 23. | Molecular Memory | MM | The microscopically rearranged sub- |
| | | | cellular data in a neuronal memory block. |
| 24. | Neuro Critical Mass | NCM | The state of a neuron when it discharges |
| | | | its potential |
| 25. | Neural Data Streaming | NDS | The processing of locking a dendrite into |
| | | | a continuous stream of audio, visual or |
| | | | tactile data. |
| 26. | Neuronal Memory Block | NMB | The group of neurons that contain the |
| | | | organic machine language form of a core |
| | | | memory, including two organic machine |
| | | | language interpreter neurons. |
| 27. | Neuro Nucleo Dendritic | NNDT | The naming of a neuron, nucleus and one |
| | Targeting | | or more of its dendrites. |
| 28. | Named Neuronal Registry | NNR | The neuron that contains specific data |
| | | | and environmental variables that describe |
| | | | the memory contained in its neuronal |
| | | | memory block. |
| 29. | Neurothreading | NT | The process of transmitting multiple |
| | | | instances of similar or different streams of |
| | | | audible, visual or tactile data to neurons. |
| 30. | Organic Machine Language | OML | The biochemical, bioelectrical language of |
| | | | all brains. OML is either “1” or “0” or both. |
| 31. | OMLI Neuron | — | The neuron of a neuronal memory block |
| | | | that converts organic machine language |
| | | | to the native language of that brain or |
| | | | native language to organic machine |
| | | | language. |
| 32. | Organic Machine Language | OMLI | The neuron or neuronal memory block |
| | Interpreter | | that converts organic machine language |
| | | | to the native language of that brain or |
| | | | native language to organic machine |
| | | | language. |
| 33. | Primary Data Dendrite | PDD | First dendrite to receive information in the |
| | | | primary index |
| 34. | Primary Index | PI | The first four-block of data in the quantum |
| | | | index. |
| 35. | Primary Index Nucleus | PIN | The most important part of the primary |
| | | | neuron because it contains the same |
| | | | index. |
| 36. | Primary Key Dendrite | PKD | The dendrite that maintains some |
| | | | connection to the PIN (although not |
| | | | absolutely necessary that a relationship |
| | | | exists) |
| 37. | Primary Neuron | PN | Third neuron in a diamond-shaped |
| | | | neuronal memory block. |
| 38. | Primary Ordinal Soma | POS | Numbered neuron that contains data |
| | | | relevant to the core memory. |
| 39. | Quantum Data Filter | QDF | The neuron(s) that that challenges the |
| | | | neuronal memory block to return only |
| | | | the correct response. |
| 40. | Quantum Fusion Formula | QFF | Mathematical equations that determine |
| | | | the fusion constants |
| 41. | Quantum Memory Fusion | QMF | The process of recording retrievable |
| | | | data to the brain at the neuronal level. |
| 42. | Query Neuron | QN | Contains one or more queries against |
| | | | other neurons in the neuronal memory |
| | | | block. |
| 43. | Registry Data Dendrite | RDD | First dendrite to receive data or |
| | | | environmental variables |
| 44. | Registry Index Nucleus | RIN | The RIN may or may not contain data |
| | | | that specifically relates the PIN, FIN, |
| | | | and SIN. The RIN may serve as a |
| | | | cataloging tool or a group data |
| | | | reference tool. |
| 45. | Registry Key Dendrite | RKD | A dendrite that may contain some |
| | | | connection to the PIN, SIN, and FIN |
| | | | (although not absolutely necessary that |
| | | | a relationship exists). |
| 46. | Reflexive Memory Recall | RMR | An internal query initiated by the |
| | | | conscious or subconscious actions of an |
| | | | individual based on the presence of a |
| | | | query neuron. |
| 47. | Registry Neuron | RN | Second neuron in a diamond-shaped |
| | | | neuronal memory block and responsible |
| | | | for recording session information and |
| | | | environmental variables |
| 48. | Registry Name Soma | RNS | Name of the first programmable |
| 49. | Recitation Query | RQ | Questions that evoke a verbal solution, |
| | | | or cognizant recitation response to non- |
| | | | physical stimulus. |
| 50. | Solution Data Dendrite | SDS | First dendrite on the fourth downstream |
| | | | neuron to receive information |
| 51. | Solution Index | SI | The second four-block of data in the |
| | | | quantum index |
| 52. | Solution Index Nucleus | SIN | The most important part of the solution |
| | | | neuron that contains the index |
| 53. | Solution Key Dendrite | SKD | The dendrite that contains some |
| | | | connection to the PIN and the SIN |
| | | | (although not absolutely necessary that |
| | | | a relationship exists) |
| 54. | Sub Mnemonic | SMCP | The format of incognizant data that |
| | Communications Protocol | | queries memory using the Quantum |
| | | | Data Filter. |
| 55. | Sub Mnemonic Query | SMQ | Sub-threshold abstract questions |
| 56. | Solution Main Soma | SMS | Name of the fourth downstream neuron |
| | | | that stores the solution |
| 57. | Solution Neuron | SN | Records the answers to the primary |
| | | | neuron |
| 58. | Tactile Data Stream | TDS | Liminal audio or video stream. |
| 59. | Universal Human Technology | UHT | Biotechnology mainly applicable to |
| | | | Homosapiens but may serve other |
| | | | homogeneous species. |

Another object of my invention is to provide a means of fusing memories in named neurons of the brain.

Still another object of my invention is to retrieve memories from named neurons.

Yet another object of my invention is to erase fused memories after the memory is no longer needed or requires updating or editing.

In addition to the above objects and advantages, several additional objects and advantages of the present invention are described in the following Factors 1.a. to 1.n., 2.a. to 2.o, 3.a. to 3.x, and 4.a. to 4.d. below.
1.a. Neurons are organic computers. The human brain is a repository of billions of these organic, microscopic devices. Popular scientific theory hypothesizes that the brain is like a computer. Although there are numerous similarities, this theory is misleading. However, there are many more similarities between a single neuron and a computer. The system registry, permanent storage, and other devices theoretically exist in both the inorganic personal computer and the organic neuronal computer. The neuron is complete in that it contains all of the organic devices necessary to perform its functions and assist other neurons in performing their functions. Factors 1.a.1-1.a.11 compares the similarities of neurons to computers.
   1.a.1. Somas compare to named system registries.
   1.a.2. Dendrites compare to Erasable Programmable Read only Memory (EPROM).
   1.a.3. Nuclei compare to CPU and router.
   1.a.4. Axons compare to serial cable.
   1.a.5. Myelin sheaths compare to network hubs.
   1.a.6. Neurilemma Cells compare to power supplys.
   1.a.7. Nodes of Ranvier compare to network ports.
   1.a.8. vesicles compare to network data packets.
   1.a.9. Neurotransmitters compare to network data.
   1.a.10. Terminals compare to serial male connectors.
   1.a.11. Receptor molecules compare to serial ports.
1.b. The human brain has dual operating systems. The two systems are the Cognizant Operating System (COS) and the Basic operating System (BOS). The COS is merely one's self. We communicate with the COS in our native language. All of our internal thoughts terminate in the COS. The BOS controls all other functions to include the memory. The BOS communicates in “organic Machine Language” (OML). What is amazing is that the COS and the BOS communicate in the native language of the individual. This led my research to conclude that in the right hemisphere of the brain exists another neuron that acts as an “organic Machine Language Interpreter” (OMLI). The OMLI is part of the neuronal memory block. This neuron receives the OML output from within the neuronal memory block, converts this data to native language, and finally dumps the buffer across a neural network relay that transmits to the speech centers of the brain. Among the 100 billion neurons, there could be hundreds of millions of neurons that act as OMLI's for past, present and future memories, or any neuron can become an OMLI. Then, there is an even more amazing discovery about the OMLI. The OMLI can not interpret language data types. That is, the language by which the memory was recorded is the only language that an OMLI in the neuronal memory block will output. For persons that learn additional languages, the memories stored in their native language will always output in their native language. Memories stored in another language will always output in that language. This discovery led to yet another discovery, and that is the existence of a “Language to Language Translator” (LLT). The LLT exists in the right hemisphere just after the neural network relay and before the speech center. This stands to reason because we always “think” in our native language even if we are multilingual. Memories are stored in much the same manner as computer data is stored to a hard drive. The data in one form represents our literal interpretations such as the viewing and recognition of text or images. In its most basic form called machine language, the data represents a series of “1's” and “0's.” Before a literal and understandable representation of the data is consciously available, the machine language must be translated. Neurons biochemically communicate in organic machine language, not literal representations. Using an electroencephalograph, we can see the electrical impulses associated with the occurrence of a memory, it is impossible to decipher what the series of electrical impulses represent as far as data. The final evidence is that there are only two states, the action potential and the resting potential. This is the language of the brain. The theory that memories can be encoded by the individual through tricks or gimmicks is false. The individual provides only the peripheral sensors for data input and intake that creates other unnamed neuronal connections. Until the present invention, how and where the data was stored was outside the control of the individual.
1.c. Quantum Memory Fusion organizes memories. The greatest revelation in the organic computer theory is that all of the similarities between the personal computer and the human brain are available in each of the 100 billion or more neurons. That is, each neuron is a completely independent computing system that communicates with other neurons in a neural network of incomprehensible magnitude. The neural network can change its configuration at will. Unlike popular theory, the configuration of the neural network is not random or arbitrary. Instead, the configuration changes as the direct result of new data and relates to how that data will be stored and retrieved. Quantum Memory Fusion encourages neural network configuration changes and promotes healthy and organized memory fusion and recall.
1.d. Quantum Memory Fusion targets the neuron. Dr. Frederick Mitchell introduced the term “Neuro Nucleo Dendritic Targeting” (NNDT) to describe the method of naming and communicating with neurons at the sub-mnemonic level. Memories are stored in the dendrites, not in a fluid between cells. Popular scientific theory states that memories are either stored in the synaptic cleft or not stored at all, just “found”. That popular theory is impossible to substantiate for a host of reasons. First, the chemical composition of neurotransmissions creates a magnetic field at the synaptic cleft. This magnetic field is also an unshielded electrical circuit pulsating with alternating current. The volatile nature of the synaptic cleft would constantly erase and distort data in neighboring synapses thousands of times per second. Next, Magnetic Resonance Imaging (MRI) would literally demagnetize the vesicles, neurotransmitters, and every synaptic cleft in the brain. My invention indicates that memories are stored in the abundance of dendrites located on each neuron. Quantum Memory Fusion names the neuron, nucleus, and dendrites that specifically store data onto neuronal memory blocks.
1.c. Quantum Fusion Formulas determine how much data can be fused onto memory and the time required for fusion to occur. The formulas are more adequately described in Factors 1.c.1-1.c.2.
   1.c.1. For a child up to 12 years old and with full cognitive abilities, my formula states that 15 items can be fused to memory in sixty minutes using audio input only. The formula ((60/12)×3) is the Quantum Fusion Formula for children. This figure could quadruple when images are fused with associated data strings.
   1.c.2. For an adult with full cognitive abilities, our formula states that 30 items can be fused to memory in sixty minutes using audio input only. The formula (((60/12)×3)×2) is the Quantum Fusion Formula for adults. This figure could quadruple when images are fused with associated data strings.
1.d. Quantum Fusion Formulas can be affected by four variables as described in Factors 1.d.1-1.d.2 below.
   1.d.1. The first variable is concentration. Our experiments show empirical evidence of an increase in fusion rate when the test subject concentrates on the data presented. It is easier to fuse data that the test subject desires. I noted that concentration and desire may not necessarily be interconnected, but that the possible reward of having the data fused onto memory (success, more knowledge) increases absorption.
   1.d.2. The second variable is age. The older test subjects seemed more susceptible to fusion. Therefore, it was possible to fuse two to three times the amount of data in the same sixty-minute session. I attribute this phenomenon to maturity and attention span.
   1.d.3. The third variable is familiarity. Data that is days, weeks, or months old can be fused onto memory without the test subject hearing or seeing any of the data during a fusion session. Test evidence from numerous experiments supports this variables
   1.d.4. The fourth variable is visual data. Visual data fuses faster. When visual data and audible data exist for the same topic, fusion occurs in less time and produces a higher response rate because of dual sensory input. Apparently, ocular sensory perception is of great value.
1.e. Quantum Fusion Formulas force neurons to reach Neuro Critical Mass.
1.f. Quantum Memory Fusion produces tangible results in sixty minutes or less. In numerous experiments, test subjects learned new and unfamiliar data that would normally have taken months or years to learn. In one audio fusion experiment, a seven year-old learned the multiplication facts of 12 in approximately sixty minutes. Five days later, when asked random multiplication facts of 12, the test subject recited the answers without hesitation. There had been no other testing or studying permitted.
1.g. Quantum Memory Fusion records data to memory in a method that makes the memory permanently recallable. Referring back to 1.e above, the test subject stated that it felt as though she had always known her multiplication facts of twelve. In another one-hour audio fusion experiment, an adult test subject was tasked with learning the names of inventors and their inventions. Of the 41 items fused to the test subjects' memory, the test subject recited 34 of them with precision, representing an 82% accuracy rate. The test subject was able to answer questions asked in random order or by its ordinal position. The following day, the test subject remembered two additional answers from the list without prompting, increasing the score to 87%. The test subject did not interact with the data after the initial test. The two additional answers were advantageous side effects of the fusion process. It is important to note that each line of data consists of 12 to 16 data strings, of which only one is the answer. Therefore, to recite 34 items correctly on the first attempt is to have remembered 408 to 544 items in one hour. There were no visual clues or interactions with the data after the experiment concluded. Interestingly, the data that did not fuse created a literal “void” in the test subject's memory where an answer would normally reside, in that even a guess was grossly inaccurate. This supports my theory that Quantum Memory Fusion initiates Neuro critical Mass. If a neuronal memory block fails to reach Neuro critical Mass, no data records. This is why traditional methods of learning are slow.
1.h. Quantum Memory Fusion uses unobvious methods that create new and unexpected results. Dr. Frederick Mitchell introduced the term “Quantum Data Filter” (QDF) to describe the process of creating a query structure that forces the memory to reassemble data before an external query against the data is applied. The Quantum Data Filter queries the data during fusion. These constant queries force the neuronal memory block to respond even while the data is inbound. The primary purpose of the QDF is to eliminate all other possible responses except for the correct response. A very advantageous side effect of the filter is that if the filter is externally queried by its neuronal name and if the test subject can not immediate recite the answer, the Quantum Data Filter will produce its own sub Mnemonic Query (SMQ), against the neuronal memory block. In 98% of experiments that used the Quantum Data Filter, all test subjects reported hearing a harmonic voice that consciously spoke the answer, usually within five minutes. This harmonic voice appeared to originate at the right hemisphere and travel upwards and leftward to the left hemisphere of the brain. The voice appeared to be stereophonic although it originated in only one half of the brain. The harmonic voice remained stereophonic as it panned leftward. It was at the leftmost position that the stereophonic words in the harmonic voice could be consciously spoken. This action helps support my theory of the organic Machine Language Interpreter. In one experiment a test subject dreamt the fused answer and recited the answer the following day. The Quantum Data Filter is not required to produce the results of Quantum Memory Fusion. However, the QDF enhances one's own mental capacity to solve problems and locate information in the cache of knowledge of that particular brain.
1.i. Quantum Memory Fusion is duplicable. The QFF's are applicable to all cognizant persons. My methods will produce the same measurable results regardless of who the test subject is or where the test subject resides.
1.j. Quantum Memory Fusion can be applied to every known subject matter and placed on all known forms of media. Products designed with Quantum Memory Fusion and the Quantum Fusion Formulas can fit on a single computer disk, memory card or compact disk. Thus, products utilizing QMF are small, portable, inexpensive, and easy to build. File sizes for audible data with up to 15 data streams can be as small as 20 megabytes, which makes web page delivery fast, inexpensive, and reusable. Furthermore, manufacturers can apply Quantum Memory Fusion in a purely written format for easy to learn text. Manufacturers can deliver Quantum Memory Fusion programs across the internet, an intranet, satellite, and cable television, and through radio broadcasts. Quantum Memory Fusion can target the neurons of any human or animal within proximity of its a transmission.
1.k. Quantum Memory Fusion fully utilizes the massive storage capacity of the right hemisphere of the brain. A virtually unlimited amount of retrievable data can be fused to the more than 100 billion neurons in the human brain; with the only limit being the number of available neuronal memory blocks. As my invention will later prove, a neuronal memory block consists of a minimum of six neurons formed in a diamond shape. Each neuron can theoretically store up to one hundred and twenty thousand words, each word stored in a separate dendrite. Based upon the number of dendrites on each neuron, a neuronal memory block could theoretically store 720,000 (seven hundred twenty thousand) words. Therefore, by my modest estimation, there are approximately 17,000,000,000 (seventeen billion) neuronal memory blocks available in a human brain, each of which can store 720,000 eight-character words. Therefore, I assert that the human brain can store approximately 12,240,000,000,000,000 (twelve quadrillion, two hundred forty trillion) words or 97,920,000,000,000,000 (ninety seven quadrillion nine hundred twenty trillion) individual characters or numbers—approximately. This means that humans use only a small fraction of their brains throughout their lifetimes.
1.l. Neuro Nucleo Dendritic Targeting permits naming of the “Dendritic Data Directory” (DDD).
1.m. Because Quantum Memory Fusion uses NNDT, previously fused data can be erased from memory although other neurons have attached to the memory block and created extraneous synaptic connections that QMF can not undo. The unexpected result is that the fused memory is erased and the newly attached neurons are incapable of recreating the deleted memory.
1.n. In much the same manner, NNDT allows Quantum Memory Fusion users to update previously fused memories by re-writing the original data with new or modified data.

### ADDITIONAL OBJECTS AND ADVANTAGES

2.a. Quantum Memory Fusion solves the problems with abstract sub-threshold media as described in Factors 2.a.-2.o., while Factors 2.a.1.-2.a.4. provide additional answers.
   2.a.1. The abstract message myth. The long standing theory behind abstract content is that if an abstract suggestion or message is introduced to a person below the sub-threshold, that the person will “mystically” absorb this information and later act out the message in some form of behavioral modification. Three important events fueled these myths as depicted in Factors 2.a.1.a.-2.a.1.c. below.
      2.a.1.a. In the 1950's, the flashing of a picture of popcorn during a film that purportedly increases sales of popcorn during intermission at a movie theatre.
      2.a.1.b. In 1979 Becker broadcasts two abstract messages “Thou shall not steal” and “Honesty is the best policy” throughout a New Orleans supermarket and purportedly reduces the cash shortages, inventory damage, and pilfering.
   2.a.1.c. in 1993, two young men attempted suicide. Both men listened to music that contained an abstract message stating “Do it.” Based upon this message, the two men decided to take their own lives. One young man shot himself and died instantly. The other fatally wounded himself and died three years later.
   2.a.2. The abstract, sub-threshold message truth. Dr. Frederick Mitchell began the creation of a treatise that explains this phenomenon. In order to understand the truth about abstract messages, we must set aside the message and revisit the neuron. As previously discussed, the neuron has two states, the action potential or NCM and the resting potential. For data entering the human senses to create Neuro critical Mass, it must meet one of two qualification as shown in Factors 2.a.2.a-2.a.2.b below.
      2.a.2.a. The data must be cognizant now. That is, it can not be a fantasy or phantom construct that fails to register on the sensory receptors. The sensations of touch, taste, smell, site, and hearing all produce cognizant data.
      2.a.2.b. The data must already exist in the cache of knowledge of that brain. This cache includes all known events that occurred during the lifetime of that brain and all knowledge accumulated by that brain; recent data being of utmost value.

2.a.3. if you present a query to the left hemisphere, the right hemisphere must answer the query. It is important to note that the query can be in the form of an actual question or the query could be an environmental stimulus that elicits a certain behavior. If the “Named Neuronal Registry” (NNR) can locate substantiated data, then it will force Neuro critical Mass that signals the primary neuron to either begin recording new data or delivering existing data to the OMLI. If the NNR finds nothing to support the data, then the Neuronal Memory Block rests. This failed recovery forces the OMLI to issue a plain language message directly to the cognitive left hemisphere in response to the original query. Table 2-1 illustrates plausible OMLI responses.

TABLE 2-1
Organic Machine Language Interpreter Reply to Query

| | | | OMLI Plain | |
|---|---|---|---|---|
| NNR | NMB | | Language | |
| Output | Output | Results | Reply | Cognitive Output |
| 1 | 1 | Memory | YES | Action Taken |
| | | Found | | (Retrieve, Interpret, |
| | | | | Output) |
| | | | | “The answer is . . . ” |
| 0 | 1 | Memory | SOUNDS | Action Anticipated |
| | | Fragmented | LIKE . . . | (Retry) |
| | | | | “It's on the tip of my |
| | | | | tongue.” |
| 0 | 0 | Memory | NO | Action Cancelled |
| | | Not Found | | (Quit, Reset, Rest) |
| | | | | “I can't remember.” |

2.a.4. Returning to the issue of the abstract message. If the message “I am a good person” is injected at the sub-threshold of an adult that has recently displayed incorrigible behavior, this message must record to memory first. The only way that it will record to memory is defined in 2.a.2.a above. Additionally, the message must be persuasive, repetitive, or delivered at the right developmental stage to attract other neuronal memory blocks. In this case, persuasion and repetition are easy to produce, while we assume that the right developmental stage is that critical juncture in the life of this adult versus at a certain age. Assuming that at some time in the life of this adult, he or she was in fact a “good” person, then in the cache of knowledge of that brain, concrete evidence exists that converts this fantasy, phantom construct to pseudo-cognizant data. Such evidence could consist of an encouraging comment to a friend, donating money to a charity, giving food to a homeless person, etc. The message is eligible for recordation and incognizant behavioral modification because confirmation of existence of the data produced an answerable query that returned “1,1,YES, Action” as shown in Table 2-1. We can apply this theorem to the popcorn sales. Because everyone in the theatre had at some time seen, smelled, tasted or even popped popcorn, the image of popcorn on the screen produced an answerable query that returned “1,1,YES, Action” as shown in Table 2-1. Applying the same theory to the Becker experiment, nearly all people have heard the phrases “Thou shall not steal” and “Honesty is the best policy.” whether from parents in the home, on television, or in some religious setting, these are inescapable phrases of our ancestry. Thus, those abstract messages were pseudo-cognizant and already stored in the cache of knowledge of those brains affected. Since this is true, why did any cash shortages, inventory damage, and pilfering continue? The reasons are simple. First, it is possible that not all patrons spoke English. French is another language spoken in New Orleans. Becker did not state whether he recorded the messages in other languages. A French speaking person thinks in French. If the patron did not speak English, there would be no query at all and Table 2-1 is inapplicable. Next, store personnel that worked in areas of the store not susceptible to the message would be unaware of its existence; again, Table 2-1 is inapplicable. Finally, patrons or personnel that believed contrary to the content of the messages could not be influenced because the answerable query returned “0,0,NO, cancel” as shown in Table 2-1. Abstract messages are not mystical; they can be explained with scientific observation. Supported by my research and experiments, I announce that this is why abstract, below supra and sub-threshold messages produce or fail to produce results. Because of my findings, the current definition of the word “sub” combined with the word “limen” is improbable. The American Heritage Dictionary—Fourth Edition 2000, defines it as “Inadequate to produce conscious awareness but able to evoke a response.” First, nothing will produce conscious awareness if the “awareness” was not at some time in the past conscious see 2.a.2.a and 2.a.2.b above. Next, after more than seventy experiments spanning 10 years, and more than two hundred tests of varying degrees of mnemonic difficulty, I can write with certainty that a fantasy or phantom construct will not produce a tangible response if the fantasy or phantom construct can not locate substantive evidence that it or something that resembles it exists in permanent recallable memory. If my theories were untrue, then humans could manifest dreams into reality and bring from within a dream tangible evidence of the dream. I can safely define the words “sub” and the suffix word “limen” as “Adequate to produce conscious awareness and able to evoke a response if stimulus pertaining to that awareness or some variation of that awareness exists in the permanent memory of the individual.”
2.b. Quantum Memory Fusion can force on-demand Neuro critical Mass. It is well established that a memory recalls or records when a neuron reaches Neuro critical Mass and discharges it potential. See Factors 2.b.1-2.b.7 below.
   2.b.1. My research proves that humans remember everything they “hear” or “see” whenever it commands attention. However, the events are unendorsed by other neuronal memory blocks and too disorganized to recall. Nevertheless, to understand why so little of what we experience is “recallable,” we must take leave to discuss the memory structure itself. Thus far, no scientific theory has even postulated a design for memory. Inasmuch, the understanding of mnemonic workings is limited. What is thought to be understood by other scientists is lacking in supporting evidence.
   2.b.2. Events arrive at our sensory receptors; our memory stores these events in the order received. According to Quantum Memory Fusion results, the events are completely out of order. The brain thrives on order. Quantum Fusion Formulas are byproduct discoveries of this order. The first part of this order is the “shape” of a memory. I assert that memories are stored in neuronal memory blocks. These neuronal memory blocks consist of no less than six neurons theoretically arranged in the shape of a diamond. This diamond shape is the perfect design for electrical conductivity because of the organized Diamond Data Flow and because the shape allows additional diamond-shaped neuronal memory blocks to easily and unobtrusively attach to each other on a plurality of diamond-shaped sides. Each neuron in the diamond has four data input blocks. The data input blocks represent the individual strings of data that constitute a portion of a complete memory. The first neuron sits at the apex of the diamond. This neuron is an organic Machine Language Interpreter. Its only function is to convert incoming events to bioelectrical impulses that represent the biochemical equivalent of the event. I termed this biochemical equivalent “organic Machine Language.” The second neuron sits on the left most side of the diamond (as viewed from a one-dimensional perspective). This is the Named Neuronal Registry neuron. Its main function is to provide the environmental variables about a memory that help to produce the “feeling” portion of a memory and the identity of a memory. Information such as time, date, age, location, mood, body temperature, name of best friend, etc., are environmental variables, which themselves are randomly selected thoughts and images beyond the control of the person. This is the absolute only encoding that will ever occur in the brain, and it is not really encoding at all, it is more of a mnemonic security measure. This security method creates an explicit neuronal memory block that no other neuronal memory block can duplicate when making synaptic connections to the registry neuron. The Named Neuronal Registry neuron is the only neuron in the diamond that records illogical data as part of its function. I discovered that the registry neuron further records the ongoing feelings associated with the memory. Essentially, the registry neuron will track and record all feelings produced within that neuronal memory block and provide security for the data. Immediately right of the NNR is the third neuron, the Primary Neuron. The primary neuron is the key to the known portions of recallable memories within neuronal memory blocks. The primary neuron contains, at the very least, an ordinal position in the first block. In the fourth block is the data the makes the primary neuron the most important—the index. The fourth neuron is the solution Neuron. Its function is to provide the answer to the yet to be asked question. It contains the same index in its fourth block, as does the primary neuron. The fifth neuron is the Query Neuron. The query neuron has an interesting function in that it only seeks solutions. It too has as its index the same index as the solution neuron and the primary neuron, although not specifically required. These three neurons must relate to each other in order to locate data stored on dendrites in either. The sixth and final neuron is another organic machine language interpreter neuron. It converts the organic machine language of the neuronal memory block back to the native language spoken by the individual. This discovery was unique in that the neuron performed independently of other suspected neurons, which led me to its existence.
   2.b.3 Now that we understand the design of memory, we return to the storing and retrieving of memories. When incoming events arrive at the neuronal memory block, the event will continue to write to a data block beyond the end of the primary neuron's index. That alone will make the memory useless for purposes of recall. Until the primary neuron has an index that the remaining neurons share and reference, there can be no memory recall. I solved this problem with a Natural Neuronal Buffer that consists of absolute silence of varying lengths. The NNB is equivalent to “end of file” in software engineering terms.
   2.b.4. The disorganized input from the event creates multiple unrelated neuronal memory blocks or just one neuronal memory block without an index. If more than one NMB is created by the long string of incoming events, relevant portions of the main event will spread across other NMB's creating Dendritic Data Directories. The DDD'S in other NMB's are unknown to the primary neuron in the first NMB. Thus, the data written to other DDD'S is permanent but not recallable. Because no primary index exist between the original neuronal memory block and subsequent neuronal memory blocks, they can not bind to make the permanent memory recallable. Essentially, the index is “the tie that binds” it all together.
   2.b.5. If the incoming events command attention and are properly organized and indexed, the events will be recorded to dendrites. However, that alone will not make the memory recallable, only permanent. Hence, the confusion of how memory works. A recallable memory has some specific requirements to be recallable, whereas a permanent memory has no requirements to become permanent. Every word, event, sound, sight, smell, touch and taste that has ever commanded even a modicum of attention is permanently stored in memory—forever.
   2.b.6. For a permanent memory to be recallable, it must be properly indexed, and at least one other neuron from a pre-existing neuronal memory block must attach to the newly formed neuronal memory block. This attachment is first an endorsement of the contents, and then an agreement that at least one bit of data in the new memory block has some value to the memory system (value being extremely subjective) AND that the dendrite containing this data resides on a neuron that can serve as a conduit for at least one other neuronal memory block in the brain. This stupendously perfect system guarantees the brain that its neural network will remain intact and that recallable memories will travel the shortest path to recitation.
   2.b.7. An example of the neuronal memory block to neuronal memory block relationship is when a person “remembers” an event that had no apparent value at the time it occurred. This event is stored in a “years-old NMB.” Because the event in the years-old NMB did not have value to the memory system at the time it happened years earlier, it could not form a relationship with another neuronal memory blocks. Thus, the years-old NMB was permanent, but not recallable. Then, a new event occurs and creates a new NMB that somehow needs or wants data that resides on one or more dendrites in the years-old NMB. The new NMB makes spontaneous synaptic connections to dendrites in the years-old NMB, and at that moment the person experiences a “flash” of the years-old NMB memory. This memory flash is the result of the 200-mph data-read-back of the contents of the years-old NMB. The data-read-back happens because every neuron is an independent organic computer responsible for its own connections to the neural network. NOW that the new NMB has found purpose in the years-old NMB and made a synaptic connection to a data dendrite, it will read the contents of every dendrite in the years-old NMB and make as many connections as possible at a speed of approximately 200-mph. Arguably, the new NMB wants prime position on all useful data in the years-old NMB because as in any network, the closer you are to the source of the data, the faster you can access the data. Then, the years-old NMB announces its contents to every other neuronal memory block in the brain by forcing its memory block to flush its contents across the neural network. However, the years-old NMB is the only neuronal memory block in which the new-found memory will ever reside. The years-old NMB then goes to work advertising its data stores to every possible neuronal memory block because each neuronal memory block is “self aware.” creating more connections to other NMB's guarantees the strength of the memory, memory recall speed and its own longevity. There is only one artificial system known to man that behaves in a similar manner and that is the artificial neural network commonly referred to as the internet.
2.c. Quantum Memory Fusion records data onto permanent recallable memory even when the subject objects to the data.
2.d. Quantum Memory Fusion accelerates the rate of learning.
2.e. A user can modify his or her behavior absent of psychotherapy intermediaries.
2.f. Quantum Memory Fusion requires no user training, no user intervention, and no third party interactions before the user receives the immediate benefit of the invention.
2.g. Quantum Memory Fusion causes no discomfort to the user.
2.h. Quantum Memory Fusion allows the user to modify his or her behavior quickly using uncomplicated spoken words.
2.i. Quantum Memory Fusion eliminates the need for hypnosis, post hypnotic suggestions, artificial environmental “triggers,” devices or apparatuses, to effect genuine and permanent behavior modification.
2.j. Quantum Memory Fusion enhances neurological responsiveness to external stimulus and queries.
2.k. Quantum Memory Fusion can record an “Incognizant Behavioral construct” (IBC) or “Incognizant Personality construct” (IPC) to permanent recallable memory that supersedes what the user regards as an undesirable behavior or personality traits.
2.1. Quantum Memory Fusion eliminates “neuronal noise” decreases response time and error rate because QMF communicates with a neuronal memory block at the sub mnemonic level.
2.m. Quantum Memory Fusion experiments repeatedly produced tangible, permanent, impressive, and painless results within sixty-minutes using a delivery method termed “Day Trainer.” with Day Trainers, the user is wide-awake and may even concentrate on other tasks during a sixty minute session.
2.n. Quantum Memory Fusion can be used as a “Sleep Trainer” for nighttime fusion. Sleep Trainer results have yielded a cognizant Recitation Response for data that the user experienced up to two years previously. Sleep Trainers helped to prove that information stored in the cache of knowledge of the brain can be “pulled” forward and reorganized under a completely new Neuronal Memory Block based on the query presented by the Quantum Data Filter. By introducing data to the user at the time primarily relegated to the sleep states, QMF capitalizes on the “always on” function of the auricular senses.
2.o. Quantum Memory Fusion is voluntary and ethical because the user will hear or see the data or both.

### MORE OBJECTS AND ADVANTAGES

3.a. Quantum Memory Fusion provides a means for psychotherapy intermediaries to serve their patients more efficiently and less costly because much of the data that would not be absorbed by patient over numerous therapy sessions can be fused to permanent recallable memory in sixty minutes for permanent improvement in the patient.
3.b. Quantum Memory Fusion can be used as a transport mechanism for diagnostic solutions recommended by psychiatric and psychotherapeutic practitioners for their patients, based on the evaluation of the patient by the practitioner.
3.c. Quantum Memory Fusion gives therapy professionals the first opportunity in the history to serve a global patient group. Diagnostic solutions can be delivered via internet web portals, voicemail, radio, television, even through wireless telephone transmissions of sufficient length. The usual one-to-one therapy session of sixty minutes can now be one-to-millions simultaneously in sixty minutes.
3.d. Quantum Memory Fusion can be used as a transport mechanism for confidential information. Delivering secure documents pose risks to the carrier and the data. With QMF the user fuses the important data onto memory, later recites the data upon query, and then erases the data to protect it and him or herself from discovery. Because fusion is transparent, the transporter is unrevealed and the data safeguarded against theft.
3.e. Quantum Memory Fusion can be used as a transport mechanism for educational material for children of all ages and grades, college students, adult learners, career and corporate training programs, vocational programs, vocabulary building, speech skills, presentation skills, foreign language skills, and all other educational programs and processes wherein some form of written or spoken word, or imagery is required or desired learning.
3.f. Quantum Memory Fusion can be used as a transport mechanism in the self-help industry whereby self-help trainers and motivational speakers may directly fuse recallable data onto the memory of the user. Such data could include, success habits, core success principles, or the personality constructs of successful role models.
3.g. Quantum Memory Fusion can be used as a transport mechanism for entertaining information such as story books for children, novels for more mature audiences, learning the rules of complex video games, or increasing knowledge with worldly facts about people, places, and things of interest.
3.h. Quantum Memory Fusion may be used as a transport mechanism for scripts and dialogues in film, cinema, drama, and stage.
3.i. Quantum Memory Fusion can be used as a transport mechanism for information that enhances skill and knowledge in sports, such as attitude towards the game and winning, increasing self-confidence, creating greater desire to train harder and longer, or predicating victory against one's opponent.
3.j. Quantum Memory Fusion can be used as a transport mechanism for political information such as political history or providing a method for global recall of a candidate's qualifications and messages.
3.k. Quantum Memory Fusion can be used as a transport mechanism for military information that enhances training of troops, efficiency with weapons, skills in combat, and lessons learned systems.
3.l. Quantum Memory Fusion can be used as a transport mechanism in law enforcement such as for training officers and agents, efficiency with weapons, rules and regulations, humanitarian training and people skills.
3.m. Quantum Memory Fusion can be used as a transport mechanism in the rehabilitation of youth in detention centers, incarcerated men and women, substance abuse recovery, and introduction to life skills.
3.n. Quantum Memory Fusion can be used as a transport mechanism for learning current manufacturing processes, technical processes, and specifications.
3.o. Quantum Memory Fusion can be used as a transport mechanism in commerce and industry to teach such skills as customer service, speaking and presentation, salesmanship, personal selling, relationship building, and competition skills.
3.p. Quantum Memory Fusion can be used as a transport mechanism for religious text and doctrines.
3.q. Quantum Memory Fusion can be used as an embryonic educational tool during pregnancy.
3.r. Quantum Memory Fusion can be used to deliver soothing information and instructions that assist in the training and pacification of animals.
3.s. Quantum Memory Fusion provides a means of effective advertising by fusing the advertiser's messages onto viewers, in a legal and ethical manner, thereby saving the advertiser a substantial sum on advertising expenses.
3.t. Quantum Memory Fusion provides an unsurpassed means for infomercial advertisers to fuse their messages onto the permanent recallable memory of viewers during a typical 30-minute infomercial, thereby reducing advertising expenses by up to 90% (ninety percent)
3.u. Quantum Memory Fusion can be used to create cognizant Neuromuscular Response (CNMR) by using video presentations with labeled movements such as in yoga, karate, boxing, fencing, combat, dancing, swimming, etc. These labeled movements are fused onto permanent recallable memory by name and image using QMF. Once engaged such as during competition or performance, the fused-labeled movements are returned as answers to action-based queries, thereby creating physical motion and tactics.
3.v. Quantum Memory Fusion is easily duplicable by a person skilled in the art and is suitable for mass manufacture and distribution by a plurality of manufacturers and distributors, and offers an extremely low production and raw materials cost, and has an unsurpassed availability rate for materials and equipment.
3.w. Quantum Memory Fusion offers an unappreciated advantage in that the data fused to permanent recallable memory appears to the test subjects as having always been available according to all test subjects. Test subjects found it unimaginable that it was not their “new found genius” and previously undiscovered “mental powers” that were responsible for the high recitation response and permanent memory recall. The test subjects desired all credit and that is exactly why Quantum Memory Fusion is perfect. QMF is transparent even to test subjects that are aware of its existence. This unappreciated advantage provides self-confidence and self-esteem that is an effective and subtle behavior modifier. I have noted that test subjects begun to seek new information more frequently from other sources and, from time to time, would recite the fused data as a show of mental superiority. Quantum Memory Fusion offers great rewards beyond those of the fusion process because children with learning disorders such as Attention Deficit Hyperactivity Disorder (ADHD) will learn, remember and seek new information without hesitation or prompting.
3.x. Quantum Memory Fusion has NOT been offered commercially.

### EVEN MORE OBJECTS AND ADVANTAGES

4.a. Quantum Memory Fusion can be deployed to any personal computer in software form. Software supporting QMF technology is easy to use because the user need only input the information to be fused and play-back this information for fusion to begin. No special skill is required.
4.b. Quantum Memory Fusion is safe for humans and animals. QMF does not alter, modify, or change the electrical properties of the brain. QMF uses only spoken words, images, or other sensory input.
4.c. Quantum Memory Fusion is easy to learn and deploy by any practitioner in computer science, behavioral science, neuroscience, psychiatry, psychology, and other psychotherapeutic fields or related arts.
4.d. Quantum Memory Fusion can be used on comatose patients in such a manner as to fuse the supportive phrases of family members and medical professionals onto the permanent memory of the patient. Even the final words of love from a family member would be appropriate to fuse onto memory of a terminally ill comatose patient. If the patient's auricular senses are intact, fusion will occur. Monroe, in U.S. Pat. No. 3,884,218 (1978) proved that the auricular senses continue to function even in deep sleep.

In this respect, before explaining at least one embodiment of the invention in detail, it is to be understood that the invention is not limited in its application to the details of construction and to the arrangements of the components set forth in the following description or illustrated in the drawings. The invention is capable of other embodiments, of being practiced, and carried out in various ways. In addition, it is to be understood that the phraseology and terminology employed herein are for the purpose of description and readability and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### DRAWING FIGURES

### Figures

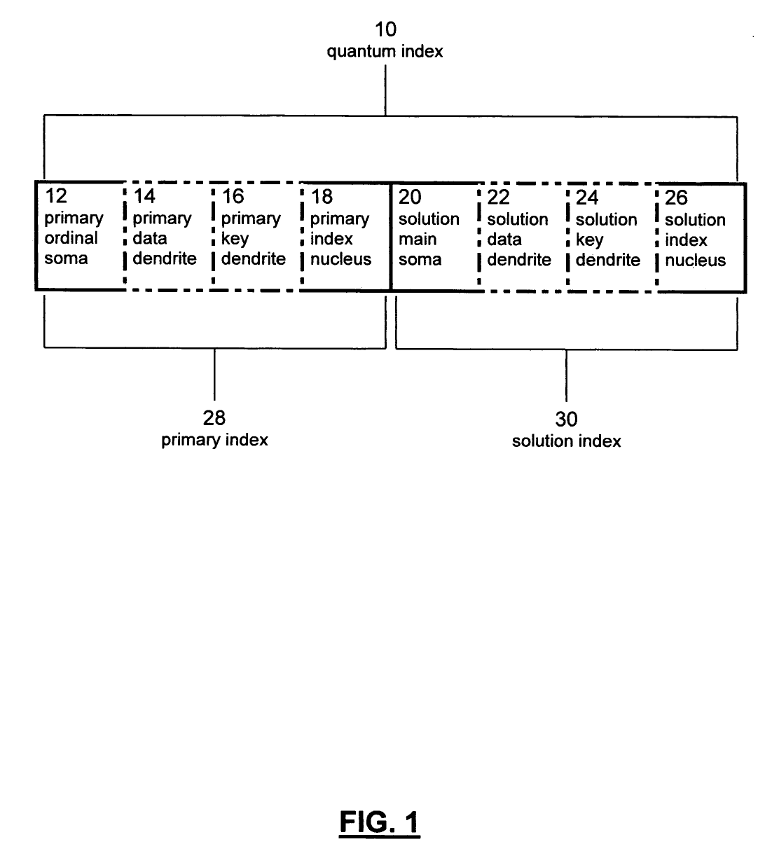

FIG. 1 is a segmented diagram of the of main Quantum Memory Fusion structure.

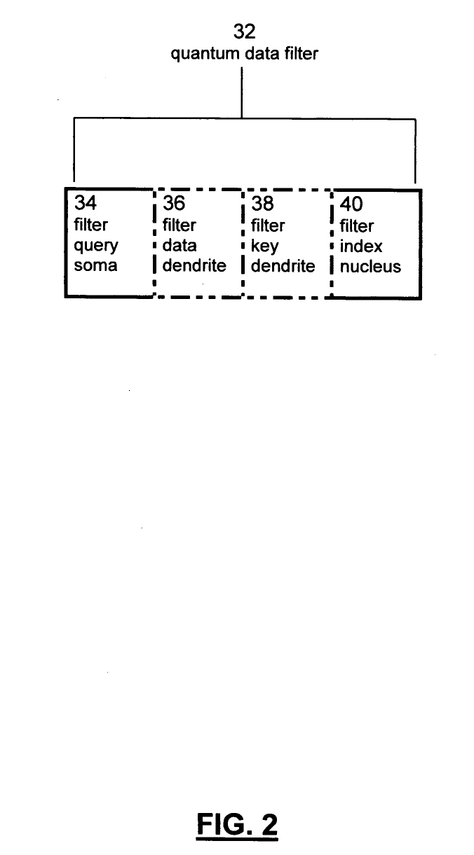

FIG. 2 is a segmented diagram of the Quantum Data Filter

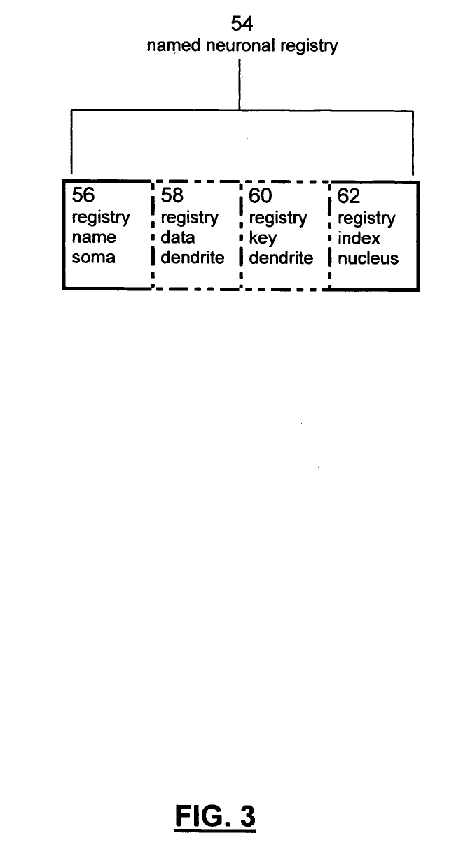

FIG. 3 is a segmented diagram of the Named Neuronal Registry.

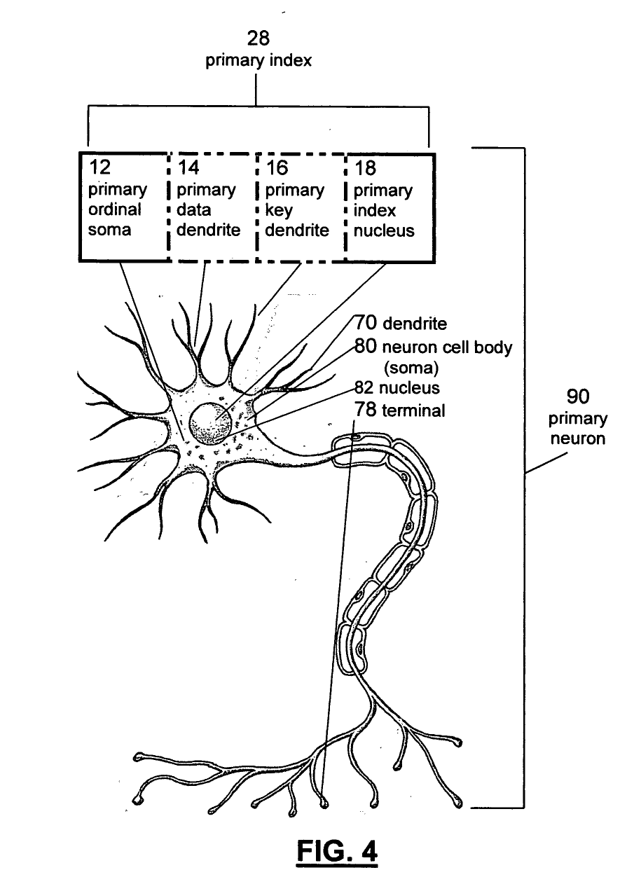

FIG. 4 is a segmented diagram of the Primary Index of the Primary Neuron and shows the relationship between segments in the Primary Index and components of the drawing of the Primary Neuron.

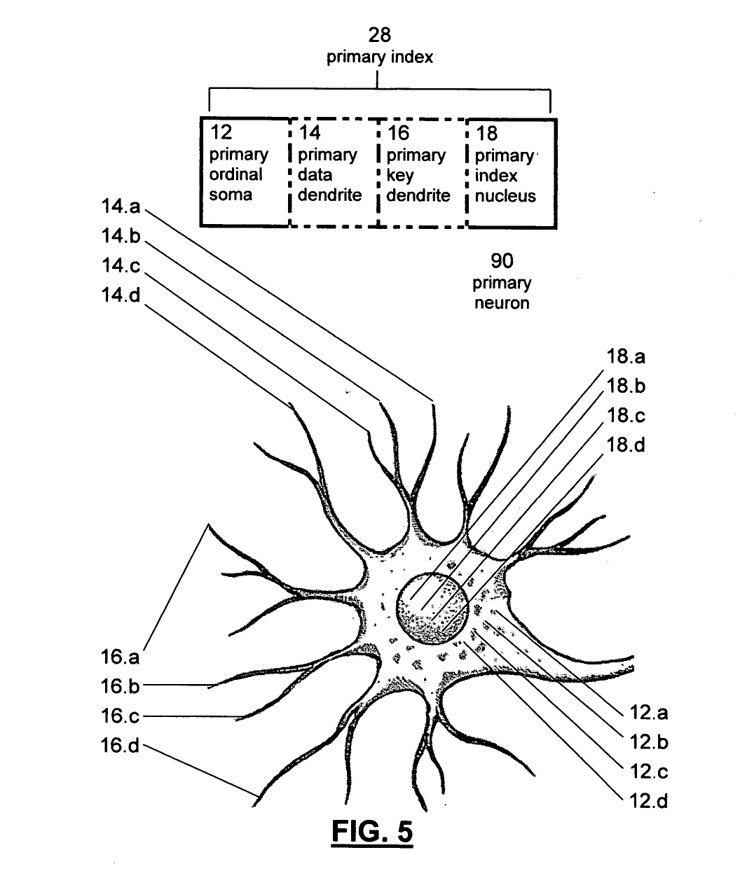

FIG. 5 is a segmented diagram of the Primary Index of the Primary Neuron and shows the relationship between segments in the Primary Index and components of the drawing of the Primary Neuron, in addition, the components of the Primary Index were replicated four times.

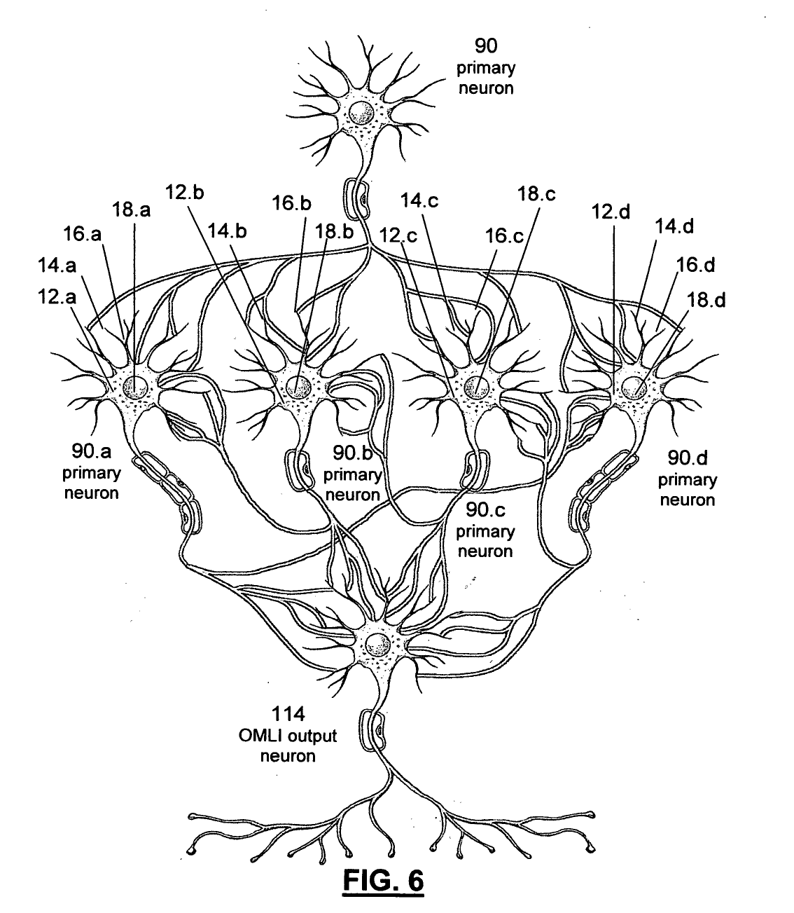

FIG. 6 is a drawing of the Primary Neuron with four additional neuron drawings attached. Each of the additional neurons represents new fused data.

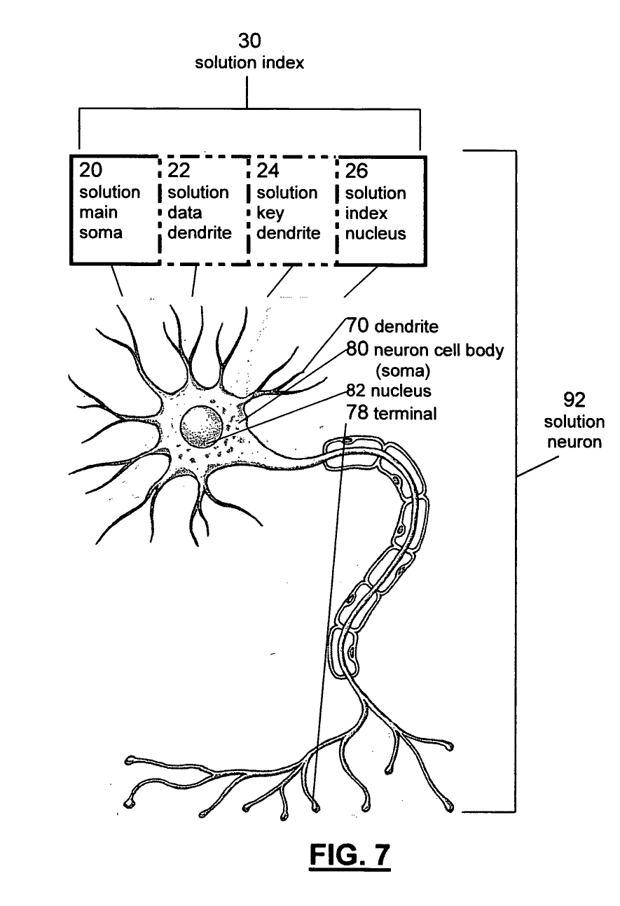

FIG. 7 is a segmented diagram of the Solution Index of the Solution Neuron and shows the relationship between segments in the Solution Index and components of the drawing of the Solution Neuron.

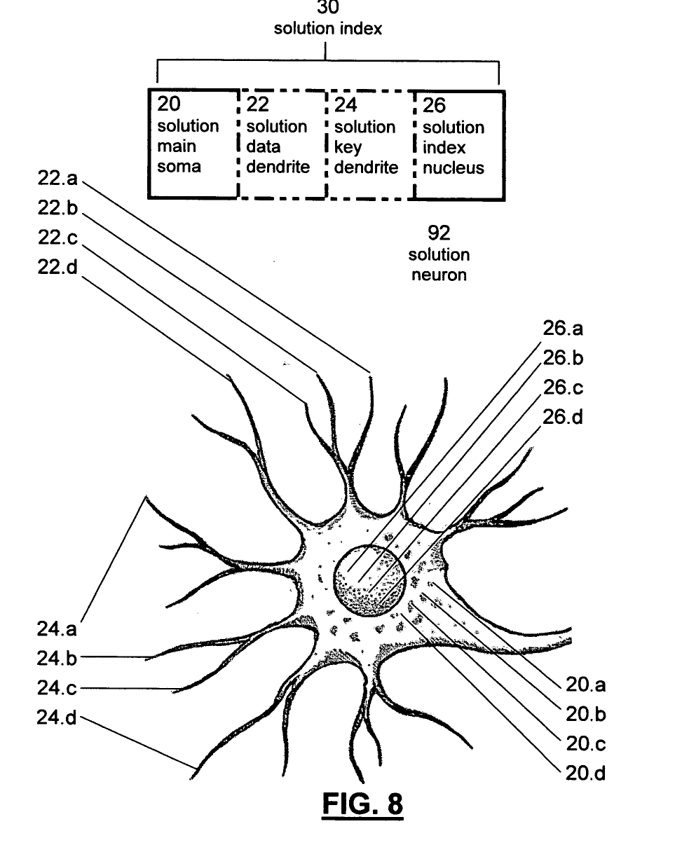

FIG. 8 is a segmented diagram of the Solution Index of the Solution Neuron and shows the relationship between segments in the Solution Index and components of the drawing of the Solution Neuron, in addition, the components of the Solution Index were replicated four times.

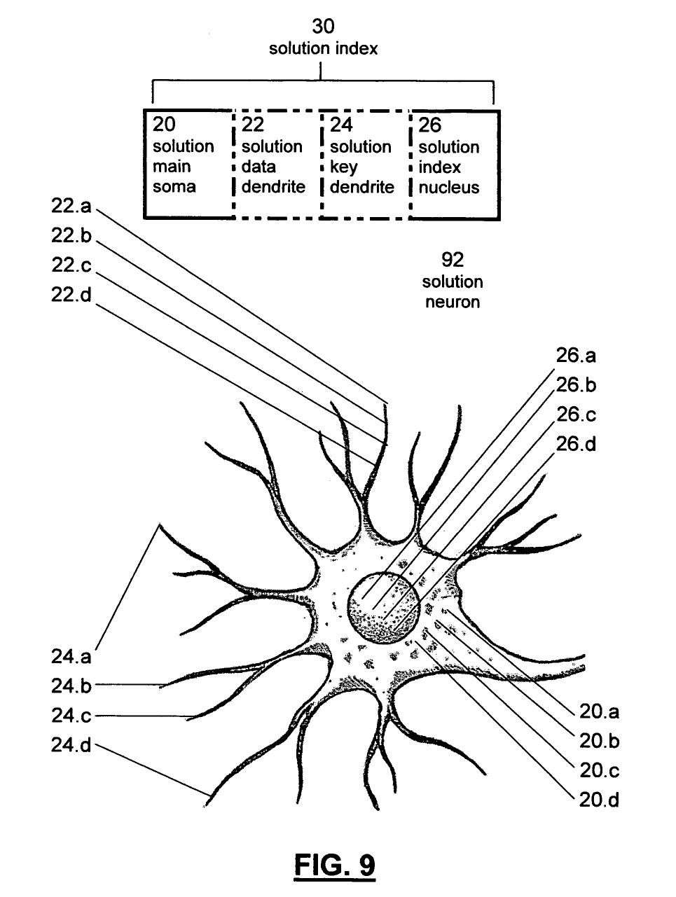

FIG. 9 is a segmented diagram of the Solution Index of the Solution Neuron and shows the relationship between segments in the Solution Index and components of the drawing of the Solution Neuron, in addition, the components of the Solution Index were replicated four times. The difference between FIG. 8 and FIG. 9 is that some components target different areas of the Solution Neuron.

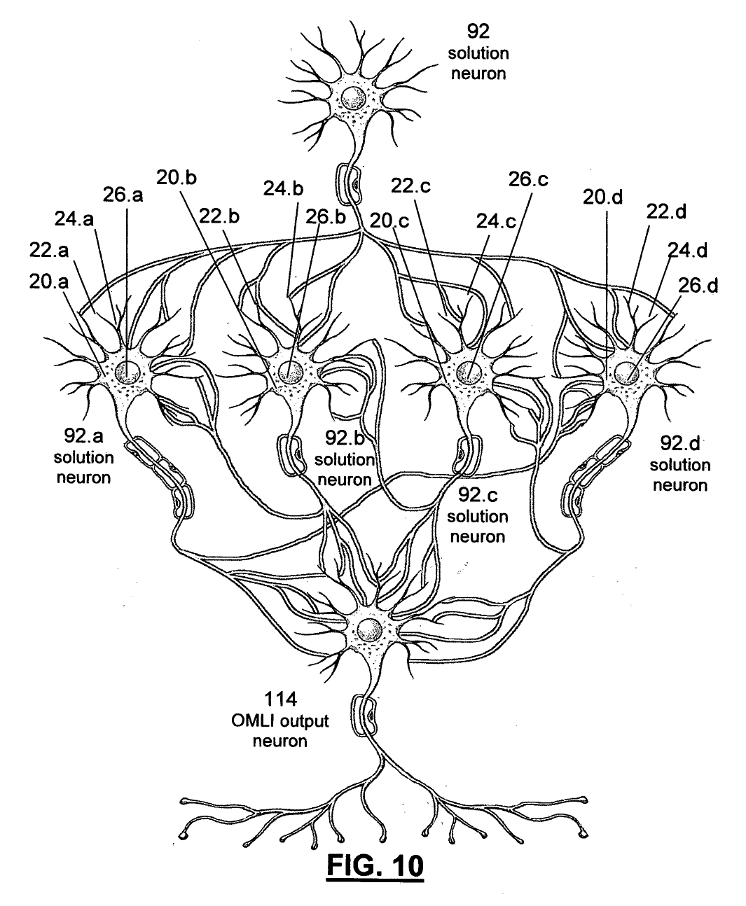

FIG. 10 is a drawing of the Solution Neuron with four additional neuron drawings attached. Each of the additional neurons represents new fused data.

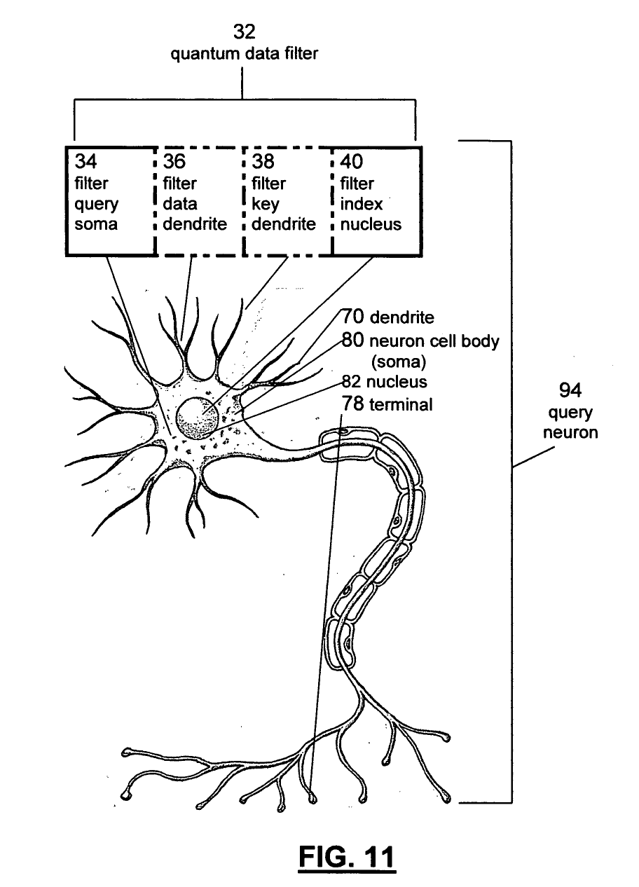

FIG. 11 is a segmented diagram of the Quantum Data Filter of the Query Neuron and shows the relationship between segments in the Quantum Data Filter and components of the drawing of the Query Neuron.

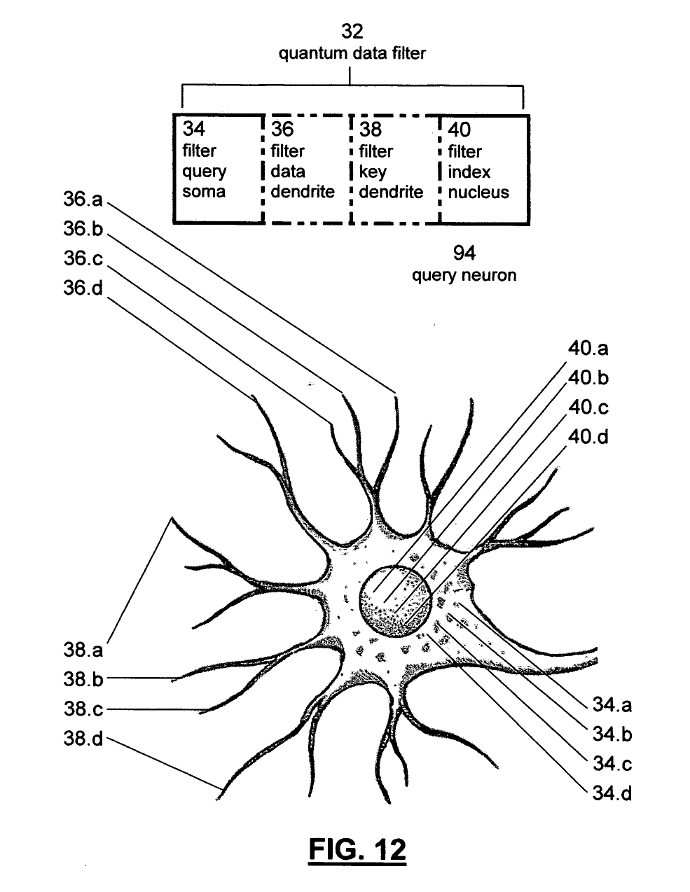

FIG. 12 is a segmented diagram of the Quantum Data Filter of the Query Neuron and shows the relationship between segments in the Quantum Data Filter and components of the drawing of the Query Neuron, in addition, the components of the Quantum Data Filter were replicated four times.

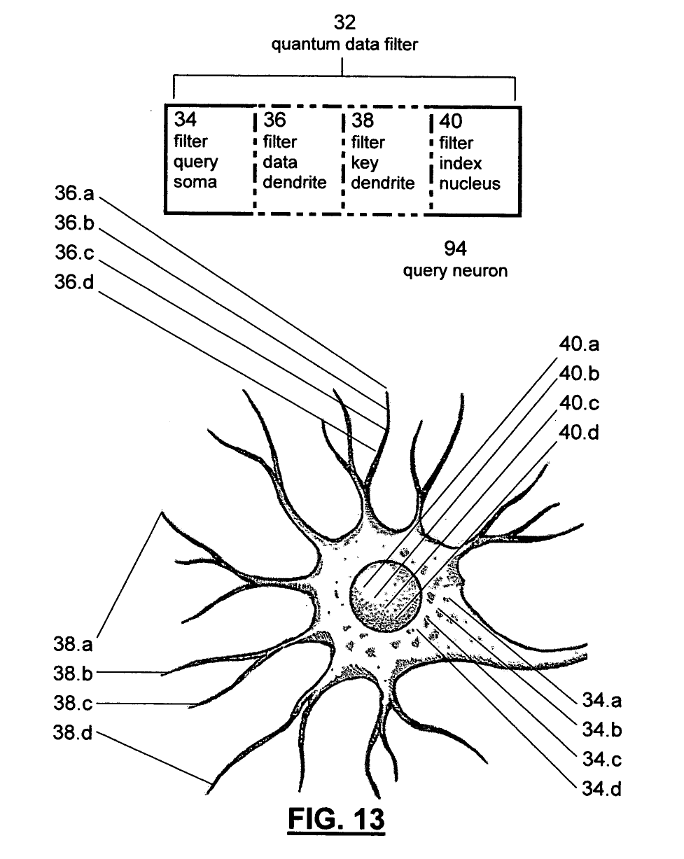

FIG. 13 is a segmented diagram of the Quantum Data Filter of the Query Neuron and shows the relationship between segments in the Quantum Data Filter and components of the drawing of the Query Neuron, in addition, the components of the Quantum Data Filter were replicated four times. The difference between FIG. 12 and FIG. 13 is that some components target different areas of the Query Neuron

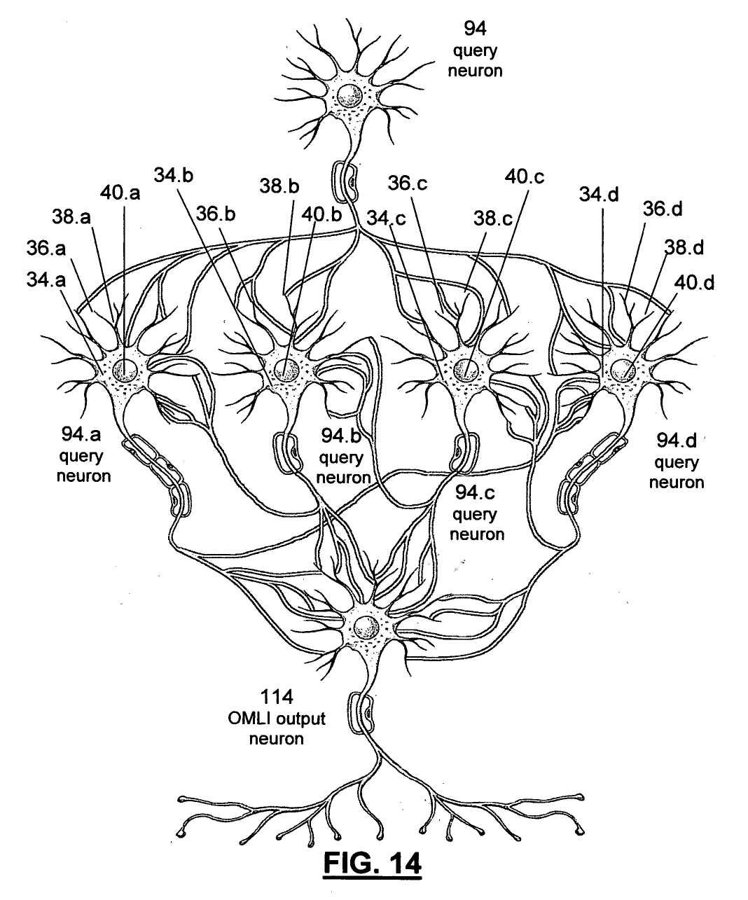

FIG. 14 is a drawing of the Query Neuron with four additional neuron drawings attached. Each of the additional neurons represents new fused data

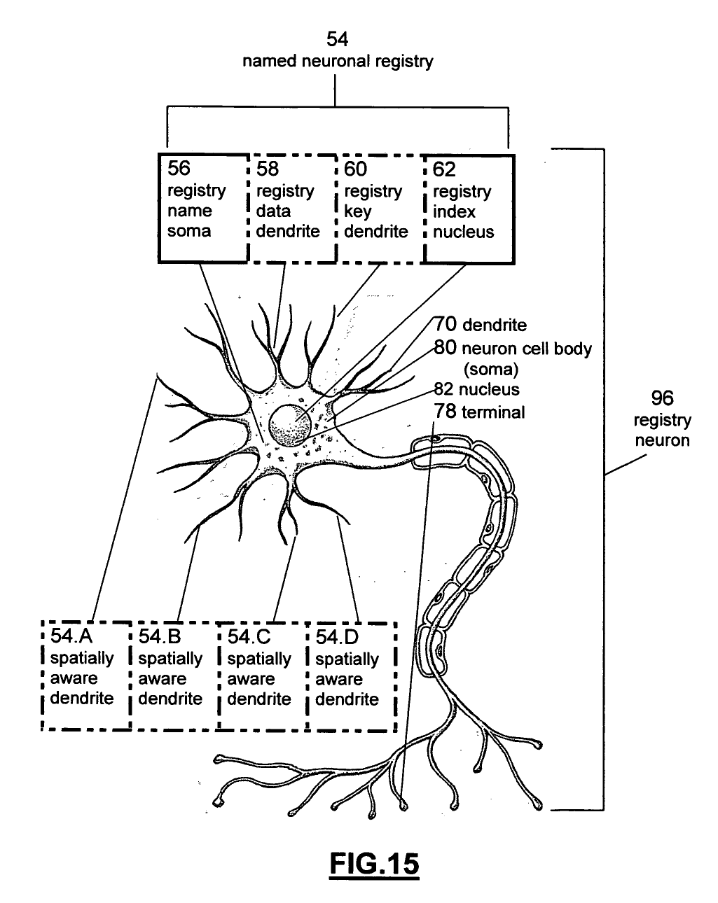

FIG. 15 is a segmented diagram of the Named Neuronal Registry of the Registry Neuron and shows the relationship between segments in the Named Neuronal Registry and components of the drawing of the Registry Neuron.

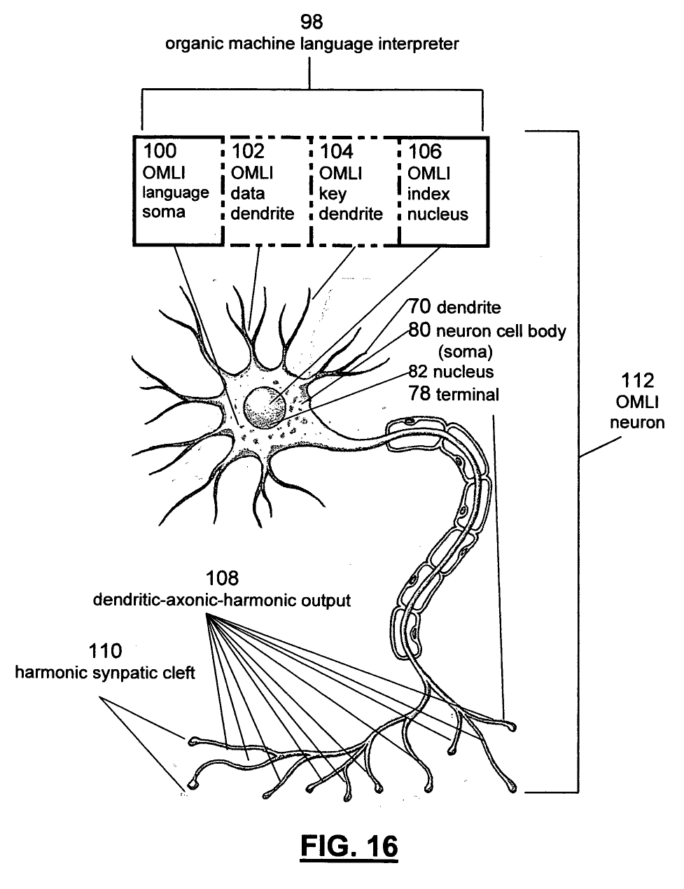

FIG. 16 is a segmented diagram of the organic Machine Language Interpreter of the OMLI Neuron and shows the relationship between segments in the organic Machine Language Interpreter and components of the drawing of the OMLI Neuron.

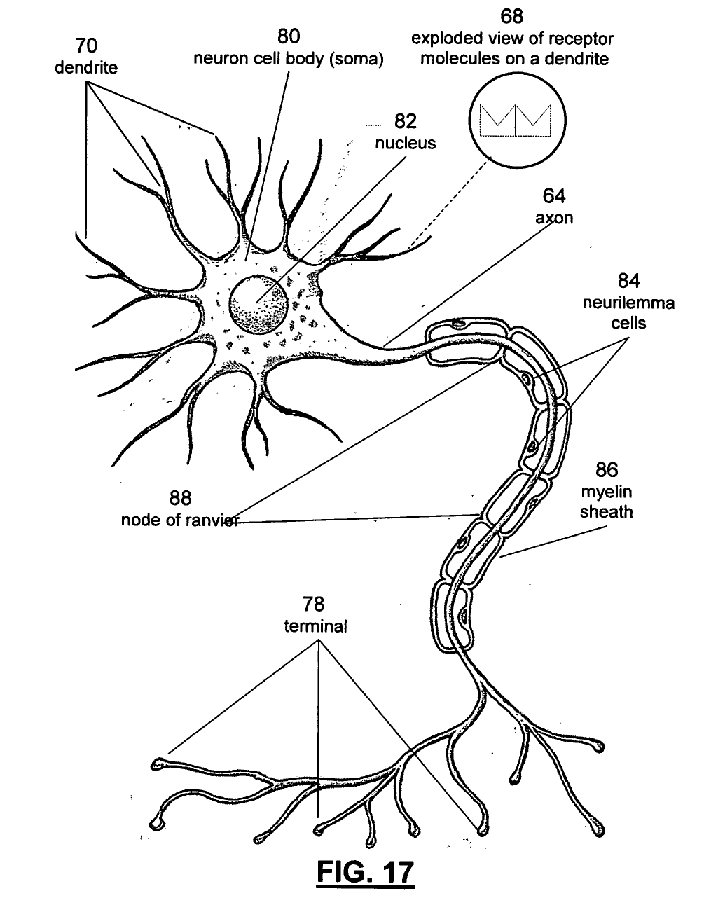

FIG. 17 is a drawing of a neuron with named components.

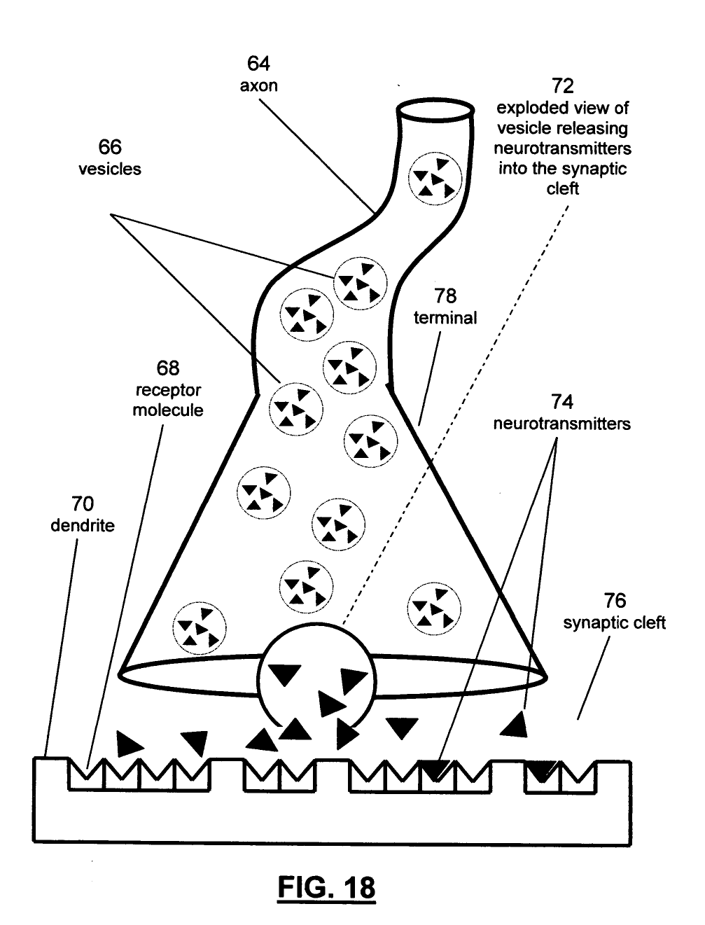

FIG. 18 is a drawing of a presynaptic terminal releasing neurotransmitters into the synaptic cleft of a dendrite.

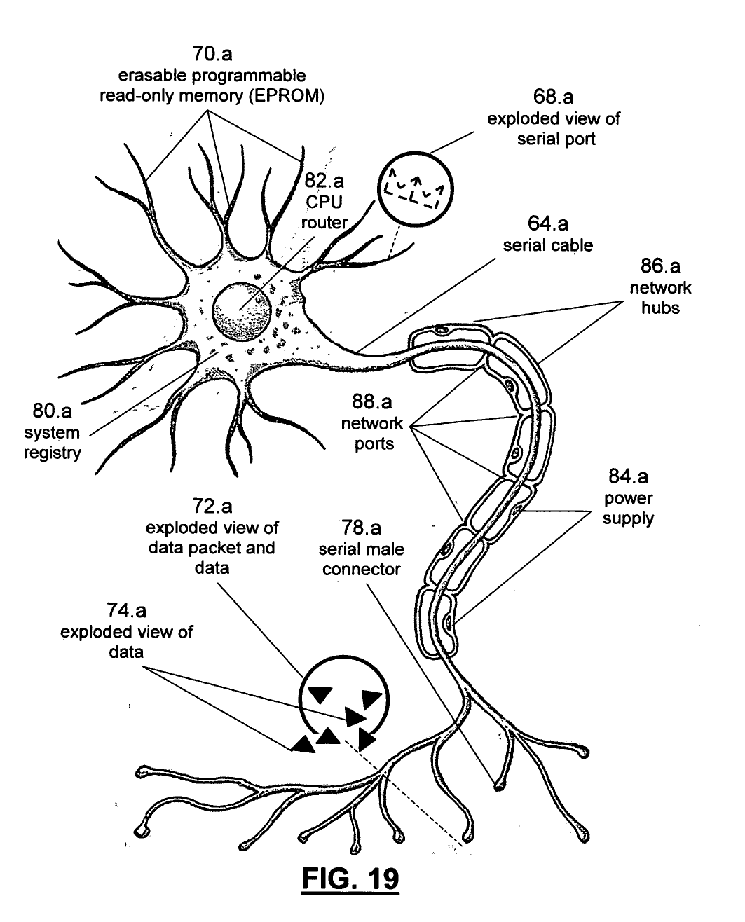

FIG. 19 is a drawing of a neuron on which the organic component names were replaced with comparable inorganic machine part names.

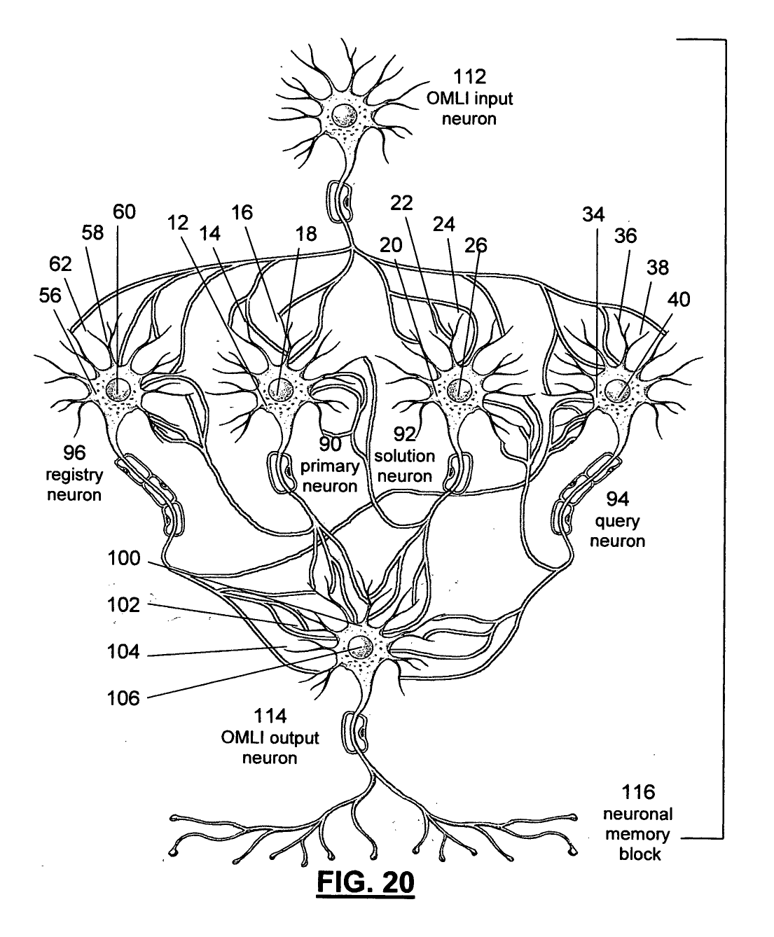

FIG. 20 is a drawing of a complete Neuronal Memory Block

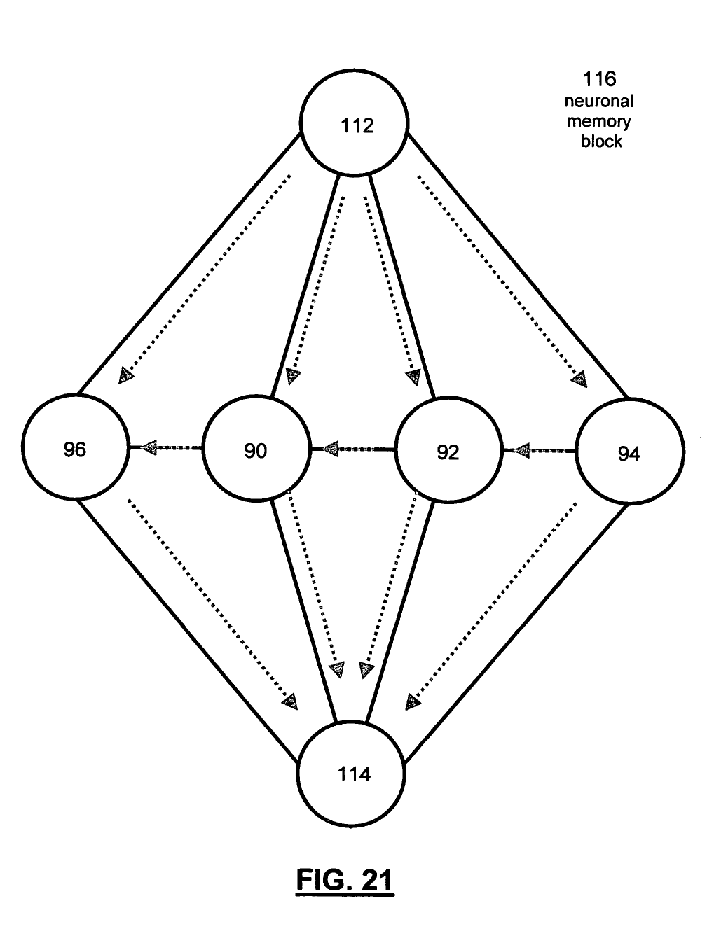

FIG. 21 is a drawing of the symbolic diamond shape of a Neuronal Memory Block.

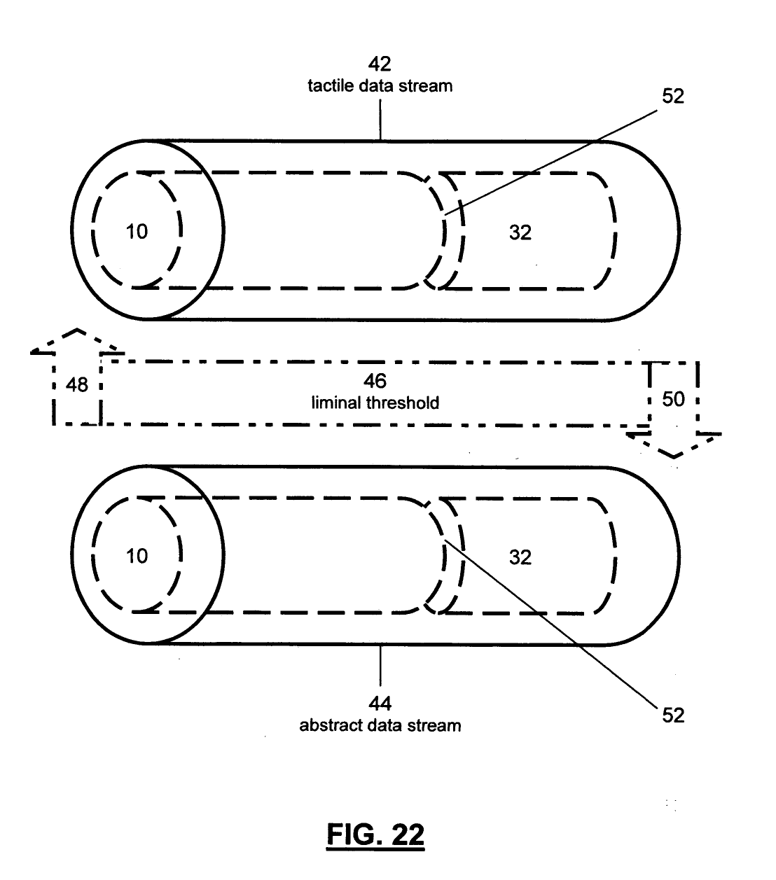

FIG. 22 is a drawing of an audio data stream containing the primary index and solution index only, with output at and below the threshold of human hearing.

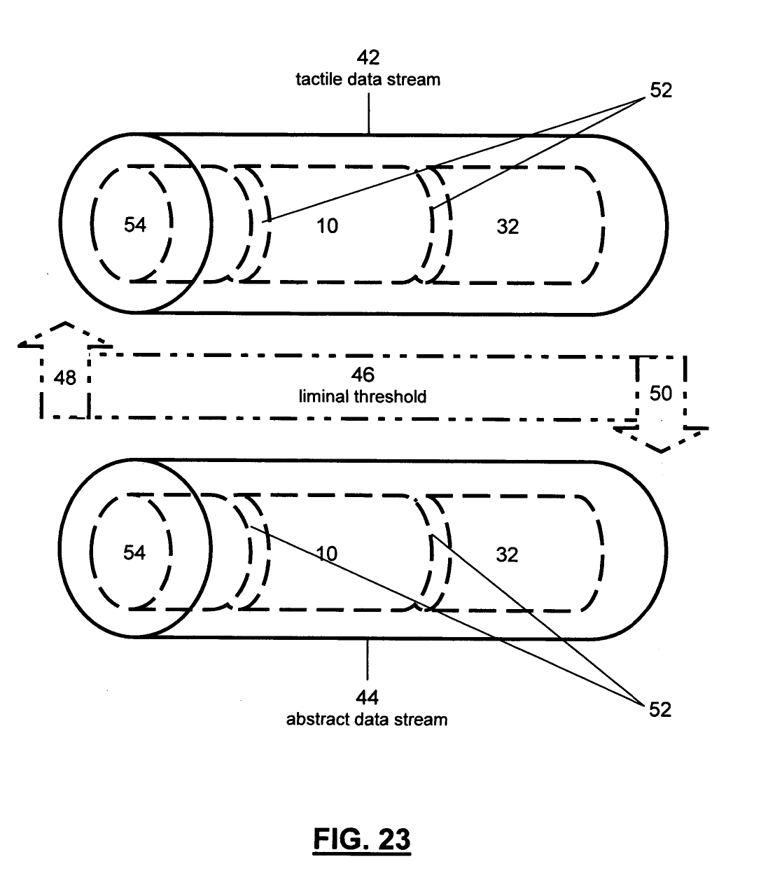

FIG. 23 is a drawing of an audio data stream containing the named neuronal registry, primary index and solution index only, with output at and below the threshold of human hearing.

FIG. 23 is a drawing of an audio data stream containing the named neuronal registry, primary index and solution index only, with output at and below the threshold of human hearing.

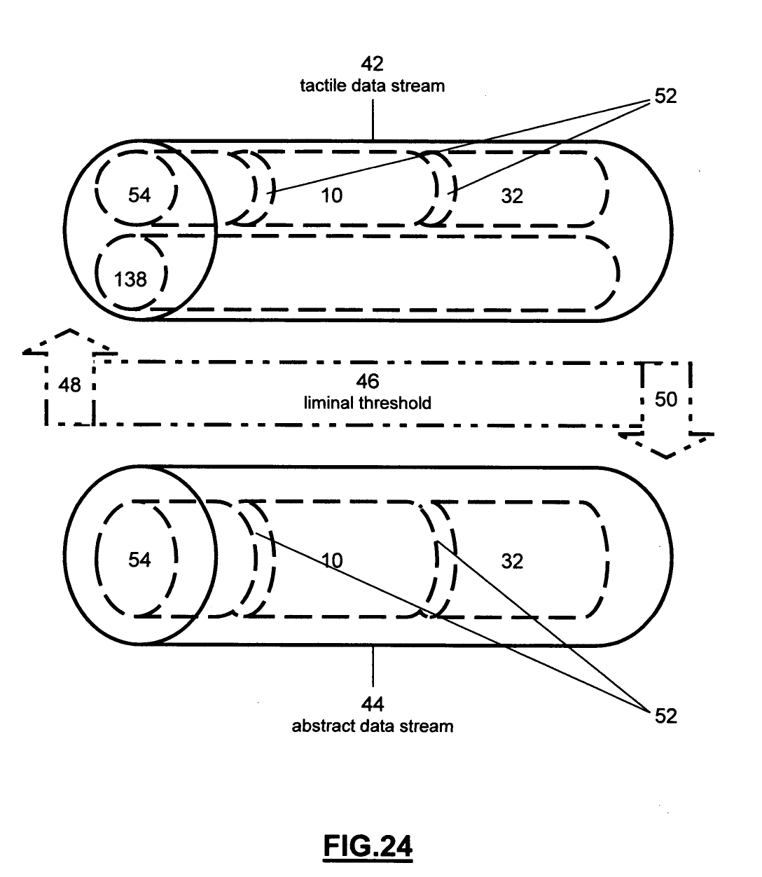

FIG. 24 is a drawing of a liminal audio and video data stream containing the Named Neuronal Registry, Primary Index and Solution Index only, with output at and below the threshold of human hearing. No video data is output below the limen.

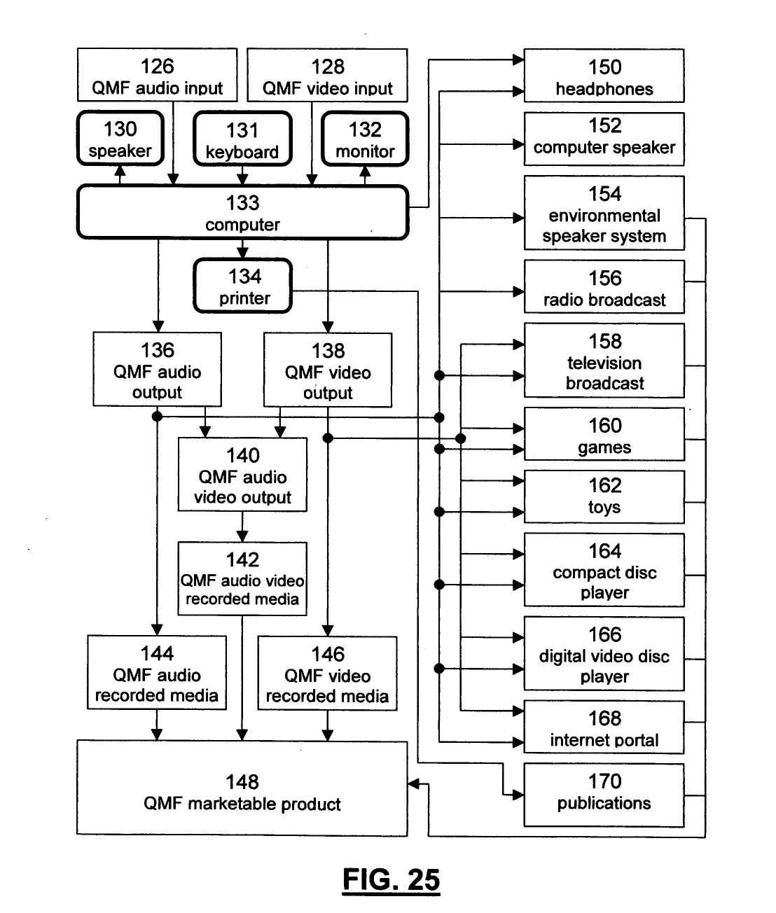

FIG. 25 is a flow chart illustrating the recording process, output methods, and distribution mediums for Quantum Memory Fusion.

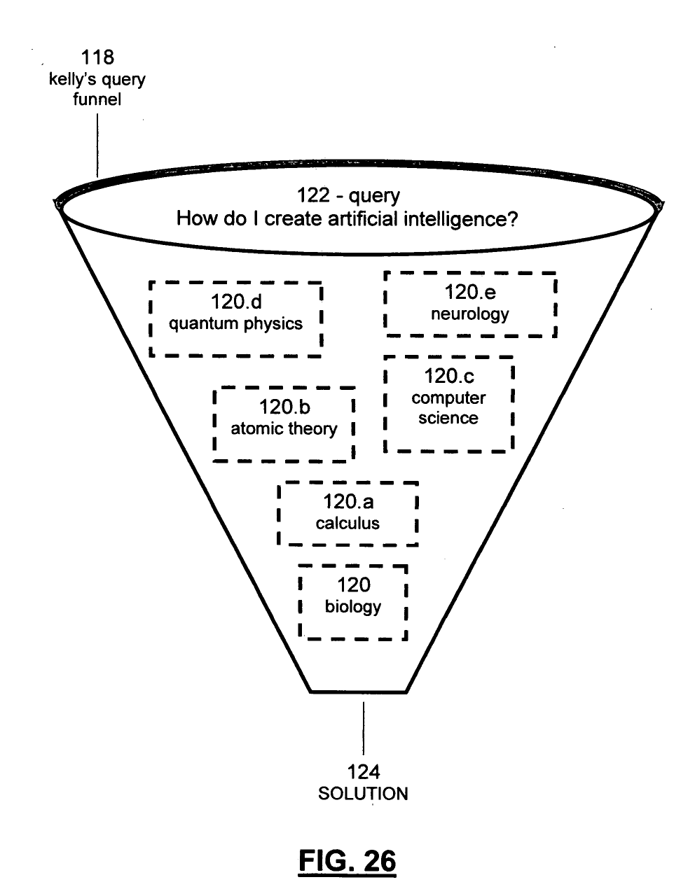

FIG. 26 is a drawing of Kelly's Query Funnel as used to solve irresolvable questions.

### TABLE OF REFERENCE NUMERALS

TABLE 3-1
Reference Numerals

| Numeral | Description |
|---|---|
| 10 | quantum index |
| 12 | primary ordinal soma |
| 12.a | primary ordinal soma |
| 12.b | primary ordinal soma |
| 12.c | primary ordinal soma |
| 12.d | primary ordinal soma |
| 14 | primary data dendrite |
| 14.a | primary data dendrite |
| 14.b | primary data dendrite |
| 14.c | primary data dendrite |
| 14.d | primary data dendrite |
| 16 | primary key dendrite |
| 16.a | primary key dendrite |
| 16.b | primary key dendrite |
| 16.c | primary key dendrite |
| 16.d | primary key dendrite |
| 18 | primary index nucleus |
| 18.a | primary index nucleus |
| 18.b | primary index nucleus |
| 18.c | primary index nucleus |
| 18.d | primary index nucleus |
| 20 | solution main soma |
| 20.a | solution main soma |
| 20.b | solution main soma |
| 20.c | solution main soma |
| 20.d | solution main soma |
| 22 | solution data dendrite |
| 22.a | solution data dendrite |
| 22.b | solution data dendrite |
| 22.c | solution data dendrite |
| 22.d | solution data dendrite |
| 24 | solution key dendrite |
| 24.a | solution key dendrite |
| 24.b | solution key dendrite |
| 24.c | solution key dendrite |
| 24.d | solution key dendrite |
| 26 | solution index nucleus |
| 26.a | solution index nucleus |
| 26.b | solution index nucleus |
| 26.c | solution index nucleus |
| 26.d | solution index nucleus |
| 28 | primary index |
| 30 | solution index |
| 32 | quantum data filter |
| 34 | filter query soma |
| 34.a | filter query soma |
| 34.b | filter query soma |
| 34.c | filter query soma |
| 34.d | filter query soma |
| 36 | filter data dendrite |
| 36.a | filter data dendrite |
| 36.b | filter data dendrite |
| 36.c | filter data dendrite |
| 36.d | filter data dendrite |
| 38 | filter key dendrite |
| 38.a | filter key dendrite |
| 38.b | filter key dendrite |
| 38.c | filter key dendrite |
| 38.d | filter key dendrite |
| 40 | filter index nucleus |
| 40.a | filter index nucleus |
| 40.b | filter index nucleus |
| 40.c | filter index nucleus |
| 40.d | filter index nucleus |
| 42 | tactile data stream |
| 44 | abstract data stream |
| 46 | liminal threshold |
| 48 | ascending threshold |
| 50 | descending threshold |
| 52 | natural neuronal buffer |
| 54 | named neuronal registry |
| 54.A | spatially aware dendrite |
| 54.B | spatially aware dendrite |
| 54.C | spatially aware dendrite |
| 54.D | spatially aware dendrite |
| 56 | registry name soma |
| 58 | registry data dendrite |
| 60 | registry key dendrite |
| 62 | registry index nucleus |
| 64 | axon |
| 64.a | serial cable |
| 66 | vesicle |
| 68 | exploded view of receptor molecules on a dendrite |
| 70 | dendrite |
| 70.a | erasable programmable read-only memory (EPROM) |
| 72 | exploded view of vesicle releasing neurotransmitters |
| | into the synaptic cleft |
| 72.a | exploded view of data packet and data |
| 74 | neurotransmitters |
| 74.a | exploded view of data |
| 76 | synaptic cleft |
| 78 | terminal |
| 78.a | serial male connector |
| 80 | neuron cell body (soma) |
| 80.a | system registry |
| 82 | Nucleus |
| 82.a | CPU router |
| 84 | neurilemma cells |
| 84.a | power supply |
| 86 | myelin sheath |
| 86.a | network hubs |
| 88 | node of ranvier |
| 88.a | network ports |
| 90 | primary neuron |
| 90.a | primary neuron |
| 90.b | primary neuron |
| 90.c | primary neuron |
| 90.d | primary neuron |
| 92 | solution neuron |
| 92.a | solution neuron |
| 92.b | solution neuron |
| 92.c | solution neuron |
| 92.d | solution neuron |
| 94 | query neuron |
| 94.a | query neuron |
| 94.b | query neuron |
| 94.c | query neuron |
| 94.d | query neuron |
| 96 | registry neuron |
| 98 | OMLI neuron |
| 100 | OMLI language soma |
| 102 | OMLI data dendrite |
| 104 | OMLI key dendrite |
| 106 | OMLI index nucleus |
| 108 | dendritic-axonic-harmonic-output |
| 110 | harmonic synaptic cleft |
| 112 | OMLI input neuron |
| 114 | OMLI output neuron |
| 116 | neuronal memory block |
| 118 | kelly's query funnel |
| 120 | input topic-biology |
| 120.a | input topic-calculus |
| 120.b | input topic-atomic theory |
| 120.c | input topic-computer science |
| 120.d | input topic-quantum physics |
| 120.e | input topic-neurology |
| 122 | query “How do I create artificial intelligence?” |
| 124 | solution |
| 126 | QMF audio input |
| 128 | QMF video input |
| 130 | speaker |
| 131 | keyboard |
| 132 | monitor |
| 133 | computer |
| 134 | printer |
| 136 | QMF audio output |
| 138 | QMF video output |
| 140 | QMF audio video output |
| 142 | QMF audio video recorded media |
| 144 | QMF audio recorded media |
| 146 | QMF video recorded media |
| 148 | QMF marketable product |
| 150 | headphones |
| 152 | computer speaker |
| 154 | environmental speaker system |
| 156 | radio broadcast |
| 158 | television broadcast |
| 160 | games |
| 162 | toys |
| 164 | compact disc player |
| 166 | digital video disc player |
| 168 | internet portal |
| 170 | publications |

### DETAILED DESCRIPTION

FIG. 1 shows a block diagram of one embodiment of quantum index 10 of the Quantum Memory Fusion method. At the heart of 10 is primary ordinal soma 12, primary data dendrite 14, primary key dendrite 16, primary index nucleus 18, solution main soma 20, solution data dendrite 22, solution key dendrite 24, and solution index nucleus 26. Blocks 12,14,16,18,20,22,24 and 26 may contain transmittable outbound data strings and or video sequences or values. Quantum Index 10 is further separated into two indexes, primary index 28 and solution index 30.

FIG. 2 shows a block diagram of a preferred embodiment of quantum data filter 32. At the heart of 32 is filter query soma 34, filter data dendrite 36, filter key dendrite 38, and filter index nucleus 40. Blocks 34,36,38, and 40 may contain transmittable outbound data strings or video sequences or values.

FIG. 3 shows a block diagram of an embodiment of named neuronal registry 54. At the heart of 54 is registry name soma 56, registry data dendrite 58, registry key dendrite 60, and registry index nucleus 62. Blocks 56,58,60, and 62 may contain transmittable outbound data strings or video sequences, values, or environmental variables supplied by the brain, such as time, date, body temperature, emotional state, age, location, etc.

FIG. 4 is a one embodiment of primary neuron 90 and primary index 28. From primary index 28, primary ordinal soma 12 theoretically transmits a value to neuron cell body (soma) 80. Primary data dendrite 14 theoretically transmits data to a dendrite 70. Primary key dendrite 16 theoretically transmits data to a different dendrite 70. Primary index nucleus 18 theoretically transmits data to nucleus 82.

FIG. 5 is another view of primary neuron 90 and primary index 28. This drawing excludes axon 64 from view. From this perspective, primary ordinal somas 12-12.d each theoretically transmit a same value to neuron cell body (soma) 80. Then, primary data dendrites 14-14.d each theoretically transmit unique data to a corresponding dendrite 70. Now, primary key dendrites 16-16.d each theoretically transmit unique data to a corresponding dendrite 70. Finally, primary index nucleus 18-18.d each theoretically transmit a same value to nucleus 82.

FIG. 6 is a drawing of primary neuron 90 with primary neurons 90.a, 90.b, 90.c, and 90.d each attached to a corresponding group of axon 64 on primary neuron 90. In this view, 90-90.d each receive unique neurotransmitters 74 from 12-12.d, 14-14.d, 16-16.d, and 18-18.d. This theoretical figure represents a unique value in each primary ordinal soma 12-12.d and unique data in each primary index nucleus 18-18.d as transmitted by primary neuron 90. Primary neuron 90 stores a single unique value in primary ordinal soma 12 and a single unique data string or video sequence in primary index nucleus 18. For each additional unique primary ordinal soma value and primary index nucleus data string or video sequence, a new neuron is called to primary neuron 90 to store the new value or data string or video sequence. The new neuron creates a synaptic cleft 76 in a pseudo-attached mode between terminal 78 on primary neuron 90 and at a group of dendrite 70 on a new neuron. The new neuron makes a pseudo-attachment to OMLI output neuron 114 via the new neuron terminal 78 and the OMLI output neuron 114 dendrite 70. The pseudo-attachment between terminal 78 and dendrite 70 can be a single connection to a plurality of connections between any two or more neurons. Clockwise from left to right, four neurons are present. They are, 90.a, 90.b, 90.c, and 90.d. Each of the four neurons connects to the dendrite 70 of OMLI output neuron via their respective terminal 78. However, neurons output and read data counter clockwise, that is from right to left. Now, terminal 78 of 90.d connects to dendrite 70 of 90.c. Then, terminal 78 of 90.c connects to dendrite 70 of 90.b. Next, terminal 78 of 90.b connects to dendrite 70 of 90.a. Finally, terminal 78 of 90.a connects back to dendrite 70 of 90.d in a three-dimensional diamond shape.

FIG. 7 is a one embodiment of solution neuron 92 and solution index 30. From solution index 30, solution main soma 20 theoretically transmits a value to neuron cell body (soma) 80. Solution data dendrite 22 theoretically transmits data to a dendrite 70. Solution key dendrite 24 theoretically transmits data to a different dendrite 70. Solution index nucleus 28 theoretically transmits data to nucleus 82.

FIG. 8 is another view of solution neuron 92 and solution index 30. This drawing excludes axon 64 from view. From this perspective, solution main soma 20-20.d each theoretically transmit a same value to neuron cell body (soma) 80. Then, solution data dendrites 22-22.d each theoretically transmit unique data to a corresponding dendrite 70. Now, solution key dendrites 24-24.d each theoretically transmit unique data to a corresponding dendrite 70. Finally, solution index nucleus 26-26.d each theoretically transmit a same value to nucleus 82.

FIG. 9 is yet another view of solution neuron 92. This view differs from FIG. 8 in that solution data dendrites 22-22.d each theoretically transmit the same data to a single dendrite 70.

FIG. 10 is a drawing of solution neuron 92 with solution neurons 92.a, 92.b, 92.c, and 92.d each attached to a corresponding group of axon 64 on solution neuron 92. In this view, 92-92.d each receive unique neurotransmitters 74 from 20-20.d, 22-22.d, 24-24.d, and 26-26.d. This theoretical figure represents a unique value in each solution main soma 20-20.d and unique data in each solution index nucleus 26-26.d as transmitted by solution neuron 92. Solution neuron 92 stores a single unique value in solution main soma 20 and a single unique data string or video sequence in solution index nucleus 26. For each additional unique solution main soma value and solution index nucleus data string or video sequence, a new neuron is called to solution neuron 92 to store the new value or data string or video sequence. The new neuron creates a synaptic cleft 76 in a pseudo-attached mode between terminal 78 on solution neuron 92 and at a group of dendrite 70 on a new neuron. The new neuron makes a pseudo-attachment to OMLI output neuron 114 via the new neuron terminal 78 and the OMLI output neuron 114 dendrite 70. The pseudo-attachment between terminal 78 and dendrite 70 can be a single connection to a plurality of connections between any two or more neurons. Clockwise from left to right, four neurons are present. They are, 92.a, 92.b, 92.c, and 92.d. Each of the four neurons connects to the dendrite 70 of OMLI output neuron 114 via their respective terminal 78. However, neurons output and read data counter clockwise, that is from right to left. NOW, terminal 78 of 92.d connects to dendrite 70 of 92.c. Then, terminal 78 of 92.c connects to dendrite 70 of 92.b. Next, terminal 78 of 92.b connects to dendrite 70 of 92.a. Finally, terminal 78 of 92.a connects back to dendrite 70 of 92.d in a three-dimensional diamond shape.

FIG. 11 is a one embodiment of query neuron 94 and quantum data filter 32. From quantum data filter 32, filter query soma 34 theoretically transmits a value to neuron cell body (soma) 80. Filter data dendrite 36 theoretically transmits data to a dendrite 70. Filter key dendrite 38 theoretically transmits data to a different dendrite 70. Filter index nucleus 40 theoretically transmits data to nucleus 82.

FIG. 12 is another view of query neuron 94 and quantum data filter 32. This drawing excludes axon 64 from view. From this perspective, filter query somas 34-34.d each theoretically transmit a save value to neuron cell body (soma) 80. Then, filter data dendrites 36-36.d each theoretically transmit unique data to five separate dendrites 70. Now, filter key dendrites 38-38.d each theoretically transmit unique data to five separate dendrites 70. Finally, filter index nucleus 40-40.d each theoretically transmit a same value to nucleus 82.

FIG. 13 is still another view of query neuron 94. This view differs from FIG. 12 in that filter data dendrites 36-36.d each theoretically transmit the same data to a single dendrite 70.

FIG. 14 is a drawing of query neuron 94 with query neurons 94.a, 94.b, 94.c, and 94.d each attached to a corresponding group of axon 64 on query neuron 94. In this view, 94-94.d each receive unique neurotransmitters 74 from 34-34.d, 36-36.d, 38-38.d, and 40-40.d. This theoretical figure represents a unique value in each filter query soma 34-34.d and unique data in each filter index nucleus 40-40.d as transmitted by query neuron 94. Query neuron 94 stores a single unique value in filter query soma 34 and a single unique data string or video sequence in filter index nucleus 40. For each additional unique filter query soma value and filter index nucleus data string or video sequence, a new neuron is called to query neuron 94 to store the new value or data string or video sequence. The new neuron creates a synaptic cleft 76 in a pseudo-attached mode between terminal 78 on query neuron 94 and at a group of dendrite 70 on a new neuron. The new neuron makes a pseudo-attachment to OMLI output neuron 114 via the new neuron terminal 78 and the OMLI output neuron 114 dendrite 70. The pseudo-attachment between terminal 78 and dendrite 70 can be a single connection to a plurality of connections between any two or more neurons. Clockwise from left to right, four neurons are present. They are, 94.a, 94.b, 94.c, and 94.d. Each of the four neurons connects to the dendrite 70 of OMLI output neuron 114 via their respective terminal 78. However, neurons output and read data counter clockwise, that is from right to left. Now, terminal 78 of 94.d connects to dendrite 70 of 94.c. Then, terminal 78 of 94.c connects to dendrite 70 of 94.b. Next, terminal 78 of 94.b connects to dendrite 70 of 94.a. Finally, terminal 78 of 94.a connects back to dendrite 70 of 94.d in a three-dimensional diamond shape.

FIG. 15 is a one embodiment of registry neuron 96 and named neuronal registry 54. From named neuronal registry 54, registry name soma 56 theoretically transmits a value to neuron cell body (soma) 80. Registry data dendrite 58 theoretically transmits data to a dendrite 70. Registry key dendrite 60 theoretically transmits data to a different dendrite 70. Registry index nucleus 62 theoretically transmits data to nucleus 82. Spatially aware dendrite 54.A, 54.B, 54.C, and 54.D each theoretically transmits environmental variables supplied by the brain, such as time, date, body temperature, emotional state, age, location, etc., to a separate dendrite on registry neuron 96.

FIG. 16 is one embodiment of OMLI neuron 112 and organic machine language interpreter 98. From organic machine language interpreter 98, OMLI language soma 100 theoretically transmits a value to neuron cell body (soma) 80. OMLI data dendrite 102 theoretically transmits data to a dendrite 70. OMLI key dendrite 104 theoretically transmits data to a different dendrite 70. OMLI index nucleus 106 theoretically transmits data to nucleus 82 on OMLI neuron 112. Next, dendritic-axonic-harmonic output 108 theoretically transmits harmonic representations of organic machine language data across harmonic synaptic cleft 110. OMLI neuron 112 is unique among the neurons in neuronal memory block 116 because it receives its input in organic machine language from other neurons in a neuronal memory block 116 and then converts this input to the native language in which the original input was stored. Other neurons in a neuronal memory block 116 receive and transmit data in organic machine language only. OMLI neuron 112 maintains the status of languages available to a neuronal memory block 116.

FIG. 17 is a drawing of a neuron. This exemplar in theorem depicts a dendrite 70 that stores data, neuron cell body (soma) 80 that serves as a named pointer to data stored in a dendrite 70, and nucleus 82 that acts as a named switching mechanism or router to redirect data found by 80 and stored in 70 to a terminal 78 at the end of a axon 64. Nucleus 82 further tags the data it transmits to axon 64 that transports data away from nucleus 82. Node of Ranvier accepts connections at the node via a terminal 78 of another neuron. Neurilemma cells 84 power the myelin sheath 86 which directs the tagged output from nucleus 82 to the appropriate terminal 78 via axon 64 which serves as conduit for vesicles 66 to a dendrite 70 of another neuron, and finally releases neurotransmitters 74 into the synaptic cleft 76. Then, neurotransmitters 74 attach to receptor molecule 66 on a dendrite 70. This process is the data transfer.

FIG. 18 is self-explanatory.

FIG. 19 is self-explanatory.

FIG. 20 is a drawing of a neuronal memory block 116. This theoretical diamond configuration affords an OMLI input neuron 112 at the apex of the diamond that connects to the dendrite 70 of 96,90,92, and 94 via axon 64 and terminal 78 on 112. OMLI output neuron 114 sits at the base of the diamond. Clockwise from left to right, four neurons are present. They are, registry neuron 96, primary neuron 90, solution neuron 92, and query neuron 94. Each of the four neurons connects to the dendrite 70 of OMLI output neuron via their respective terminal 78. However, neurons output and read data counter clockwise, that is from right to left. Now, terminal 78 of query neuron 94 connects to dendrite 70 of solution neuron 92. Then, terminal 78 of solution neuron 92 connects to dendrite 70 of primary neuron 90. Next, terminal 78 of primary neuron 90 connects to dendrite 70 of registry neuron 96. Finally, terminal 78 of registry neuron 96 connects back to dendrite 70 of query neuron 94 in a three-dimensional diamond shaped neuronal memory block 116.

FIG. 21 is a block diagram of a neuronal memory block 116. This theoretical diamond shape creates a uniform memory structure for faster data storage and access. There are four neurons on the perimeter, four across the center, four on the left side of center, and four neurons on the right side of center of the diamond shaped memory block. The dotted gray lines indicate the diamond data flow. The flows are from 112 to 96,90,92, and 94, from 96 to 114, from 90 to 114, from 92 to 114, from 94 to 114, from 94 to 92, from 92 to 90, from 90 to 96, and from 96 to 94 to complete the diamond data flow.

FIG. 22 is one embodiment of tactile data stream 42 and abstract data stream 44. Tactile data stream 42 and abstract data stream 44 are output conduits. Both 42 and 44 can output in stereophonic or monophonic mode, to one or both auricular senses or other tactile sensors. For this embodiment, quantum index 10 is in first position in tactile data stream 42. Natural neuronal buffer 54 separates quantum index 10 from quantum data filter 32, which is in second position. Together, 10 and 32 constitute a QMF audio output 136. Liminal threshold 46 separates the audible tactile data stream 42 from the inaudible abstract data stream 44. The decibels and amplitude of ascending threshold 48 and descending threshold 50 may be adjusted to preserve the absence of abstract data stream 44 at liminal threshold 46. Abstract data stream 44 is an exact duplicate of tactile data stream 42, and both data streams 42 and 44 are synchronized to output simultaneously.

FIG. 23 is another embodiment of tactile data stream 42 and abstract data stream 44. Again, tactile data stream 42 and abstract data stream 44 are output conduits. Both 42 and 44 can output in stereophonic or monophonic mode, to one or both auricular senses or other tactile sensors. In this exemplar, named neuronal registry 54 is in first position. A natural neuronal buffer 52 separates 54 from quantum index 10, which is in second position. Another natural neuronal buffer separates 10 from quantum data filter 32, which is in third position. Together, 54,10, and 32 constitute a QMF audio output 136. Liminal threshold 46 separates the audible tactile data stream 42 from the inaudible abstract data stream 44. The decibels and amplitude of ascending threshold 48 and descending threshold 50 may be adjusted to preserve the absence of abstract data stream 44 at liminal threshold 46. Abstract data stream 44 is an exact duplicate of tactile data stream 42, and both data streams 42 and 44 are synchronized to output simultaneously.

FIG. 24 is still another embodiment of tactile data stream 42 and abstract data stream 44. Again, tactile data stream 42 and abstract data stream 44 are output conduits. Both 42 and 44 can output in stereophonic or monophonic mode, to one or both auricular senses, one or both ocular senses, or other tactile sensors. In this exemplar, named neuronal registry 54 is in first position. A natural neuronal buffer 52 separates 54 from quantum index 10, which is in second position. Another natural neuronal buffer separates 10 from quantum data filter 32, which is in third position. Together, 54,10, and 32 constitute a QMF audio output 136. There is a second signal present in tactile data stream 44. Parallel to the QMF audio output 136 is a QMF video output 138 stream. The QMF video output 138 is synchronized with QMF audio output 136. That is, images or image sequences in 138, may replace and or reinforce data transmitted to or stored in 12-12.d, 14-14.d, 16-16.d, 18-18.d, 20-20.d, 22-22.d, 24-24.d, 26-26.d, 34-34.d, 36-36.d, 38-38.d, 40-40.d, and some or all of the data at 56, 58, 60, 62, 54.A, 54.B, 54.C, and 54.D. Liminal threshold 46 separates the audible tactile data stream 42 from the inaudible abstract data stream 44. The decibels and amplitude of ascending threshold 48 and descending threshold 50 may be adjusted to preserve the absence of abstract data stream 44 at liminal threshold 46. Abstract data stream 44 is an exact duplicate of tactile data stream 42, and both data streams 42 and 44 are synchronized to output simultaneously.

Another embodiment of FIG. 22, FIG. 23, and FIG. 24 is the stacking of abstract data stream 44 in a plurality of row-like configurations.

In another embodiment of FIG. 24, QMF video output 138 exists in tactile data stream 42 and abstract data stream 44.

In still another embodiment of FIG. 24, QMF video output 138 exists only in abstract data stream 44.

In another embodiment of FIG. 22 or FIG. 23, either tactile data stream 42 or abstract data stream 44 may be excluded.

In yet another embodiment of FIG. 22 or FIG. 23, tactile data stream 42 or abstract data stream 44 may consist of a parallel QMF video output 138.

Another embodiment of FIG. 22 or FIG. 23 may consist of tactile data stream 42 without quantum data filter 32 or abstract data stream 44 without quantum data filter 32 or 44 with 138, but without 32 or 42 without 32 but with 138.

Other embodiments, configurations, and arrangements of FIG. 22, FIG. 23, and FIG. 24 are possible and additional embodiments are possible.

FIG. 25 is a block diagram of the Quantum Memory Fusion process in which QMF audio input 126 or QMF video input 128 of a Quantum Memory Fusion recording enter computer 133. Computer 133 is a computing device equipped with linear multi-track audiovisual recording software. Some examples of such software include, but are not limited to Adobe Audition by Adobe corporation or Media studio Pro by ulead corporation. Computer 133 outputs sound to a user at speaker 130 or headphones 150 or other output device. Monitor 132 accomplishes visual output. The processing speed and memory requirements of computer 133 depend on the audiovisual software installed and are therefore not listed as reference figures. Quantum Memory Fusion in textual form may be printed from computer 133 to printer 134. From printer 134, printed matter may be used as publications 170 or transmitted across internet portal 168 in a number of formats to include electronic mail, static web pages, active web pages, or databases. Notwithstanding, QMF audio output 136 may be recorded to QMF audio recorded media such as audiocassette tapes, video cassette tapes, compact discs, or digital video discs. QMF audio output may be used in headphones 150, computer speaker 152, environmental speaker system 154, radio broadcast 156, television broadcast 158, games 160, toys 162, compact disc player 164, digital video disc player 166, or transmitted via internet portal 168. QMF audio output 136 and QMF video output 138 join at QMF audio video output 140. Next, 140 records to QMF audio video recorded media 142 and may be distributed at QMF marketable product 148. QMF video output 138 may be recorded at QMF video recorded media 146 and distributed at 148 via 158, 160, 162, 166, or 168.

FIG. 26 is a drawing of kelly's query funnel 118. Input topics 120-120.e are introduced into the funnel. The paradigm is that kelly's query funnel 118 represents the body of knowledge requisite to known, but difficult tasks. Using Quantum Memory Fusion to increase this body of knowledge, data from a range of topics specific to a query 122 are fused onto memory. NOW, query 122 is posed against this knew knowledge. Query 122 is an unanswerable question. Quantum Memory Fusion assembles and structures the input so that a solution 124 is possible. Quantum Memory Fusion forces the mind to return possible solutions to the unanswerable query 122 based on the input topics exemplified in 120-120.e. The input topics are in now way to be construed as limiting or limited to those input topics depicted herein. There are thousands of possible input subjects available to a user of kelly's query funnel 118.

In another embodiment of FIG. 26, query 122 can enter kelly's query funnel 118 before input topics 120-120.e enter 118.

In still another embodiment, query 122 can enter kelly's query funnel 118 at the same times as input topics 120-120.e.

The method of the invention is also useful in the following areas:

Experimental and clinical uses of psychodynamic, gestalt, cognitive-behavioral, and self-psychology conceptualizations for assessment, prevention, and treatment of psychiatric conditions (memory of childhood abuse, failure patterns, and phobic responses).

Biofeedback, hypnosis, neurology (stroke, concussions), opthalmology, conceived induction states for the purpose of treatment for pain control (e.g., headaches), and their management and relief.

Assessment, prevention, and treatment (including induction states) are achieved with visual, auditory, and tactile sensory stimulation with Quantum Memory Fusion and Quantum Fusion Formulas.

Quantum Memory Fusion can be used in any environment wherein behavioral modification is important in the environment. Quantum Memory Fusion can implant “age-old” memories of relaxation and euphoria in high anxiety patients. Other possible applications include pain management and abatement in postoperative surgical environments.

### OPERATION

1. Data Formatting and Assignments

Tables 4-1-4-3 exemplify Quantum Memory Fusion data prepared for recording. The QMF format shown in the above tables is equally successful as written output. Therefore, text written and distributed using this format will fuse to permanent recallable memory faster than any known textual format. For the sake of explanation, this detailed exemplar of Quantum Memory Fusion focuses on the use of audible data. Visual data may be used. That is, images or image sequences may replace and or reinforce data transmitted to or stored in 12-12.d, 14-14.d, 16-16.d, 18-18.d, 20-20.d, 22-22.d, 24-24.d, 26-26.d, 34-34.d, 36-36.d, 38-38.d, 40-40.d, and some or all of the data at 56, 58, 60, 62, 54.A, 54.B, 54.C, and 54.D. A plurality of rows of data may be used and a plurality of audio, visual and or audiovisual data may be used to replace and or reinforce the data transmitted to or stored in said plurality of rows of data.

Table 4-1 depicts named neuronal registry 54 as shown in FIG. 15. In Table 4-1, I assigned data to 56-62. Factors 4-1.a-4-1.h itemizes the data assignments.

4-1.a. registry name soma 56 contains “American.”

4-1.b. registry data dendrite 58 contains “Presidents.”

4-1.c. registry key dendrite 60 contains “First.”

4-1.d. registry index nucleus 62 contains “Ladies.”

4-1.e. spatially aware dendrite 54.A is empty.

4-1.f. spatially aware dendrite 54.B is empty.

4-1.g. spatially aware dendrite 54.C is empty.

4-1.h. spatially aware dendrite 54.D is empty.

Following 62, there can be a natural neuronal buffer 52 of an amount of silence that ranges in duration Fifteen seconds of 52 is ample. During the range of 52, the brain supplies environmental variables to complete 54. The environmental variables can be more or less, than the number of dendrites indicated in Table 4-1, items 54.A-54.D. The environmental variables may be anything that the brain deems important at the moment a QMF session begins.

An audio recording is produced using the data in 54. The data in 54 is recorded in the native language of the user. NOW, registry neuron 96 will receive data during fusion.

Table 4-2 depicts quantum index 10 shown in FIG. 1. Factors 4-2.a-4-2.h itemizes the data assignments.

4-2.a. primary ordinal soma 12 contains “First.”

4-2.b. primary data dendrite 14 contains “President.”

4-2.c. primary key dendrite 16 contains “George.”

4-2.d. primary index nucleus 62 contains “Washington.”

4-2.e. solution main soma 20 contains “George.”

4-2.f. solution data dendrite 22 contains “Married.”

4-1.g. solution key dendrite 24 contains “Martha.”

4-2.h. solution index nucleus 26 contains “Washington.”

Following 26, there can be a natural neuronal buffer 52 consisting of an amount of silence that ranges in duration. Five seconds of 52 is ample.

An audio recording is produced using the data in 10. The data in 10 is recorded in the native language of the user. Now, primary neuron 90 and solution neuron 92 will receive data during fusion.

Table 4-3 depicts quantum data filter 32 shown in FIG. 11. Factors 4-3.a-4-3.d itemizes the data assignments.

4-2.a. filter query soma 34 contains “1”

4-2.b. filter data dendrite 36 contains “Who.”

4-2.c. filter key dendrite 38 contains “Married.”

4-2.d. filter index nucleus 40 contains “Washington.”

An audio recording is produced using the data in 32. The data in 32 is recorded in the native language of the user. NOW, query neuron 94 will receive data during fusion.

2. Masking Data Streams

My exhaustive experimentation proves that musical masks provide no benefit in Quantum Memory Fusion. Conversely, musical masks appear to create additional neuronal memory blocks of unknown configuration and create mnemonic diversions that inhibit the cognizant recitation response. The harmonics present in musical masks interfere with dendritic-axonic-harmonic-output 108, thereby defeating its purpose. Certain segments of the musical masks are unrecognizable chunks of information in the dendritic data directories, such as at natural neuronal buffer 52, when the masks' output occupied the duration of 52, consolidating pre 52 data with post 52 data. I see no reason to risk fusion performance with unnecessary harmonics, especially when fusion occurs without the user being relaxed, or being aware of the data, or even paying attention during the session.

3. Data Relationships and Operation

Tactile data stream 42 in stereophonic representation is the method of delivery discussed hereafter. The data stored in 54 closely relates to the data in 10 as the data in 54 describes the data in 10. This descriptive relationship helps 96 quickly respond to queries referencing data in 92. The data in 10 closely relates to the date in 32.

In Tables 4-2 and 4-3, primary index nucleus 18, solution main nucleus 26, and filter index nucleus 40 each contain the word “Washington” in the data field. Like any database (whether flat file or relational), the information in the data fields must share at least one common index to be retrievable and neuronal memory block 116 is an organic database. Because “Washington” is the name of each nucleus, quantum data filter 32 can specifically target all nuclei in 116 in approximately 500 milliseconds as proven in my research and experiments. However, it is not a specific requirement of quantum data filter 32 to hold “Washington” in filter index nucleus 40. I have also found that if the index appears anywhere in 32 or if the data from solution main soma 20 or solution data dendrite 22 is present in any field in 32, quantum data filter 32 will produce tangible results. Nevertheless, it is a strict requirement for primary index 28 and solution index 30 to share the same index.

The data in solution index 30 is important to fusion because the answer to the query is in 30 at solution key dendrite 24. Solution key dendrite 24 is the dendrite that holds the data that 32 will query. Throughout my research, data in 24 was the recallable data immediately following fusion. The other data in quantum index 10 reinforces solution key dendrite 24 and becomes recallable to the degree that the data relates back to 24. Fusing 24 for recall fuses all of the data in 10, 32, and 54. However, 32 and 54 are not recallable, but permanent.

Once queried by 32, that solution index nucleus 26 reads backwards until it encounters solution main soma 20. Solution main soma 20 extracts and delivers all data between 20 and 26 to the OMLI output neuron 114 as shown in FIG. 20. That is, solution main soma 20 sends the data in solution data dendrite 22 and solution key dendrite 24 to 114 for output. The words “Married Martha” are converted from organic machine language to the native language of that brain and passed out of 114 to a neural relay that targets the speech center of the brain. However, because query neuron 94 forced registry neuron 96 to reach neuro critical mass artificially, the output is amplified at the harmonic synaptic cleft 110 of OMLI output neuron 114. An amplified bio-electro-harmonic vibration results at dendritic-axonic-harmonic-output 108. Like any electromagnetic coil, the bio-electro-harmonic resonance reproduces vibrations that “sound like” the amplified data retrieved from solution key dendrite 24, in a harmonic voice inside the brain that is “heard” by the individual.

During fusion, the data stored in solution key dendrite 24 is the instantly recallable data and the balance of data in 10 is permanent and recallable after time. The data stored in 24 can be a single word, numbers, or a phrase so long as the natural neuronal buffer 52 between words does not exceed 500 milliseconds (+ or −20%). If 52 exceeds the maximum duration, remaining data may spill-over to solution index nucleus 26 and the entire data set in 10 will create permanent, but not recallable memory. Lack of data indexes and information spill-over illustrates the current neuronal model of traditional learning, which is why traditional learning is slow and inefficient. My research indicates that an unlimited stream of data may be stored in solution key dendrite 24. Once solution key dendrite 24 nears its maximum data capacity, other dendrites or possibly other neurons will assist in data storage as shown in FIG. 10. When solution neuron 92 stores data in other neurons, 92 will create the soma and nuclei names of the additional neurons. The individual has no knowledge of these names. However, solution neuron 92 has direct axonic links to each new neuron.

4. Abstract Data

FIG. 23 is representative of Tables 4-1-4-3 insofar as structure is concerned. Thus far, Quantum Memory Fusion used tactile data stream 42 for a consciously audible version of fusion. Because I have copiously redefined and dispelled myths about, and explained the exact nature of abstract content and methodology, I will address the raw power of its use in Quantum Memory Fusion. I have established that abstract content will only produce a response if some form of substantiated data exists to guarantee the brain that it is not acting on fantasy or phantom constructs. In other words, the data must be cognizant at the immediate moment or some version of the data must reside in the cache of knowledge of that brain. Below, I illustrate three additional discoveries I made during testing.

First, the contents of abstract data stream 44 bypasses any possible filtering by the left hemisphere of the brain and create neuronal activity in the right hemisphere of the brain. The neuronal activity occurs during waking or sleeping sessions.

Second, if any portion of the data in tactile data stream 42 already exists in the cache of knowledge of that brain, a neuronal memory block 116 will form based on the delivery of data in abstract data stream 44 alone. My research proved that when abstract data stream 44 queries the cache of knowledge, that query neuron 94 returned verifiable answers present in the inaudible abstract data stream 44.

Third, when abstract data stream 44 is present alone, the test subjects were free to engage in other activities to include watching television, drawing pictures, coloring, eating and even sleeping. Nevertheless, the test subjects were able to render up to 80% cognizant recitation of the unknown content, indicating just how much of the content existed in the internal cache of knowledge of the test subject. These results further substantiated my theory that every event or stimulus encountered in the lifetime of a brain becomes permanent memory if it receives attention.

5. Validating Abstract Data

Abstract data should be validated upon cognizant recitation of a correct response. Although the abstract data may have substantial support within other neuronal memory blocks of the cache of knowledge of that brain, the fact remains that a user never hears the data, answer, or question during a QMF session with abstract data stream 44 only. I discovered that by validating a cognizant recitation with phrases such as “That is correct!” and “Great Job!” my test subjects showed higher percentages of correct answers than when I used phrases such as “Ok” and “Next question.” I attribute the higher percentage to the fact that validation helped the test subjects recognize the feeling internally associated with the correct answer. Remember that feelings are stored in registry neuron 96 at the beginning of the session as an environmental variable. Future feelings about the neuronal memory block are also stored in registry neuron 96 when they occur. I further noted that when I did not validate a correct response, some of the test subjects stated that they did not “feel” as though their correct response was correct. Finally, I attribute the “feeling” of incorrectness reported by test subjects to the test subjects having not heard the data fused to their memory.

6. Tactile and Abstract Data Combined

When tactile data stream 42 and abstract data stream 44 contain identical data and transmit that data simultaneously to a user, the results of fusion become immediately apparent (usually within sixty minutes). Because 42 is cognizant, it is subject to filtering by the left hemisphere of the brain. Filtering can be the result of innumerable issues, including indifference to the data or anxiety about the content (as in mathematics). However, the negative probability of filtering spawned yet another discovery. The abstract content is considered substantiated at the instance both hemispheres of the brain receive the same version of the data, thereby bypassing the age-old practice of months and years of agonizing repetition in learning. Consequently, abstract data stream 44 is elevated from the realms of fantasy and phantom constructs to verified, permanent and recallable memory. The left hemisphere of the brain is powerless to intervene even if it has already filtered and rejected tactile data stream 42. The incognizant data in abstract data stream 44 adheres to the cognizant data in tactile data stream 42 whether the left brain hemisphere likes it or not. The result is that 44 becomes “real” and 42 is dismissed and fusion occurs. Furthermore, the memories fused by this process do not feel “new,” instead, test subjects reported that they felt that they had always known the information. These results substantiate yet another of my discoveries. The new neuronal memory block 116 will create many spontaneous connections to other neuronal memory blocks. Its goal is to strengthen its own data and guarantee the survival of its neurons and the precious cargo in its dendrites. Other neuronal memory blocks will battle for prime synaptic connections to the new data rich resources being announced across the neural network. Quantum Memory Fusion exploits the survival strategy of neuronal memory blocks, while assisting and strengthening the neural network.

7. Audible Query Versus Sub-Mnemonic Query

The query against solution index 30 resides in quantum data filter 32. Although the query is not required to induce fusion, the query serves a valuable purpose.

When 32 is audible, we risk query rejection because the answer is unknown. However, when in abstract, we produce a sub-mnemonic query against solution index 30. The left hemisphere of the brain is incapable of filtering the sub-mnemonic query. It is well established that the left hemisphere of the brain can only service cognizant data and stimulus. The sub-mnemonic query does not constitute phantom data because it has as its index, the same data present in the indexes of primary index nucleus 18 of primary index 28 and solution index nucleus 26 of solution index 30. The index represents substantial evidence of the validity of the data. Because the sub-mnemonic query is abstract, the right hemisphere of the brain can devote its full problem-solving powers to seeking the solution. Once the solution is found, the solution itself will cause neurons from other neuronal memory blocks to attach to the newly found data. The solution is now permanent and recallable and the test subject never knew that they did not yet know the answer because they never heard the question.

8. Recitation Query Versus Action Query

The output of a query depends on the results expected. There are at least two types of queries used in Quantum Memory Fusion. The recitation query is the easiest to use because the result is usually a verbal response indicating a solution to data fused to permanent, recallable memory. Table 4-3 depicts a recitation query, while Table 4-2 shows the relationship of the recitation query to the data.

The second type of query is the action query. The action query results in neuromuscular response or behavioral response. Behavior modification action queries work best when the resulting behavioral response is a desired response of the user. Nevertheless, any data fused in the Quantum Memory Fusion method will result in the outcome of the data design. An action query may use external stimulus as the query or an internal process as the query.

Table 4-5 is an exemplar of an action query, while Table 4-4 shows the relevance of the action query in Table 4-5 to the data in Table 4-4. The action query in Table 4-5 results in behavioral modification.

Table 4-7 is another exemplar of an action query, while Table 4-6 shows the relevance of the action query in Table 4-7 to the data in Table 4-6. The action query in Table 4-7 results in neuromuscular response.

An unobvious advantage of the queries in Table 4-7 and Table 4-5 is that the data in both queries exist in the cache of knowledge of the brain to which the query is posed. Therefore, the queries can be abstract because substantial evidence exists in other neuronal memory blocks that prevent the queries from being construed as mere fantasies. The same applies to Table 4-4 and Table 4-6.

Another unobvious advantage of the action query is that it produces unconscious obedience. I termed this obedience “reflexive memory recall” because an external agent or stimulus never queries the actual memory. Instead, the neuronal memory block queries itself when the individual initiates an activity resembling the query.

9. Using Cardinal and Ordinal Numbers

During many experiments and tests, I created hundreds of variations of the data in primary ordinal soma 12 of primary index 28, of quantum index 10, of primary neuron 90. What I discovered is that neuronal memory block 116 does not automatically make primary neuron 90 the first neuron after registry neuron 96. Ultimately, this makes perfect sense because registry neuron 96 is still a neuron. If its dendrites reached capacity, then it would simple call another neuron to its terminals. However, by naming the soma of primary neuron 90 with a cardinal or ordinal number instead of an alpha character, primary neuron 90 moves to a right adjacent position to begin the formation of the center of the diamond structure. However, cardinal and ordinal numbers had no effect on solution neuron 92 and query neuron 94. In every experiment, solution main soma 20 had to be different from filter query soma 34 in order to produce cognizant recitation response, but neither solution main soma 20 nor filter query soma 34 contained a number.

When using alpha characters in primary soma 12, the result is that another registry neuron 96 is called to the diamond structure instead of primary neuron 90. Permanent recallable memory will permit the recall of some data in a registry neuron. However, if the diamond structure is to form, the next phrase must contain a cardinal or ordinal number. Cardinal or ordinal numbers in primary ordinal soma 12 from the beginning of the transmission yields the better results.

Preceding the cardinal numbers with the word “number” places the word “number” in primary soma 12, not the ordinal number. The ordinal number moves to primary data dendrite 14.

Another important discovery about the use of numbers with neurons is that certain numbers will count as two words not just a single word number. Tables 4-8 and 4-9 illustrate this interesting anomaly that proved to be a very important discovery. Table 4-8 is suffixed ordinal numbers. Table 4-9 is cardinal numbers.

In Table 4-8 and Table 4-9, numbers that consist primarily of a one's column or a single ten's column and a one's column, occupy primary ordinal soma 12 only. When the number consists of two ten's in the ten's column and at least a single one in the one's column, then the one's column occupies primary data dendrite 14. Even when recorded without a minimum natural neuronal buffer 52, primary neuron 90 allocates the numbers to columns, which is actually an allocation to its soma and then a dendrite. This numbering schematic applies to all neurons regardless of positioning in a neuronal memory block 116

When using numbers, I highly recommend that the technician or user responsible for creating the fusion data spell-out the numbers according to Tables 4-8 and 4-9.

10. Cardinal and Ordinal Number Data Grouping

Another discovery was that any number used as data in primary ordinal soma 12 could be identical to the numbers in other primary ordinal somas in the neuronal memory block as shown in FIG. 5. Primary ordinal somas 12-12.a have the effect of writing all five rows of data to the same primary neuron. The caveat being that primary index nucleus 18 would be over-written when 18.a fused, and 18.a would be over-written when 18.b fused, and 18.b would be over-written when 18.c fused, and 18.c would be over-written when 18.d fused, and 18.d would be over-written by 18. This cycle would continue for the duration of the QMF session.

By way of example, if 12-12.a contained the ordinal number “tenth” and 14-14.d each contained a word “house, cat, dog, mouse, bird” respectively, then primary index 28 would have five named dendrites, but only a single soma named “tenth.” If primary index nuclei 18-18.d each contained a word “boat, food, bone, trap, bath” respectively. The first fused item is “house boat.” A dendrite records “house” into permanent memory, but “boat” will be replaced. The second fused item is “cat food.” A dendrite records “cat” into permanent memory, “food” replaces “boat,” but “food” will also be replaced. The third fused item is “dog bone.” A dendrite records “dog” into permanent memory, “bone” replaces “food,” but “bone” will be replaced. The fourth fused item is “mouse trap.” A dendrite records “mouse” into permanent memory, “trap” replaces “bone,” but “trap” will be replaced soon. The fifth fused item is “bird bath.” A dendrite records “bird” into permanent memory, “bath” replaces “trap,” but “bath” will be replaced when the fusion cycles.

This method is important in that it completely over-writes the soma name on each rotation and allows many items to fuse to a single neuron. I discovered that I can create smaller neuronal memory blocks with greater data compression and faster mnemonic reading. Any data in primary neuron 90, solution neuron 92, query neuron 94, or registry neuron 96 can be grouped or over-written in this method.

11. Quantum Index Data Grouping for Solution Index

FIG. 9 illustrates a perfect example of data grouping. Data grouping is useful when no specific data item is required and any data item in the group will produce the required result. Table 4-10 provides data for FIG. 9's grouping method. Primary somas 12-12.d are unique. Thus, there are five primary neurons labeled 90-90.d. Solution main somas 20-20.d create a single solution main soma. Therefore, there is only one solution neuron 92. Solution data dendrites 22-22.d create one solution data dendrite. Solution index nucleus 26-26.d are unique and solution key dendrites 24-24.d are unique.

The most important rule for data grouping is caution. Data grouping is quite similar to data erasure. The reason we can group data without erasing the previous data is solution index nucleus 26. Through countless experiments, I discovered that the nucleus of a neuron behaves differently from the soma, whereas you can overwrite the name of a soma, you can not overwrite the name of a nucleus. The nucleus of a neuron keeps an ordered list of its designations (in this case names) for the life of the neuron. This powerful discovery is the quintessential wonder of permanent recallable memory. The nucleus is the switch that transmits data from a dendrite that the soma references upon query, down the axon to the appropriate terminal. The nucleus may also command the axon to grow a new terminal. The nucleus is a literal cpu and router in one microscopic organic device.

In Table 4-10, solution key dendrites 24-24.d. Respectively contain the words “weight, fat, pounds, sizes, inches” in the data fields. Solution index nucleus' 26-26.d respectively contain the words “salad, fish, fruit, vegetables, soy” in the data fields. As QMF audio output 136 transmits, a single neuronal memory block 116 forms. Then, five primary neuron 90 join 116. However, only one solution neuron 92 joins 116. Solution neuron 92 will store data from 24-24.d on five separate dendrites, while naming solution index nucleus 26-26.d five separate times.

When the action query in Table 4-11 is posed using the word “salad” in filter index nucleus 40, solution index nucleus 26 will respond. However, because solution index nucleus 26 has four other names, it will not merely seek its own solution key dendrite 24, it will read backwards from each solution index nucleus 26-26.d up to solution main soma 20 and retrieve whichever item it wants, and in some experiments, it retrieved all of the items from solution key dendrites 24-24.d. In those experiments, the test subject would recite the data from the solution key dendrites without matching the bulk of the data to the appropriate solution index nucleus. According to solution index nuclei 26-26.d, any data on solution key dendrites 24-24.d is acceptable because all of the data is stored in a single solution neuron 92. Moreover, filter index 40 could use any of its other possibilities such as “fish, fruit, vegetables, soy” and the result would be the same. My experimentation showed that eventually, each nucleus would mate with the appropriately named dendrite. Nonetheless, the neuron performs as intended, as a cache for interchangeable solutions.

12. Updating and Erasing Data in Neurons

Updating permanent recallable memory at the neuronal level is just as easy as fusing the original memory. If the technician or user desires to update only a dendrite, then he or she need only edit out the old data from the recording and record the new data in its place. Using as much of the previous data harmonics as possible is important so as not to create an entirely new neuronal memory block 116. If the word “Danger” in primary key dendrite 16, shown in Table 4-4 is the target for update, edit out the word “Danger” from the QMF audio output 136. Record the word “Cancer” and Replace “Danger” with the “Cancer” recording. Re-render QMF audio output 136 and start a QMF session. Because the remaining data in quantum index 10 is unchanged, the first dendrite becomes the first available dendrite although the dendrite is not empty. The word “Cancer” over-writes the previous stored word “Danger” on primary key dendrite 16.

The same process is used to erase data. Instead of recording the word “Cancer,” record a natural neuronal buffer 52 of approximately 700 milliseconds. Now, “Danger” is erased but the diamond structure remains intact.

Repetition is helpful to erase data because primary ordinal soma 12 acts as a “system registry” for the entire neuron. The soma of a neuron automatically creates references to the data stored in its dendrites. Primary soma 12 created reference pointers to primary key dendrite 16 that previously contained the word “Danger.” Repetitiously re-writing the soma will remove the pointers to the non-existent data, a practice equivalent to deleting invalid file references from the registry database on a personal computer.

13. Data Stream Stacking

Data Stream stacking is the process of creating multiple synchronized abstract streams of the same or different information that varies in amplitude, decibel volume, pitch, or frequency. Data stream stacking is possible because of Neurothreading. When information is available in the cache of knowledge of the brain that will substantiate data in an abstract data stream 44, multiple instances of abstract data stream 44 are possible. Each instance may use a different voice, or pitch or frequency so that it is unlike other abstract streams. The abstract streams may contain the same data or a plurality of unique programs. The results from my experimentation show that an abstract stream of synchronized information creates a single neuronal memory block, while unique information streams create new neuronal memory blocks. Table 4-12 illustrates a data stream stacking example containing like information on twenty channels with 20 different frequencies. Table 4-13 illustrates a data stream stacking example that exploits neurothreading with unique information in the data stream stacks on two separate channels.

Data stream stacking offers twenty times more fusion per session, with acceptable amounts of neuronal discord.

14. Solution Key Dendrites as High Speed Portals

My experimentation proved that quickly spoken words, which normally constitute multiple words, such “OneHundredFortyFour” record to a single dendrite. The organic machine language interpreter (OMLI input neuron 112) interpreted the data stream with no difficulty and slowed the fast-spoken words to normal speaking speed before writing the stream to solution key dendrite 24. This discovery led to another discovery that I termed “Neural Data Streaming,” which is the process of locking a dendrite into a continuous stream of audible, visual, tactile or abstract data. I theorize that a 300 page book, with 200 words per page, normally read at 200 words per minute could be fused to permanent recallable memory within sixty minutes. The book has 60,000 words total and a reader would normally need five hours to read the book. If recorded at 200 words per minute, and sped up to 5 times normal speed, the audio would transmit at 1,000 words per minute. The text displays in a scrolling method at solution key dendrite 24. The text is tactile, the audio is abstract, and the transmission synchronized. Theoretically, the contents of the book would write to permanent recallable memory in one hour, with every word of the text recallable upon query. The probability that other dendrites would behave similarly certainly exists. The possibilities are endless for this application in global commerce and education.

15. Wording Fusion Programs

The brain does not analyze sensory input for grammatical correctness before storing the input. In fact, the brain is unconcerned about the human language structure. The brain existed before spoken language existed and that is the clue, not the anomaly. The words and word order are important only to the degree that they lead to a shared index among the neurons and an answer in solution key dendrite 24. The brain will use its learned language skills to recreate an ordered structure. Finally, the query is helpful, but not necessary to produce results.

TABLE 4-1
Named Neuronal Registry Data

| 56 | 58 | 60 | 62 | 54.A | 54.B | 54.C | 54.D |
|---|---|---|---|---|---|---|---|
| American | Presidents | First | Ladies | | | | |
| IMAGE | IMAGE | IMAGE | IMAGE | Empty | Empty | Empty | Empty |

TABLE 4-2
Quantum Index Data

| 12 | 14 | 16 | 18 | 20 | 22 | 24 | 26 |
|---|---|---|---|---|---|---|---|
| First | President | George | Washington | George | Married | Martha | Washington |

TABLE 4-3
Quantum Data Filter Data

| 34 | 36 | 38 | 40 |
|---|---|---|---|
| One | Who | Married | Washington? |

TABLE 4-4
Quantum Index Data - Behavior Modification

| 12 | 14 | 16 | 18 | 20 | 22 | 24 | 26 |
|---|---|---|---|---|---|---|---|
| First | Death | Danger | Cigarettes | I | Quit | Smoking | Cigarettes |

TABLE 4-5
Action Query Data - Behavior Modification

| 34 | 36 | 38 | 40 |
|---|---|---|---|
| When | Reaching | For | Cigarettes? |

TABLE 4-6
Quantum Index Data - Neuromuscular Response

| 12 | 14 | 16 | 18 | 20 | 22 | 24 | 26 |
|---|---|---|---|---|---|---|---|
| Second | Ciga- | Cause | Pain | When | Smelling | Cigarettes | pain |
| | rettes | | | | | | |

TABLE 4-7
Action Query Data - Neuromuscular Response

| 34 | 36 | 38 | 40 |
|---|---|---|---|
| What | Causes | Me | Pain? |

TABLE 4-8
Ordinal Numbering with “th” and “eth” suffixes - up to 30

| 12 | 14 | 16 | 18 |
|---|---|---|---|
| First | | | |
| Second | | | |
| Third | | | |
| Fourth | | | |
| Fifth | | | |
| Sixth | | | |
| Seventh | | | |
| Eighth | | | |
| Ninth | | | |
| Tenth | | | |
| Eleventh | | | |
| Twelfth | | | |
| Thirteenth | | | |
| Fourteenth | | | |
| Fifteenth | | | |
| Sixteenth | | | |
| Seventeenth | | | |
| Eighteenth | | | |
| Nineteenth | | | |
| Twentieth | | | |
| Twenty | First | | |
| Twenty | Second | | |
| Twenty | Third | | |
| Twenty | Fourth | | |
| Twenty | Fifth | | |
| Twenty | Sixth | | |
| Twenty | Seventh | | |
| Twenty | Eighth | | |
| Twenty | Ninth | | |
| Thirtieth | | | |

TABLE 4-9
Cardinal Numbering - up to 30

| 12 | 14 | 16 | 18 |
|---|---|---|---|
| One | | | |
| Two | | | |
| Three | | | |
| Four | | | |
| Five | | | |
| Six | | | |
| Seven | | | |
| Eight | | | |
| Nine | | | |
| Ten | | | |
| Eleven | | | |
| Twelve | | | |
| Thirteen | | | |
| Fourteen | | | |
| Fifteen | | | |
| Sixteen | | | |
| Seventeen | | | |
| Eighteen | | | |
| Nineteen | | | |
| Twenty | | | |
| Twenty | One | | |
| Twenty | Two | | |
| Twenty | Three | | |
| Twenty | Four | | |
| Twenty | Five | | |
| Twenty | Six | | |
| Twenty | Seven | | |
| Twenty | Eight | | |
| Twenty | Nine | | |
| Thirty | | | |

TABLE 4-10
Quantum Index Data Grouping for Solution Index

| Row | 12 | 14 | 16 | 18 | 20 | 22 | 24 | 26 |
|---|---|---|---|---|---|---|---|---|
| | First | Exercise | Eat | Salad | I | Shed | Weight | Salad |
| a | Second | Exercise | Eat | Fish | I | Shed | Fat | Fish |
| b | Third | Exercise | Eat | Fruit | I | Shed | Pounds | Fruit |
| c | Fourth | Exercise | Eat | Vegetables | I | Shed | Size | Vegetables |
| d | Fifth | Exercise | Eat | Soy | I | Shed | Inches | Soy |

TABLE 4-11
Action Query Data - Behavior Modification

| 34 | 36 | 38 | 40 |
|---|---|---|---|
| Can't | I | Eat | Salad? |

TABLE 4-13
Abstract Data Stream Stacking - Synchronized

| Channel | | | | | | | | |
|---|---|---|---|---|---|---|---|---|
| Frequency | 12 | 14 | 16 | 18 | 20 | 22 | 24 | 26 |
| A - 20 kHz | Second | Cigarettes | Cause | Pain | When | Smelling | Cigarettes | Pain |
| B - 19 kHz | Second | Cigarettes | Cause | Pain | When | Smelling | Cigarettes | Pain |
| C - 18 kHz | Second | Cigarettes | Cause | Pain | When | Smelling | Cigarettes | Pain |
| D - 17 kHz | Second | Cigarettes | Cause | Pain | When | Smelling | Cigarettes | Pain |
| E - 16 kHz | Second | Cigarettes | Cause | Pain | When | Smelling | Cigarettes | Pain |
| F - 15 kHz | Second | Cigarettes | Cause | Pain | When | Smelling | Cigarettes | Pain |
| G - 14 kHz | Second | Cigarettes | Cause | Pain | When | Smelling | Cigarettes | Pain |
| H - 13 kHz | Second | Cigarettes | Cause | Pain | When | Smelling | Cigarettes | Pain |
| I - 12 kHz | Second | Cigarettes | Cause | Pain | When | Smelling | Cigarettes | Pain |
| J - 11 kHz | Second | Cigarettes | Cause | Pain | When | Smelling | Cigarettes | Pain |
| K - 10 kHz | Second | Cigarettes | Cause | Pain | When | Smelling | Cigarettes | Pain |
| L - 09 kHz | Second | Cigarettes | Cause | Pain | When | Smelling | Cigarettes | Pain |
| M - 08 kHz | Second | Cigarettes | Cause | Pain | When | Smelling | Cigarettes | Pain |
| N- 07 kHz | Second | Cigarettes | Cause | Pain | When | Smelling | Cigarettes | Pain |
| O - 06 kHz | Second | Cigarettes | Cause | Pain | When | Smelling | Cigarettes | Pain |
| P - 05 kHz | Second | Cigarettes | Cause | Pain | When | Smelling | Cigarettes | Pain |
| Q - 04 kHz | Second | Cigarettes | Cause | Pain | When | Smelling | Cigarettes | Pain |
| R - 03 kHz | Second | Cigarettes | Cause | Pain | When | Smelling | Cigarettes | Pain |
| S - 02 kHz | Second | Cigarettes | Cause | Pain | When | Smelling | Cigarettes | Pain |
| T - 01 kHz | Second | Cigarettes | Cause | Pain | When | Smelling | Cigarettes | Pain |

TABLE 4-13
Abstract Data Stream Stacking - Neurothreading

| Channel Voice | 12 | 14 | 16 | 18 | 20 | 22 | 24 | 26 |
|---|---|---|---|---|---|---|---|---|
| A - male | First | Exercise | Lose | Weight | Only | Eat | Salad | Weight |
| B - female | Fifth | President | James | Monroe | First | Lady | Elizabeth | Monroe |

### QUANTUM MEMORY FUSION EXAMPLES

The specific examples below describe exemplary quantum memory fusion samples for possible use in academics, health care, mental health, and self help,

### Academic

### Multiplication by 12

### Example 1

The average secondary school in the united States generally requires that a student know the multiplication facts up to 12, by grade four. The failed enforcement of the basics of multiplication contributes to problems in other academic areas. Approximately 4% of students grades 9-12 drop out of high schools nationwide according to a National center for Education Statistics 2000 survey. Quantum Memory Fusion fuses a complete multiplication fact to permanent recallable memory in sixty minutes or less to a child between 5-7 years of age. Tables E.1 and E.1a is an example of a fusion program for multiplication facts of 12. Column 24 contains cardinal numbers that normally occupy more than a single dendrite. For those numbers to occupy only a single dendrite, the number must be spoken quickly without audible pause. This example fuses effortlessly.

### Health Care

### Weight Loss

### Example 2

Obesity in the United states is an epidemic. In an article in the Sep. 23, 2002 issue of Health currents, an estimated 120 million adults are overweight and 14% of children aged 6 through 19 are severely overweight. Obesity is a global problem however. According to the united Kingdom's National Audit office, 58% of adults in England are overweight or obese. In Russia, 54% of adults are overweight according to the world watch Institute in Washington D.C. The effects of obesity are severe and as many as 300,000 Americans die each year from obesity-related illnesses according to the centers for Disease Control Prevention. There are numerous side effects concomitant to obesity such as lack of confidence, lack of self-esteem, reduction in social connectivity, depression, and poor anger management.

Over-eating and lack of exercise are the two major influences of obesity. Over-eating is a learned response that can be unlearned just like any other conditioned reflex. However, “unlearning” a condition that has become permanent recallable memory is difficult at best. Quantum Memory Fusion eliminates the mental barriers to good eating habits and physical fitness by fusing onto permanent recallable memory the personality construct of a normal healthy person insofar as dietary habits and exercise regimen are concerned. Table E.2 illustrates an example of a program to assist in weight loss.

### Health Care

### Tobacco Aversion

### Example 3

Tobacco use remains the single most preventable cause of death in the united States. More than 400,000 Americans die each year from tobacco related disease. In the united States alone, 48.7 million adults are smokers and approximately 4.1 million teenager's ages 12 through 17 are smokers. The Federal Office on Smoking and Health estimates that 3,000 young people start smoking each day. More than 145 thousand women die of smoking related diseases annually. Tobacco use kills and many smokers make valiant attempts to quit. Approximately 1.3 million smokers quit each year for the 1.09 million that start each year among our nation's youth. Society is making no progress in the battle against tobacco use, addiction, and eventual deaths. Quantum Memory Fusion helps tobacco users fight the urge to smoke by fusing new behavior constructs onto permanent recallable memory.

Nicotine is an addictive substance. Pharmaceutical companies use diluted forms of nicotine to wane tobacco users. However, replacing one chemical dependency with another is counter-productive. Quantum Memory Fusion replaces the desire to smoke with a desire to quit smoking. Quantum Memory Fusion wages a neuronal battle against old memories of euphoria by creating new, healthy memories. Tables E.3-E.3a are examples of a tobacco aversion program and abstract action query.

### Self Help

### Leadership Traits

### Example 4

There are thousands of famous people in the world. Many of them exhibit not only desirable financial skills, but honed and admirable personality traits. Celebrated performance trainers and motivational speakers have tried for nearly three decades to implant some of those principles onto the minds of their followers. Unfortunately, their methods show only minimal success. Napoleon Hill (1883-1970), the author of “Think And Grow Rich” said it best, “what ever the mind of man can conceive and believe, it can achieve.” Therein lies the problem.

For any cognitive stimuli to become a trait or behavior, it must first become a memory. By our evolved nature, humans are pessimistic creatures. Pessimism, skepticism, and disbelief of artificial stimulus will cause the immediate rejection of the proposed memory. Quantum Memory Fusion is the only know methods that ethically bypasses these natural safeguards to give the user the traits he or she desires. Tables E.4-E.4a illustrate a leadership personality construct program based on the 14 leadership traits as taught by the United States Marine corps.

### Academic

### English Vocabulary

### Example 5

Words are the key to communication. When a person has a grasp and understanding of the meanings of many words, studies have shown that those persons have typically higher incomes and positions with greater responsibility. Children need larger vocabularies today than in previous generations because of the consistent advances in technology. The basis for all learning is the ability to read, write, and understand words. In the united states, 10 million adults are deemed illiterate. They are unable to read, write, or perform simple arithmetical problems well enough to function normally in society. Two of the causes for adult illiteracy are poor teaching of basic math and reading in elementary schools and high drop-out rates among certain classes of high school students.

Quantum Memory Fusion helps resolve this problem by fusing the meanings of words onto permanent recallable memory. Table E.5-E.5a exemplify a vocabulary fusion program.

### Academic

### Spanish Vocabulary

### Example 6

The United States is the most demographically diverse nation on earth. There are many languages spoken in the U.S. besides English, to include, Spanish, French, Chinese, and Japanese. Quantum Memory Fusion provides an effortless way to learn foreign words. Tables E.6-E.6a illustrate a Spanish language fusion program.

### Mental Health

### Depression Related to Divorce

### Example 7

An estimated 44.3 million Americans ages 18 and over suffer from a diagnosable mental disorder in any given year according to the National Institute of Mental Health. More than 18.8 million adults age 18 and older suffer from some variation of a depressive disorder. Approximately 19.1 million American adults ages 18 to 54 suffer from an anxiety disorder that frequently co-occurs with depressive disorders, eating disorders, or substance abuse. Divorce is leading cause of depression. Divorce induces anxiety, depression, loss of appetite, and even bitter hostility several years later. In 2000, there were over 21 million divorces according to the Divorce center. Quantum Memory Fusion provides a simple method for psychological management of some of the disorders. Psychiatrist, psychologist, and other psychotherapy professionals can fuse solutions onto the memories of their patients in a one-hour therapy session, providing some immediate relief to the patients. Table E.7-E.7a depict a plausible sample program that offers relief from a depressive disorder related to divorce.

### Commercial

### Advertising

### Example 8

World wide, companies spend more than $350 billion dollars a year advertising their products and services to consumers. Quantum Memory Fusion provides a fast and effective method for advertisers to reach viewers for substantially less. I estimate that a television advertiser could save 50-90% on advertising using my fusion methods. Companies continue to use the full context of their advertisements to indicate prices, products, and store locations. However, the fusion program becomes a permanent part of the advertisements, thereby reducing the number of showings or impressions required to get the message across to viewers. Yet another commercial possibility includes transmitting the fusion program into public spaces. For example, stores in a shopping mall could transmit short distance messages to passersby. Outdoor merchants could transmit their messages into lunchtime pedestrian traffic. Table E.8 illustrates examples of fusion programs that companies could annex into their radio, television, internet, or print media advertisements.

### Academic

### Time

### Example 9

Tables E.9, E.9a and E.9b illustrate a program to fuse the basic time-telling skills onto permanent recallable memory of a very young child (ages 2-5). Note that solution key dendrite 24 is an image that flashes for a duration equivalent to a word length in Table E.9. In Table E.9a, an abstract stream of the same data transmits simultaneously. The abstract query in Table E.9b queries the word “time” to make the neuronal association of time with the word “clock.” Because “clock” would be the index for time related fusion samples, a natural neuronal buffer 52 of substantial duration must separate each stream of time solution. The effect is a new solution neuron 92 for each time solution.

TABLE E.1
Academics (multiplication by 12) - Tactile and/or Abstract

| 12 | 14 | 16 | 18 | 20 | 22 | 24 | 26 |
|---|---|---|---|---|---|---|---|
| One | Times | Twelve | Suckers | The | Total | Twelve | Suckers |
| Two | Times | Twelve | Pears | This | Total | TwentyFour | Pears |
| Three | Times | Twelve | Candies | Only | Total | ThirtySix | Candies |
| Four | Times | Twelve | Oranges | Good | Total | FortyEight | Oranges |
| Five | Times | Twelve | Taffies | Great | Total | Sixty | Taffies |
| Six | Times | Twelve | Apples | Best | Total | SeventyTwo | Apples |
| Seven | Times | Twelve | Lemons | Winning | Total | EightyFour | Lemons |
| Eight | Times | Twelve | Cherries | Super | Total | NinetySix | Cherries |
| Nine | Times | Twelve | Grapes | Proper | Total | OneHundredEight | Grapes |
| Ten | Times | Twelve | Raisins | That | Total | OneHundredTwenty | Raisins |
| Eleven | Times | Twelve | Peaches | Correct | Total | OneHundredThirtyTwo | Peaches |
| Twelve | Times | Twelve | Cakes | Right | Total | OneHundredFortyFour | Cakes |

TABLE E.1a
Recitation Query (multiplication by 12) - Abstract

| 34 | 36 | 38 | 40 |
|---|---|---|---|
| Is | Twelve | Total | Suckers? |
| Is | TwentyFour | Total | Pears? |
| Is | ThirtySix | Total | Candies? |
| Is | FortyEight | Total | Oranges? |
| Is | Sixty | Total | Taffies? |
| Is | SeventyTwo | Total | Apples? |
| Is | EightyFour | Total | Lemons? |
| Is | NinetySix | Total | Cherries? |
| Is | OneHundredEight | Total | Grapes? |
| Is | OneHundredTwenty | Total | Raisins? |
| Is | OneHundredThirtyTwo | Total | Peaches? |
| Is | OneHundredFortyFour | Total | Cakes? |

TABLE E.2
Health Care (weight loss) - Tactile and/or Abstract

| 12 | 14 | 16 | 18 | 20 | 22 | 24 | 26 |
|---|---|---|---|---|---|---|---|
| First | Exercise | Eat | Salad | I | Shed | Weight | Salad |
| Second | Exercise | Eat | Fish | I | Shed | Fat | Fish |
| Third | Exercise | Eat | Fruit | I | Shed | Pounds | Fruit |
| Fourth | Exercise | Eat | Vegetables | I | Shed | Size | Vegetables |
| Fifth | Exercise | Eat | Soy | I | Shed | Inches | Soy |
| Sixth | Exercise | Grow | Confidence | I | Go | Jogging | Confidence |
| Seventh | Exercise | Build | Strength | I | Show | Stamina | Strength |
| Eighth | Exercise | Get | Happy | I | Am | Thin | Happy |
| Ninth | Exercise | New | Energy | I | Have | Fun | Energy |
| Tenth | Exercise | Be | Impressed | I | Love | Myself | Impressed |
| Eleventh | Exercise | No | Candy | I | Avoid | Junk | Candy |
| Twelfth | Exercise | No | Cake | I | Avoid | Fat | Cake |
| Thirteenth | Exercise | No | Pie | I | Avoid | Sugar | Pie |
| Fourteenth | Exercise | No | Donuts | I | Avoid | Starch | Donuts |
| Fifteenth | Exercise | No | Chips | I | Avoid | Sodium | Chips |
| Sixteenth | Exercise | No | Sweets | I | Avoid | Cavities | Sweets |
| Seventeenth | Exercise | No | Television | I | Avoid | Waste | Television |
| Eighteenth | Exercise | Do | 20 | I | Do | Sit-ups | 20 |
| Nineteenth | Exercise | Do | 30 | I | Do | Pushups | 30 |
| Twentieth | Exercise | Do | 40 | I | Do | JumpingJacks | 40 |
| Twenty | First | Do | 50 | I | Do | Leglifts | 50 |
| Twenty | second | Do | Daily | I | Do | Exercise | Daily |

TABLE E.3
Health Care (tobacco aversion) - Tactile and/or Abstract

| 12 | 14 | 16 | 18 | 20 | 22 | 24 | 26 |
|---|---|---|---|---|---|---|---|
| First | Stop | Using | Ciga- | I | Quit | Smoking | Cigarettes |
| | | | rettes | | | | |
| Second | Stop | Drinking | Coffee | I | Quit | Caffeine | Coffee |
| Third | Save | Your | Life | I | Quit | Dying | Life |
| Fourth | Save | Your | Lungs | I | Quit | Tobacco | Lungs |
| Fifth | Save | Your | Money | I | Quit | Spending | Money |

TABLE E.3a
Action Query (tobacco aversion) - Abstract

| 34 | 36 | 38 | 40 |
|---|---|---|---|
| When | Thinking | Of | Cigarettes? |

TABLE E.4
Self Help (leadership traits) - Tactile and Abstract

| 12 | 14 | 16 | 18 | 20 | 22 | 24 | 26 |
|---|---|---|---|---|---|---|---|
| First | Prepare | Grow | Reward | I | Practice | Justice | Reward |
| Second | Learn | Discern | Facts | I | Practice | Judgement | Facts |
| Third | Strength | Firm | Ability | I | Practice | Decisiveness | Ability |
| Fourth | Candor | Truth | Honesty | I | Practice | Integrity | Honesty |
| Fifth | Never | Quit | Performance | I | Practice | Dependability | Performance |
| Sixth | Be | Humble | Respect | I | Practice | Tact | Respect |
| Seventh | Always | First | Action | I | Practice | Initiative | Action |
| Eighth | Extra | Effort | Stamina | I | Practice | Endurance | Stamina |
| Ninth | Stand | Tall | Conduct | I | Practice | Bearing | Conduct |
| Tenth | Give | More | Generous | I | Practice | Unselfishness | Generous |
| Eleventh | No | Fear | Bravery | I | Practice | Courage | Bravery |
| Twelfth | Be | Prudent | Wisdom | I | Practice | Knowledge | Wisdom |
| Thirteenth | Semper | Fidelis | Faithful | I | Practice | Loyalty | Faithful |
| Fourteenth | Passion | Fervor | Zeal | I | Practice | Enthusiasm | Zeal |

TABLE E.4a
Action Query (leadership traits) - Abstract

| 34 | 36 | 38 | 40 |
|---|---|---|---|
| What | Do | I | Practice? |

TABLE E.5
Academics (English vocabulary) - Tactile and Abstract

| 12 | 14 | 16 | 18 | 20 | 22 | 24 | 26 |
|---|---|---|---|---|---|---|---|
| First | Word | Exalt | Ordain | Exalt | Means | Sanctify | Ordain |
| Second | Word | Urgent | Sharp | Urgent | Means | Acute | Sharp |
| Third | Word | Expert | Skilled | Expert | Means | Adept | Skilled |
| Fourth | Word | Combine | Add | Combine | Means | Adjoin | Add |
| Fifth | Word | Adept | Smart | Adept | Means | Adroit | Smart |
| Sixth | Word | Excited | Eager | Excited | Means | Agog | Eager |
| Seventh | Word | Recess | Nook | Recess | Means | Alcove | Nook |
| Eighth | Word | Satiate | Relieve | Satiate | Means | Allay | Relieve |
| Ninth | Word | Region | Circuit | Region | Means | Ambit | Circuit |
| Tenth | Word | Settle | Restrain | Settle | Means | Anchor | Restrain |
| Eleventh | Word | Abrade | Injure | Abrade | Means | Chaff | Injure |
| Twelfth | Word | Dissenter | Defector | Dissenter | Means | Apostate | Defector |
| Thirteenth | Word | Paltry | Evil | Paltry | Means | Arrant | Evil |
| Fourteenth | Word | Incline | Fly | Incline | Means | Ascend | Fly |
| Fifteenth | Word | Cheat | Amuse | Cheat | Means | Beguile | Amuse |
| Sixteenth | Word | Refuge | Home | Refuge | Means | Asylum | Home |
| Seventeenth | Word | Humble | Abject | Humble | Means | Servile | Abject |
| Eighteenth | Word | Strict | Plain | Strict | Means | Austere | Plain |
| Nineteenth | Word | Testify | Confess | Testify | Means | Avow | Confess |
| Twentieth | Word | Concise | Abridged | Concise | Means | Condensed | Abridged |

TABLE E.5a
Recitation Query (English vocabulary) - Abstract

| 34 | 36 | 38 | 40 |
|---|---|---|---|
| What | Does | Ordain | Mean? |
| What | Does | Sharp | Mean? |
| What | Does | Skilled | Mean? |
| What | Does | Add | Mean? |
| What | Does | Smart | Mean? |
| What | Does | Eager | Mean? |
| What | Does | Nook | Mean? |
| What | Does | Relieve | Mean? |
| What | Does | Circuit | Mean? |
| What | Does | Restrain | Mean? |
| What | Does | Injure | Mean? |
| What | Does | Defector | Mean? |
| What | Does | Evil | Mean? |
| What | Does | Fly | Mean? |
| What | Does | Amuse | Mean? |
| What | Does | Home | Mean? |
| What | Does | Abject | Mean? |
| What | Does | Plain | Mean? |
| What | Does | Confess | Mean? |
| What | Does | Abridged | Mean? |

TABLE E.6
Academics (Spanish vocabulary) - Tactile and Abstract

| 12 | 14 | 16 | 18 | 20 | 22 | 24 | 26 |
|---|---|---|---|---|---|---|---|
| First | Word | Exalt | Ordain | Exalt | Spanish | Sanctifar | Ordain |
| Second | Word | Urgent | Sharp | Urgent | Spanish | Agudo | Sharp |
| Third | Word | Expert | Skilled | Expert | Spanish | Experto | Skilled |
| Fourth | Word | Combine | Add | Combine | Spanish | Colindar | Add |
| Fifth | Word | Adept | Smart | Adept | Spanish | Diestro | Smart |
| Sixth | Word | Excited | Eager | Excited | Spanish | Ardiente | Eager |
| Seventh | Word | Recess | Nook | Recess | Spanish | Gabinete | Nook |
| Eighth | Word | Satiate | Relieve | Satiate | Spanish | Alliviar | Relieve |
| Ninth | Word | Region | Circuit | Region | Spanish | Región | Circuit |
| Tenth | Word | Settle | Restrain | Settle | Spanish | Ancla | Restrain |
| Eleventh | Word | Abrade | Injure | Abrade | Spanish | Barcia | Injure |
| Twelfth | Word | Dissenter | Defector | Dissenter | Spanish | Apóstol | Defector |
| Thirteenth | Word | Paltry | Evil | Paltry | Spanish | Malvado | Evil |
| Fourteenth | Word | Incline | Fly | Incline | Spanish | Subir | Fly |
| Fifteenth | Word | Cheat | Amuse | Cheat | Spanish | Engañar | Amuse |
| Sixteenth | Word | Refuge | Home | Refuge | Spanish | Asilo | Home |
| Seventeenth | Word | Humble | Abject | Humble | Spanish | Servil | Abject |
| Eighteenth | Word | Strict | Plain | Strict | Spanish | Austero | Plain |
| Nineteenth | Word | Testify | Confess | Testify | Spanish | Admitir | Confess |
| Twentieth | Word | Concise | Abridged | Concise | Spanish | Condensar | Abridged |

TABLE E.6a
Recitation Query (Spanish vocabulary) - Abstract

| 34 | 36 | 38 | 40 |
|---|---|---|---|
| Ordain | In | Spanish | Means? |
| Sharp | In | Spanish | Means? |
| Skilled | In | Spanish | Means? |
| Add | In | Spanish | Means? |
| Smart | In | Spanish | Means? |
| Eager | In | Spanish | Means? |
| Nook | In | Spanish | Means? |
| Relieve | In | Spanish | Means? |
| Circuit | In | Spanish | Means? |
| Restrain | In | Spanish | Means? |
| Injure | In | Spanish | Means? |
| Defector | In | Spanish | Means? |
| Evil | In | Spanish | Means? |
| Fly | In | Spanish | Means? |
| Amuse | In | Spanish | Means? |
| Home | In | Spanish | Means? |
| Abject | In | Spanish | Means? |
| Plain | In | Spanish | Means? |
| Confess | In | Spanish | Means? |
| Abridged | In | Spanish | Means? |

TABLE E.7
Mental Health (depression related to divorce) - Tactile and Abstract

| 12 | 14 | 16 | 18 | 20 | 22 | 24 | 26 |
|---|---|---|---|---|---|---|---|
| First | I | Am | Free | I | Feel | Better | Free |
| Second | I | Am | Good | I | Feel | Worthy | Good |
| Third | I | Am | Intelligent | I | Feel | Capable | Intelligent |
| Fourth | I | Am | Loving | I | Feel | Connected | Loving |
| Fifth | I | Am | Generous | I | Feel | Forgiven | Generous |
| Sixth | I | Am | Reliable | I | Feel | Stable | Reliable |
| Seventh | I | Am | Robust | I | Feel | Powerful | Robust |
| Eighth | I | Am | Secure | I | Feel | Fearless | Secure |
| Ninth | I | Am | Clear | I | Feel | Positive | Clear |
| Tenth | I | Am | Rested | I | Feel | Relieved | Rested |

TABLE E.7a
Recitation Query (depression related to divorce) - Abstract Only

| 34 | 36 | 38 | 40 |
|---|---|---|---|
| How | Do | I | Feel |

TABLE E.8
Commercial (advertising) - Tactile and Abstract

| 12 | 14 | 16 | 18 | 20 | 22 | 24 | 26 |
|---|---|---|---|---|---|---|---|
| Zero | Down | At | Joe's | Visit | Our | Showroom | Joe's |
| Zero | Interest | For | 90 | Days | That's | Right | 90 |
| Three | Day | Special | Event | Save | Today | Super | Event |
| One | Call | Puts | You | In | The | Car-that-wants | You |
| One | Day | Only | Sale | Everything | Must | Go | Sale |
| Four | The | Best | Prices | Shop | Our | Everyday | Prices |
| Forty | Eight | Hour | Sale | Our | Last | Half-off | Sale |
| First | 50 | Customers | Win | Come | Today | And | Win |
| Fifty | Million | Bankruptcy | Liquidation | Court | Ordered | Inventory | Liquidation |

TABLE E.9.
Academic (time) - Tactile and Abstract

| 12 | 14 | 16 | 18 | 20 | 22 | 24 | 26 |
|---|---|---|---|---|---|---|---|
| One | Three | O | Clock | Three | O | 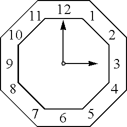 | Clock |
| One | Three | O | Clock | Three | O | Time | Clock |

TABLE E.9a.
Academic (time) - Tactile and Abstract

| 12 | 14 | 16 | 18 | 20 | 22 | 24 | 26 |
|---|---|---|---|---|---|---|---|
| One | 3 | O | Clock | Three | O | 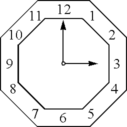 | Clock |
| One | Three | O | Clock | Three | O | Time | Clock |

TABLE E.9b
Recitation Query (time) - Abstract Only

| 34 | 36 | 38 | 40 |
|---|---|---|---|
| The | Clock | Time | Is? |

### SUMMARY, RAMIFICATION, AND SCOPE

The reader will thus see that I have invented a particularly simple method for fusing a plurality of data types onto permanent recallable memory of humans and other animals. Quantum Memory Fusion is new, innovative, and unobvious, and there are no other chemical free methods known in the prior art, in industry or in the world that communicate at the molecular level with neurons in a brain. Quantum Memory Fusion is an all natural universal human technology. The method uses formulas and data arrangements that facilitate fusion of data onto brain cells called neurons.

Consumers spend $780 billion dollars annually on postsecondary education according to a Business week article of Jan. 10, 2000. The methods of Quantum Memory Fusion are less costly than a single hour of classroom instruction in a major university, yet the method is capable of fusing onto permanent recallable memory many times more data in the same one hour than can be learned traditionally in one hour.

Corporate for-profit education, according to a Business week article from January 2000, generates an estimated $63 billion dollars a year. The invention described herein will save corporations billions of dollars by reducing the time required to attend corporate programs and by delivering the content to the memory of the employee quickly, inexpensively, and painlessly while the employee remains onsite.

Children tend to have anxieties about mathematics. Quantum Memory Fusion overcomes these anxieties by fusing the equations and solutions to molecular memory. The children's educational toy industry is worth $4 billion dollars annually according to a Harvard Business school report of Apr. 5, 2004. Educational toys built on Quantum Memory Fusion methods will have greater value to the consumer, impressive results for the children, and will bring higher prices for the manufacturers. Fuse the foundation of mathematics, reading, and social behavior onto the permanent recallable memory of a child during the formative years, and that child will have a greater desire to learn and explore. Additionally, a well-rounded young person encounters greater employment and social opportunities in adulthood.

Quantum Memory Fusion advances the speed of learning. The possibility that sixth and seventh grade students will study from college equivalent text is very real in the not so distant future of public education.

The San Mateo county Times, of San Mateo Calif. published an article on Apr. 6, 2003, estimating that the electronic video game industry is worth $10.3 billion dollars annually. Electronic video games can teach the rules, methods, and secrets of the games quickly with Quantum Memory Fusion and serve as allies to parents who want to teach morality and ethics to their children.

According to a Jan. 22, 2002 article in Forbes, self improvement product sales rose to $4.7 billion annually, with an anticipated growth rate of 12% per year. The key to self-improvement is memory. Remembering the principles of success, social behavior, weight management, or good health habits is the only way to bring about these improvements. The methods of the invention produce neuromuscular responses to external stimulus as well as behavior modification with little to no effort from the user. The invention uses innovative Action Queries to produce physical activity based on normal occurrences in the general human environment. The teachings of celebrated personal development trainers such as Anthony Robbins, the late Earl Nightingale, Jay Abraham, Dennis Waitley, and Stuart Wilde transmit and fuse effortlessly to the permanent recallable memory of listeners, thereby producing immediate results for a fraction of the current cost.

Companies will save billions of dollars on advertising expenses with the present invention and make more profit per product sold. Quantum Memory Fusion fuses any data to permanent recallable memory including advertising messages. Regardless of the method of advertisement, viewers hear, see, read, and remember the messages. A Microsoft Encarta online Encyclopedia 2004 report entitled “Advertising” stated that advertising spending worldwide exceeds $350 billion dollars.

Medicaid spent over $21 billion dollars for the treatment of mental healthcare and addictive disorders in 1997 according to a February 2003 article in Health Affairs from the American Psychiatric Association. One third of Americans suffer from some form of mental or addictive disorder, yet most mentally ill Americans receive no active treatment according to Dr. Richard C. W. Hall, medical director of psychiatric programs Florida Hospital. Dr. Hall wrote that the U.S. spent $273.3 billion dollars servicing mental and addictive disorders in 1985. Quantum Memory Fusion methods can service behavior modification because any external agent could, by coinciding in time with an ordinary reflex, become the conditioned signal for the formation of a new conditioned reflex. In other words, the present invention can create a new reflex to existing behaviors by fusing the desired result of a new behavior onto molecular memory, and then fusing an abstract query against the behavioral model onto the same neuronal memory block. The abstract query is self-perpetuating in that it will pose its query autonomously. This groundbreaking method termed Reflexive Memory Recall is unsurpassed in all of psychology.

When partnered with virtual therapy, the present invention creates a new brand of universal human technology. For example, virtual therapy is becoming a popular method for treating numerous phobias and mental illnesses. Even virtual therapy alone can not produce the fast, permanent, and inexpensive results of the present invention. Virtual therapy devices built on Quantum Memory Fusion methods will usher in an entirely new age of medical diagnostics and treatment of psychological illnesses, chronic dependencies and behavioral disorders. I estimate that patients would prefer to visit inexpensive virtual therapy kiosk equipped with Quantum Memory Fusion over visiting therapists for treatment of simple phobias and other mental disorders that would not normally require pharmaceuticals or extensive office visits.

We no longer live in a society of close-knit townships and tribal elders. No one is there to teach morals and ethics to our children in our absence. We are responsible for the kinds of people we produce. When the moral fiber of a nation is in question, it is the responsibility of the current adult generation to replenish the moral and ethical sustenance for subsequent generations. Parents of this generation bear a responsibility greater than their own parents bore. They have less family and leisure time and usually both parents work outside the home. The nation's children learn morals and ethics from television shows, magazines or from their peers. Our children are overweight and under nourished both physically and psychologically. None of these maladies is suitable. Quantum Memory Fusion allows all parents to instill their values into their children, even while their children sleep. We often talk to our children while they sleep, hoping they can hear us—knowing that they can not. Sleep Trainers are the unobtrusive programs that fuse data (in this case moral behavior) deep inside the memory of the child for the duration of that child's sleep cycle. The advantages are obvious, there are no rebellious outburst or peer-pressured objections from the child, and the message is “heard”—loud and clear.

Often people wish that they could be like someone else. Usually, the admired is a famous person, while sometimes it is a coworker or a friend. This harmless admiration could never become reality for the admirers until the present invention. Quantum Memory Fusion methods include the Incognizant Personality construct that literally fuses the traits of the admired onto the permanent recallable memory of the admirer.

I foresee the average person having the future opportunity to purchase inexpensively the personality constructs of any one of or all of the most famous people that have ever lived, to include doctors, lawyers, engineers, inventors, athletes, financiers, Nobel winners, chemists, scientists, writers, poets, musicians, actors, or simply personality constructs that promote harmonious and healthy living.

More products, books, and services will depend on Quantum Memory Fusion methods than any other methods in the history of humankind.

Quantum Memory Fusion methods will be the primary reasons for compounding scientific and human advances in all areas of life. Kelly's Query Funnel is yet another powerful tool incorporated by the invention. Man's thirst for knowledge, solutions, and new ideas is more feasible at this very moment than at any other time in all of the preceding 10,000 years of civilization.

The brain is the most complex device in the known universe. At only 3 pounds, it has the unimaginable power to bring about all possible things. Quantum Memory Fusion reveals its internal workings to an extent never before imagined. The present invention exposes the methods, procedures, plans, goals, and survival techniques of the brain. Quantum Memory Fusion gives to the brain its payload on a silver platter and the brain responds with powerful new memories. Quantum Memory Fusion is the only artificial method that has ever created a symbiotic relationship with the brain without intra-cranial intrusion, drugs, or electronic devices. Quantum Memory Fusion answers the three hundred-year-old question.

In accessing the infinite storage capacity of the brain, one of my discoveries focused on the slow pace at which we learn. I found that because the human brain evolved before spoken language, our brains were denied the proper data structures at the time man began to communicate. Consequently, humankind forces the brain to work backwards with fragmented data (data similar in structure to man's first sounds). My discoveries prove that evolution is the key. I theorize that the human brain is unformatted in that its data structuring and organization methods remain primitive—hence our inability to create uniform genius or use more of our massive storage capacity during a normal life time. I further theorize that the application of my technology during the embryonic stage would in fact harmlessly format the developing brain, resulting in a near perfect and immediate assimilation of information at birth that would continue throughout life. This “early formatting” would clearly pave the way to 7-year old high school graduates and 12-year old college graduates as the intrinsic learning cycle versus the anomalous prodigy.

The numerous accomplishments within my laboratory foster high expectations for long-term success in industry and global distribution and acceptance within weeks of public announcement.

In summary and I quote, “there can be no sensory stimulation that bypasses memorization regardless of the method of data acquisition, be it concrete or construct, conscious or subconscious, real or fantasy, tangible or intangible, physical or phantom, the core of all perception is memory, else all stimuli are meaningless objects and occurrences of perpetual first presentation”—Dr. Frederick Mitchell.

Although the description above contains many specificities, these should not be construed as limiting the scope of the invention but as merely providing illustrations of some of the presently preferred embodiments of this invention. For example, Quantum Memory Fusion can be the basis of neuro-software programs for the brain much like DOS (disc operating system) is the basis of the Microsoft® Windows® operating system and third party software add-ons. Another example could include Quantum Memory Fusion as the transport mechanism in neuro sequencing and external neuro transmissions.

Thus, the scope of the invention should be determined by the appended claims and their legal equivalents, rather than by the examples given.

I claim:

1. A method for storing, retrieving, appending, erasing, updating, manipulating, querying, creating sensory data, comprising the steps of:
(a) a first impulse signal energy,
(b) a second impulse signal energy,
(c) a third impulse signal energy,
(d) a fourth impulse signal energy,
(e) a fifth impulse signal energy,
(f) a sixth impulse signal energy,
(g) a seventh impulse signal energy,
(h) an eighth impulse signal energy, and
(i) with said impulse signal energy based on input sensory data combined toward one or more or a combination of sensory receptors selected from the group consisting of human, animal, plant, mechanical, or electronic sensory receptors of similar purpose and means.

2. The method of claim 1, further comprising:
(a) an impulse signal energy of adequate duration to buffer;
(1) the first impulse signal energy from the second impulse signal energy,
(2) the second impulse signal energy from the third impulse signal energy,
(3) the third impulse signal energy from the fourth impulse signal energy,
(4) the fourth impulse signal energy from the fifth impulse signal energy,
(5) the fifth impulse signal energy from the sixth impulse signal energy,
(6) the sixth impulse signal energy from the seventh impulse signal energy, and
(7) the seventh impulse signal energy from the eight impulse signal energy,

3. The method of claim 1, further comprising:
(a) input data consisting of a query containing a plurality of impulse signal energy structures combined toward one or more or a combination of sensory receptors selected from the group consisting of human, animal, plant, mechanical, or electronic sensory receptors of similar purpose and means.
